(19) United States
(12) Patent Application Publication
(10) Pub. No.: US 2024/0076708 A1
(43) Pub. Date: Mar. 7, 2024

(54) HIGH-PURITY STEVIOL GLYCOSIDES

(71) Applicant: PURECIRCLE SDN BHD, KUALA LUMPUR (MY)

(72) Inventors: Avetik MARKOSYAN, Kuala Lumpur (MY); Indra PRAKASH, Alpharetta, GA (US); Cynthia BUNDERS, Atlanta, GA (US); Pankaj SONI, Kennesaw, GA (US); Cyrille JARRIN, Muret (FR); Aurélien BADIE, Labège (FR); Robert TER HALLE, Baziege (FR)

(21) Appl. No.: 18/061,861

(22) Filed: Dec. 5, 2022

Related U.S. Application Data
(63) Continuation of application No. 16/903,369, filed on Jun. 16, 2020, now Pat. No. 11,542,537.
Application No. 16/903,369 is a continuation of application No. 15/694,524, filed on Sep. 1, 2017, now Pat. No. 10,683,526.
Application No. 15/694,524 is a continuation of application No. 14/287,837, filed on May 27, 2014, now Pat. No. 9,752,174.
Application No. 15/694,524 is a continuation-in-part of application No. 15/400,325, filed on Jan. 6, 2017, now Pat. No. 10,485,257.
Application No. 15/400,325 is a continuation of application No. 14/954,213, filed on Nov. 30, 2015, now abandoned.
Application No. 14/954,213 is a division of application No. 14/469,076, filed on Aug. 26, 2014, now Pat. No. 9,243,273.
Application No. 14/469,076 is a continuation of application No. PCT/US2013/030439, filed on Mar. 12, 2013.
(60) Provisional application No. 61/861,528, filed on Aug. 2, 2013.
(60) Provisional application No. 61/881,166, filed on Sep. 23, 2013.
(60) Provisional application No. 61/885,084, filed on Oct. 1, 2013.
(60) Provisional application No. 61/904,751, filed on Nov. 15, 2013.
(60) Provisional application No. 61/913,482, filed on Dec. 9, 2013.
(60) Provisional application No. 61/921,635, filed on Dec. 30, 2013.
(60) Provisional application No. 61/925,329, filed on Jan. 9, 2014.
(60) Provisional application No. 61/827,922, filed on May 28, 2013.
(60) Provisional application No. 61/939,855, filed on Feb. 14, 2014.
(60) Provisional application No. 61/649,978, filed on May 22, 2012.

(51) Int. Cl. C12P 19/56; A23L 2/60; A23L 27/30; A24B 15/10; A24B 15/30; C07H 15/256

(52) U.S. Cl. CPC C12P 19/56; A23L 2/60; A23L 27/33; A24B 15/10; A24B 15/302; C07H 15/256; A23V 2002/00; Y02P 20/582

(57) ABSTRACT

Methods of preparing highly purified steviol glycosides, particularly rebaudiosides A, D and M are described. The methods include utilizing recombinant microorganisms for converting various staring compositions to target steviol glycosides. In addition, novel steviol glycosides reb D2 and reb M2 are disclosed, as are methods of preparing the same. The highly purified rebaudiosides are useful as non-caloric sweetener in edible and chewable compositions such as any beverages, confectioneries, bakery products, cookies, and chewing gums.

### RELATED APPLICATIONS

The present application is a continuation application of U.S. patent application Ser. No. 16/903,369, filed on Jun. 16, 2020, which is a continuation application of U.S. patent application Ser. No. 15/694,524, filed on Sep. 1, 2017, which issued as U.S. Pat. No. 10,683,526, which is a continuation application of U.S. patent application Ser. No. 14/287,837, filed on May 27, 2014, which issued as U.S. Pat. No. 9,752,174 on Sep. 5, 2017, and which claims the benefit of priority from U.S. Provisional Application No. 61/827,922, filed on May 28, 2013, U.S. Provisional Application No. 61/843,544, filed on Jul. 8, 2013, U.S. Provisional Application No. 61/861,528, filed on Aug. 2, 2013, U.S. Provisional Application No. 61/881,166, filed on Sep. 23, 2013, U.S. Provisional Application No. 61/885,084, filed on Oct. 1, 2013, U.S. Provisional Application No. 61/904,751, filed on Nov. 15, 2013, U.S. Provisional Application No. 61/913,482, filed on Dec. 9, 2013, U.S. Provisional Application No. 61/921,635, filed on Dec. 30, 2013, U.S. Provisional Application No. 61/925,329, filed on Jan. 9, 2014, and U.S. Provisional Application No. 61/939,855, filed on Feb. 14, 2014. The present application is also a continuation-in-part application of U.S. patent application Ser. No. 15/400,325, filed on Jan. 6, 2017, which is a continuation application of U.S. patent application Ser. No. 14/954,213, filed on Nov. 30, 2015, now abandoned, which is a divisional application of U.S. patent application Ser. No. 14/469,076, filed on Aug. 26, 2014, which issued as U.S. Pat. No. 9,243,273 on Jan. 26, 2016, and which is a continuation application of International Application No. PCT/US2013/030439, filed on Mar. 12, 2013, which claims the benefit of priority from U.S. Provisional Application No. 61/649,978, filed on May 22, 2012. The contents of which applications are incorporated herein by reference in their entireties.

### REFERENCE TO AN ELECTRONIC SEQUENCE LISTING

The contents of the electronic sequence listing entitled “6070_SWT_US_CIP_CON3_ST_26_sequence.xml,” created on Dec. 5, 2022, having 21 kilobytes of data, and filed concurrently herewith, is herein incorporated by reference in its entirety.

### TECHNICAL FIELD

The present invention relates to a biocatalytic process for preparing compositions comprising steviol glycosides, including highly purified steviol glycoside compositions. The present invention also relates to novel steviol glycosides, methods for isolation of the same and uses for the novel steviol glycosides.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcosideA (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

Additionally, there remains a need for novel steviol glycosides and methods of preparing and isolating the same.

### SUMMARY OF THE INVENTION

The present invention provides a biocatalytic process for preparing a composition comprising a target steviol glycoside by contacting a starting composition comprising an organic substrate with a microorganism and/or biocatalyst, thereby producing a composition comprising a target steviol glycoside.

The starting composition can be any organic compound comprising at least one carbon atom. In one embodiment, the starting composition is selected from the group consisting of polyols or sugar alcohols, various carbohydrates.

The target steviol glycoside can be any steviol glycoside. In one embodiment, the target steviol glycoside is steviolmonoside, steviolbioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside M2, rebaudioside D, rebaudioside D2, rebaudioside N, rebaudioside O or a synthetic steviol glycoside.

In one embodiment, the target steviol glycoside is stevioside.

In another embodiment, the target steviol glycoside is rebaudioside A.

In still another embodiment, the target steviol glycoside is rebaudioside D.

In yet another embodiment, the target steviol glycoside is rebaudioside M (also known as rebaudioside X).

The microorganism can be any microorganism possessing the necessary enzymes for converting the starting composition to target steviol glycosides.

The biocatalysts will comprise at least one enzyme for converting the starting composition to target steviol glycosides.

The biocatalysts can be located on the surface and/or inside the cell of the microorganism or can be secreted out of the microorganism.

The biocatalyst can be whole cell suspension, crude lysate or purified enzymes.

The biocatalyst can be in free form or immobilized to a solid support made from inorganic or organic materials.

The enzymes necessary for converting the starting composition to target steviol glycosides include the steviol biosynthesis enzymes, UDP-glycosyltransferases (UGTs) and/or UDP-recycling enzyme.

In one embodiment the steviol biosynthesis enzymes include mevalonate (MVA) pathway enzymes.

In another embodiment the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to the steviol and or steviol glycoside substrate to provide the target steviol glycoside.

In one embodiment, steviol biosynthesis enzymes and UDP-glucosyltransferases are produced in a microorganism. The microorganism may be, for example, E. coli, Saccharomyces sp., Aspergillus sp., Pichia sp,. Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2 and UGTs having substantial (>85%) identity to these polypeptides as well as isolated nucleic acid molecules that code for these UGTs.

In one embodiment, steviol biosynthesis enzymes, UGTs and UDP-glucose recycling system are present in one microorganism. The microorganism may be for example, E. coli, Saccharomyces sp., Aspergillus sp., Pichia sp,. Bacillus sp., Yarrowia sp.

In one embodiment, the UDP-glucosyltransferase is any UDP-glucosyltransferase capable of adding at least one glucose unit to rubusoside to form stevioside. In a particular embodiment, the UDP-glucosyltransferase is UGT91D2.

In one embodiment, the UDP-glucosyltransferase is any UDP-glucosyltransferase capable of adding at least one glucose unit to stevioside to form rebaudioside A. In a particular embodiment, the UDP-glucosyltransferase is UGT76G1.

In another embodiment, the UDP-glucosyltransferase is any UDP-glucosyltransferase capable of adding at least one glucose unit to rebaudioside A to form rebaudioside D. In a particular embodiment, the UDP-glucosyltransferase is UGT91D2. In another embodiment, the UGT is an improved variant of UGT91D2 with higher activity and/or selectivity produced by directed evolution.

In yet another embodiment, the UDP-glucosyltransferase is any UDP-glucosyltransferase capable of adding at least one glucose unit to rebaudioside D to form rebaudioside M. In a particular embodiment, the UDP-glucosyltransferase is UGT76G1. In another embodiment, the UGT is an improved variant of UGT76G1 with higher activity and/or selectivity produced by directed evolution.

Optionally, the method of the present invention further comprises recycling UDP to provide UDP-glucose. In one embodiment, the method comprises recycling UDP by providing a recycling catalyst and a recycling substrate, such that the biotransformation of the steviol glycoside substrate to the target steviol glycoside is carried out using catalytic amounts of UDP-glucosyltransferase and UDP-glucose (FIG. 3).

In one embodiment, the recycling catalyst is sucrose synthase.

In one embodiment, the recycling substrate is sucrose.

Optionally, the method of the present invention further comprises separating the target steviol glycoside from the starting composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

The target steviol glycoside can be in any polymorphic or amorphous form, including hydrates, solvates, anhydrous or combinations thereof.

Purified target steviol glycosides can be used in consumable products as a sweetener. Suitable consumer products include, but are not limited to, food, beverages, pharmaceutical compositions, tobacco products, nutraceutical compositions, oral hygiene compositions, and cosmetic compositions.

The present invention also provides novel steviol glycosides rebaudioside D2 (reb D2, isomer of rebaudioside D) and rebaudioside M2 (reb M2, isomer of rebaudioside M), which are isomers of reb D and reb M, respectively. In one embodiment, isolated and purified reb D2 is provided. In another embodiment, isolated and purified reb M2 is provided. Reb D2 and reb M2 may also be present in any consumable products disclosed herein. In a particular embodiment, beverages comprising reb D2 and/or reb M2 are provided.

Methods of preparing reb D2 and reb M2 are also provided herein. Both are formed during the biotransformation of reb A to reb D. Reb M2 is believed to form from biotransformation of reb D2 in situ.

In one embodiment, the present invention is a method for the preparation of a composition comprising reb D2 comprising: (a) contacting a starting composition comprising reb A with an enzyme capable of transforming reb A to reb D2, UDP-glucose, and optionally UDP-glucose recycling enzymes, to produce a composition comprising reb D2, and (b) isolating the composition comprising reb D2.

In another embodiment, the present invention is a method for the preparation of a composition comprising reb M2 comprising (a) contacting a starting composition comprising reb D2 with an enzyme capable of transforming reb D2 to reb M2, UDP-glucose, and optionally UDP-glucose recycling enzymes, to produce a composition comprising reb M2, and (b) and isolating the composition comprising reb M2.

A further method for the preparation of a composition comprising reb M2 comprises (a) contacting a starting composition comprising reb A with an enzyme capable of transforming reb A to reb D2, UDP-glucose, and optionally UDP-glucose recycling enzymes, to produce a composition comprising reb D2, (b) optionally, isolating the composition comprising reb D2, (c) contacting the composition comprising reb D2 with an enzyme capable of transforming reb D2 to reb M2, UDP-glucose, and optionally UDP-glucose recycling enzymes to produce a composition comprising reb M2, and (d) isolating the composition comprising reb M2.

The composition can be further purified to provide reb D2 or reb M2 with purities greater than about 95% by weight on a dry basis.

### BRIEF DESCRIPTION OF THE DRAWINGS

The accompanying drawings are included to provide a further understanding of the invention. The drawings illustrate embodiments of the invention and together with the description serve to explain the principles of the embodiments of the invention.

FIG. 1 shows the structure of reb M.

FIG. 2 shows the biocatalytic production of reb M from stevioside.

FIG. 3 shows the biocatalytic production of reb A from stevioside using the enzyme UGT76G1 and concomitant recycling of UDP to UDP glucose via sucrose synthase.

FIG. 4 shows the IR spectrum of reb M.

FIG. 5. shows the HPLC chromatogram of the product of the biocatalytic production of reb M from reb D, as detailed in Example 14. The peak with retention time of 24.165 minutes corresponds to unreacted reb D. The peak with retention time of 31.325 minutes corresponds to reb M.

FIG. 6. shows the HPLC chromatogram of purified reb M produced by biocatalysis from reb D.

FIG. 7 shows the HPLC chromatogram of a reb M standard.

FIG. 8 shows the HPLC chromatogram of co-injection of a reb M standard and reb M purified from biotransformation from reb D.

FIG. 9 shows an overlay of the ¹H NMR spectra of a reb M standard and reb M purified following biosynthesis from reb D.

FIG. 10 shows the HRMS spectrum of reb M purified following biocatalytic production from reb D.

FIG. 11 shows LC-MS analysis of semi-synthetic steviol glycoside mixture, Lot number CB-2977-106, showing TIC (A), MS of peak at 1.8 min (B), MS of reb M2 peak at 4.1 min (C), MS of reb D peak at 6.0 min (D), MS of reb D2 peak at 7.7 min (E), MS of peak at 9.4 min (F), MS of rebaudioside Apeak at 15.2 min (G), MS of peak at 16.5 min (H), and MS of peak at 18.3 min (I).

FIG. 12 shows the trace of semi-synthetic steviol glycoside mixture, Lot number CB-2977-106. Chromatogram gridlines are not homogeneous as the detector was re-calibrated 14 min following injection.

FIG. 13 shows HPLC analysis of semi-synthetic steviol glycoside mixture, Lot number CB-2977-106 (A), Isolated reb M2 (B), isolated reb D (C) and isolated reb D2 (D).

FIG. 14 shows the ¹H NMR spectrum of reb D2 (500 MHz, pyridine-d₅).

FIGS. 15A and 15B show the ¹³C NMR spectrum of reb D2(125 MHz, pyridine-d₅).

FIGS. 16A and 16B show an expansion of the ¹³C NMR spectrum of reb D2 (125 MHz, pyridine-d₅).

FIGS. 17A and 17B show the ¹H-¹H COSY Spectrum of reb D2 (500 MHz, pyridine-d₅).

FIGS. 18A and 18B show the HSQC-DEPT spectrum of reb D2(500 MHz, pyridine-d₅).

FIG. 19 shows the HMBC spectrum of reb D2.

FIG. 20 shows an expansion of HMBC spectrum of reb D2 (500 MHz, pyridine-d₅).

FIG. 21 shows the ¹H NMR spectrum of reb M2(500 MHz, D20).

FIG. 22 shows the ¹³C NMR spectrum of reb M2 (125 MHz, D20/TSP).

FIG. 23 shows an expansion of the ¹³C NMR spectrum of reb M2 (125 MHz, D20/TSP).

FIG. 24 shows the ¹H-¹H COSY spectrum of reb M2 (500 MHz, D20).

FIG. 25 shows the HSQC-DEPT spectrum of reb M2(500 MHz, D20).

FIG. 26 shows the HMBC spectrum of reb M2 (500 MHz, D20).

FIGS. 27A and 27B show an expansion of HMBC spectrum of reb M2 (500 MHz, D20).

FIGS. 28A and 28B show another HMBC spectrum of reb M2.

FIGS. 29A and 29B show a ¹H NMR spectrum of reb M2.

FIGS. 30A and 30B show a ¹³C NMR spectrum of reb M2

FIGS. 31A and 31B show another ¹³C NMR spectrum of reb M2.

FIGS. 32A and 32B show a ¹H-¹H COSY spectrum of reb M2.

FIGS. 33A and 33B show a HSQC-DEPT spectrum of reb M2.

FIGS. 34A and 34B show an HMBC spectrum of reb M2.

FIGS. 35A and 35B show another HMBC spectrum of reb M2.

FIG. 36 shows a 1D-TOCSY spectrum of reb M2.

FIG. 37 shows a 1D-TOCSY spectrum of reb M2.

FIG. 38 shows a 1D-TOCSY spectrum of reb M2.

FIG. 39 shows a 1D-TOCSY spectrum of reb M2.

FIG. 40 shows an HPLC (CAD) analysis.

FIG. 41 shows an HPLC (CAD) analysis.

FIG. 42 shows an HPLC (CAD) analysis.

FIG. 43 shows an HPLC (CAD) analysis.

FIG. 44 shows an HPLC (CAD) analysis.

FIG. 45 shows an HPLC (CAD) analysis.

FIG. 46 shows an HPLC (CAD) analysis.

FIG. 47 shows an HPLC (CAD) analysis.

FIG. 48 shows an HPLC (CAD) analysis.

FIG. 49 shows an HPLC (CAD) analysis.

FIG. 50 shows an HPLC (CAD) analysis.

FIG. 51 shows an HPLC (CAD) analysis.

FIG. 52 shows an HPLC (CAD) analysis.

FIG. 53 shows an LCMS chromatogram.

FIG. 54 shows an LCMS chromatogram.

FIG. 55 shows an LCMS chromatogram.

FIG. 56 shows an LCMS chromatogram.

FIG. 57 shows a reaction profile.

FIG. 58 shows an HPLC (CAD) analysis.

FIG. 59 shows an HPLC (CAD) analysis.

FIG. 60 shows an HPLC (CAD) analysis.

FIG. 61 shows an HPLC (CAD) analysis.

FIG. 62 shows an HPLC (CAD) analysis.

FIG. 63 shows an LCMS chromatogram.

FIG. 64 shows an HPLC (CAD) analysis.

FIG. 65 shows an HPLC (CAD) analysis.

FIG. 66 shows an HPLC (CAD) analysis.

FIG. 67 shows an HPLC (CAD) analysis.

FIG. 68 shows an HPLC (CAD) analysis.

FIG. 69 shows the results of an HPLC analysis.

### DETAILED DESCRIPTION

The present invention provides a biocatalytic process for preparing a composition comprising a target steviol glycoside by contacting a starting composition, comprising an organic substrate, with a microorganism and/or biocatalyst, thereby producing a composition comprising a target steviol glycoside.

One object of the invention is to provide an efficient biocatalytic method for preparing steviol glycosides, particularly stevioside, reb E, reb A, reb D, reb D2, reb M, and reb M2 from various starting compositions.

As used herein, “biocatalysis” or “biocatalytic” refers to the use of natural or genetically engineered biocatalysts, such as cells, protein enzymes, to perform single or multiple step chemical transformations on organic compounds. Biocatalysis include fermentation, biosynthesis and biotransformation processes. Both, isolated enzyme and whole-cell biocatalysis methods, using biocatalysts in free as well as immobilized forms, are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviolmonoside, steviolbioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside M2, rebaudioside D, rebaudioside D2, rebaudioside N, rebaudioside O, synthetic steviol glycosides, e.g. enzymatically glucosylated steviol glycosides and combinations thereof.

### Chemical Structures of Steviol and its Glycosides

Chemical structures of steviol and its glycosides

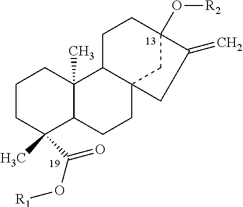

| Compound | R₁ | R₂ |
|---|---|---|
| Steviol | H | H |
| Steviolmonoside | H | β-Glc |
| Steviol monoglucosyl ester | β-Glc | H |
| Rubusoside | β-Glc | β-Glc |
| Steviolbioside | H | β-Glc-β-Glc (2→1) |
| Stevioside | β-Glc | β-Glc-β-Glc (2→1) |
| Rebaudioside A | β-Glc | 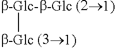 |
| Rebaudioside D | β-Glc-β-Glc (2→1) | 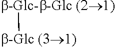 |
| Rebaudioside E | β-Glc-β-Glc (2→1) | β-Glc-β-Glc (2→1) |
| Rebaudioside M | 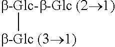 | 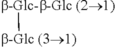 |

(Glc = glucose)

### Starting Composition

As used herein, “starting composition” refers to any composition (generally an aqueous solution) containing one or more organic compound comprising at least one carbon atom.

In one embodiment, the starting composition is selected from the group consisting of polyols and various carbohydrates.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In still another embodiment, the starting composition is sucrose.

In yet another embodiment, the starting composition is starch.

In another embodiment, the starting composition is maltodextrin.

The organic compound(s) of starting composition serve as a substrate(s) for the production of the target steviol glycoside(s), as described herein.

### Target Steviol Glycoside

The target steviol glycoside of the present method can be any steviol glycoside that can be prepared by the process disclosed herein. In one embodiment, the target steviol glycoside is selected from the group consisting of steviolmonoside, steviolbioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside M2, rebaudioside D, rebaudioside D2, rebaudioside N or rebaudioside O, or other glycoside of steviol.

In one embodiment, the target steviol glycoside is stevioside. In another embodiment, the target steviol glycoside is reb A. In still another embodiment, the target steviol glycoside is reb E. In yet another embodiment, the target steviol glycoside is reb D. In yet another embodiment, the target steviol glycoside is reb D2. In a further embodiment, the target steviol glycoside is reb M. In a still further another embodiment, the target steviol glycoside is reb M2.

The target steviol glycoside can be in any polymorphic or amorphous form, including hydrates, solvates, anhydrous or combinations thereof.

In one embodiment, the present invention is a biocatalytic process for the production of reb D.

In yet another embodiment, the present invention is a biocatalytic process for the production of reb D2.

In still another embodiment, the present invention is a biocatalytic process for the production of reb M

In a further embodiment, the present invention is a biocatalytic process for the production of reb M2.

Optionally, the method of the present invention further comprises separating the target steviol glycoside from the starting composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous basis. In one embodiment, the highly purified target steviol glycoside composition contains greater than about 90% by weight of the target steviol glycoside on an anhydrous basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dry basis.

In one embodiment, when the target steviol glycoside is reb M, the process described herein provides a composition having greater than about 90% reb M content by weight on a dry basis. In another particular embodiment, when the target steviol glycoside is reb M, the process described herein provides a composition comprising greater than about 95% reb M content by weight on a dry basis.

In another embodiment, when the target steviol glycoside is reb M2, the process described herein provides a composition having greater than about 90% reb M2 content by weight on a dry basis. In another particular embodiment, when the target steviol glycoside is reb M2, the process described herein provides a composition comprising greater than about 95% reb M2 content by weight on a dry basis.

In yet another embodiment, when the target steviol glycoside is reb D, the process described herein provides a composition greater than about 90% reb D content by weight on a dry basis. In another particular embodiment, when the target steviol glycoside is reb D, the process described herein provides a composition comprising greater than about 99% reb D content by weight on a dry basis.

In still another embodiment, when the target steviol glycoside is reb D2, the process described herein provides a composition greater than about 90% reb D2 content by weight on a dry basis. In another particular embodiment, when the target steviol glycoside is reb D2, the process described herein provides a composition comprising greater than about 95% reb D2 content by weight on a dry basis.

In a further embodiment, when the target steviol glycoside is reb A, the process described herein provides a composition comprising greater than about 90% reb A content by weight on a dry basis. In another particular embodiment, when the target steviol glycoside is reb A, the process described herein provides a composition comprising greater than about 95% reb A content by weight on a dry basis.

In a still further embodiment, when the target steviol glycoside is reb E, the process described herein provides a composition comprising greater than about 90% reb E content by weight on a dry basis. In another particular embodiment, when the target steviol glycoside is reb E, the process described herein provides a composition comprising greater than about 95% reb E content by weight on a dry basis.

In a still further embodiment, when the target steviol glycoside is reb I, the process described herein provides a composition comprising greater than about 90% reb I content by weight on a dry basis. In another particular embodiment, when the target steviol glycoside is reb I, the process described herein provides a composition comprising greater than about 95% reb I content by weight on a dry basis.

In yet a further embodiment, when the target steviol glycoside is stevioside, the process described herein provides a composition comprising greater than about 90% stevioside content by weight on a dry basis. In another particular embodiment, when the target steviol glycoside is stevioside, the process described herein provides a composition comprising greater than about 95% stevioside content by weight on a dry basis.

Microorganism and Biocatalysts

In one embodiment of present invention, a microorganism or biocatalyst is contacted with the starting composition to produce target steviol glycosides. The microorganism can be any microorganism possessing the necessary enzymes for converting the starting composition to target steviol glycosides. These enzymes are encoded within the microorganism's genome.

In one embodiment the microoganism may be, for example, E. coli, Saccharomyces sp., Aspergillus sp., Pichia sp,. Bacillus sp., Yarrowia sp. etc.

The enzymes can be located on the surface and/or inside the cell of the microorganism and/or can be secreted out in the medium by the microorganism.

The biocatalyst comprises at least one enzyme and can be whole cell suspension, crude lysate or purified enzyme.

The enzymes necessary for converting the starting composition to target steviol glycosides include the steviol biosynthesis enzymes and UDP-glycosyltransferases (UGTs). Optionally it may include UDP recycling enzyme(s). The UDP recycling enzyme can be sucrose synthase and the recycling substrate can be sucrose.

In one embodiment the steviol biosynthesis enzymes include mevalonate (MVA) pathway enzymes.

In another embodiment the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to the steviol and or steviol glycoside substrate to provide the target steviol glycoside.

In one embodiment, the microorganism is free. In another embodiment, the microorganism is immobilized. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In one embodiment the microorganism is in aqueous medium, comprising water, and various components selected form group including carbon sources, energy sources, nitrogen sources, microelements, vitamins, nucleosides, nucleoside phosphates, nucleoside diphosphates, nucleoside triphosphates, organic and inorganic salts, organic and mineral acids, bases etc. Carbon sources include glycerol, glucose, carbon dioxide, carbonates, bicarbonates. Nitrogen sources can include nitrates, nitrites, amino acids, peptides, peptones, or proteins.

In a particular embodiment, the medium comprises buffer. Suitable buffers include, but are not limited to, PIPES buffer, acetate buffer and phosphate buffer. In a particular embodiment, the medium comprises phosphate buffer.

In one embodiment, the medium can also include an organic solvent.

In one embodiment, the UDP-glucosyltransferase is any UDP-glucosyltransferase capable of adding at least one glucose unit to rubusoside, thereby producing stevioside. The UDP-glucosyltransferase may be, for example, UGT91D2.

In another embodiment, the UDP-glucosyltransferase is any UDP-glucosyltransferase capable of adding at least one glucose unit to rubusoside, thereby producing rebaudioside E. The UDP-glucosyltransferase may be, for example, UGTSL2.

In still another embodiment, the UDP-glucosyltransferase is any UDP-glucosyltransferase capable of adding at least one glucose unit to rebaudioside E, thereby producing rebaudioside D. The UDP-glucosyltransferase may be, for example, UGT76G1.

In yet embodiment, the UDP-glucosyltransferase is any UDP-glucosyltransferase capable of adding at least one glucose unit to stevioside, thereby producing rebaudioside A. The UDP-glucosyltransferase may be, for example, UGT76G1.

In a further embodiment, the UDP-glucosyltransferase is any UDP-glucosyltransferase capable of adding at least one glucose unit to rebaudioside A, thereby producing rebaudioside D and/or rebaudioside D2 and/or rebaudioside M2. The UDP-glucosyltransferase may be, for example, UGT91D2 or UGTSL2.

In another embodiment, the UDP-glucosyltransferase capable of adding at least one glucose unit to rebaudioside A is selected from the following listing of GenInfo identifier numbers, preferably from the group presented in Table 1, and more preferably the group presented in Table 2.

| | | | | | |
|---|---|---|---|---|---|
| 397567 | 30680413 | 115480946 | 147798902 | 218193594 | 225443294 |
| 454245 | 32816174 | 116310259 | 147811764 | 218193942 | 225444853 |
| 1359905 | 32816178 | 116310985 | 147827151 | 219885307 | 225449296 |
| 1685003 | 34393978 | 116788066 | 147836230 | 222615927 | 225449700 |
| 1685005 | 37993665 | 116788606 | 147839909 | 222619587 | 225454338 |
| 2191136 | 37993671 | 116789315 | 147846163 | 222623142 | 225454340 |
| 2501497 | 37993675 | 119394507 | 147855977 | 222625633 | 225454342 |
| 2911049 | 39104603 | 119640480 | 148905778 | 222625635 | 225454473 |
| 4218003 | 41469414 | 122209731 | 148905999 | 222636620 | 225454475 |
| 4314356 | 41469452 | 125526997 | 148906835 | 222636621 | 225458362 |
| 13492674 | 42566366 | 125534279 | 148907340 | 222636628 | 225461551 |
| 13492676 | 42570280 | 125534461 | 148908935 | 222636629 | 225461556 |
| 15217773 | 42572855 | 125540090 | 148909182 | 224053242 | 225461558 |
| 15217796 | 44890129 | 125541516 | 148909920 | 224053386 | 225469538 |
| 15223396 | 46806235 | 125545408 | 148910082 | 224055535 | 225469540 |
| 15223589 | 50284482 | 125547340 | 148910154 | 224056138 | 226316457 |
| 15227766 | 51090402 | 125547520 | 148910612 | 224056160 | 226492603 |
| 15230017 | 51090594 | 125554547 | 148910769 | 224067918 | 226494221 |
| 15231757 | 52839682 | 125557592 | 156138791 | 224072747 | 226495389 |
| 15234056 | 56550539 | 125557593 | 156138797 | 224080189 | 226495945 |
| 15234195 | 62734263 | 125557608 | 156138799 | 224091845 | 226502400 |
| 15234196 | 62857204 | 125559566 | 156138803 | 224094703 | 226507980 |
| 15238503 | 62857206 | 125563266 | 165972256 | 224100653 | 226531147 |
| 15239523 | 62857210 | 125571055 | 168016721 | 224100657 | 226532094 |
| 15239525 | 62857212 | 125579728 | 171674071 | 224101569 | 238477377 |
| 15239543 | 75265643 | 125588307 | 171906258 | 224103105 | 240254512 |
| 15239937 | 75285934 | 125589492 | 183013901 | 224103633 | 242032615 |
| 15240305 | 75288884 | 125599469 | 183013903 | 224103637 | 242032621 |
| 15240534 | 77550661 | 125601477 | 186478321 | 224109218 | 242038423 |
| 15982889 | 77556148 | 126635837 | 187373030 | 224114583 | 242043290 |
| 18086351 | 82791223 | 126635845 | 187373042 | 224116284 | 242044836 |
| 18418378 | 83778990 | 126635847 | 190692175 | 224120552 | 242051252 |
| 18418380 | 89953335 | 126635863 | 194701936 | 224121288 | 242056217 |
| 18418382 | 110741436 | 126635867 | 195620060 | 224121296 | 242056219 |
| 19743740 | 110743955 | 126635883 | 209954691 | 224121300 | 242056663 |
| 19911201 | 115438196 | 126635887 | 209954719 | 224130358 | 242059339 |
| 20149064 | 115438785 | 133874210 | 209954725 | 224140703 | 242059341 |
| 20260654 | 115441237 | 133874212 | 209954733 | 224143404 | 242060922 |
| 21435782 | 115454819 | 145358033 | 210063105 | 224143406 | 242067411 |
| 21553613 | 115456047 | 147772508 | 210063107 | 224144306 | 242067413 |
| 21593514 | 115457492 | 147776893 | 212275846 | 224285244 | 242076258 |
| 22759895 | 115459312 | 147776894 | 216296854 | 225431707 | 242076396 |
| 23955910 | 115464719 | 147776895 | 217074506 | 225435532 | 242084750 |
| 26452040 | 115471069 | 147786916 | 218185693 | 225436321 | 242091005 |
| 28393204 | 115471071 | 147798900 | 218187075 | 225440041 | 242095206 |
| 30679796 | 115474009 | 147798901 | 218189427 | 225441116 | 242345159 |
| 242345161 | 297724601 | 326492035 | 356523945 | 357140904 | 359486938 |
| 255536859 | 297725463 | 326493430 | 356523957 | 357165849 | 359487055 |
| 255538228 | 297728331 | 326500410 | 356523959 | 357165852 | 359488135 |
| 255541676 | 297738632 | 326506816 | 356523961 | 357168415 | 359488708 |
| 255547075 | 297745347 | 326507826 | 356523963 | 357437837 | 359493630 |
| 255552620 | 297745348 | 326508394 | 356524387 | 357442755 | 359493632 |
| 255552622 | 297795735 | 326509445 | 356524403 | 357442757 | 359493634 |
| 255555343 | 297796253 | 326511261 | 356527181 | 357445729 | 359493636 |
| 255555361 | 297796257 | 326511866 | 356533209 | 357445731 | 359493815 |
| 255555363 | 297796261 | 326512412 | 356533852 | 357445733 | 359495856 |
| 255555365 | 297797587 | 326517673 | 356534718 | 357446799 | 359495858 |
| 255555369 | 297798502 | 326518800 | 356535480 | 357446805 | 359495869 |
| 255555373 | 297799226 | 326521124 | 356542996 | 357452779 | 359495871 |
| 255555377 | 297805988 | 326525567 | 356543136 | 357452781 | 359497638 |
| 255556812 | 297807499 | 326525957 | 356543932 | 357452783 | 359807261 |
| 255556818 | 297809125 | 326526607 | 356549841 | 357452787 | 374256637 |
| 255563008 | 297809127 | 326527141 | 356549843 | 357452789 | 377655465 |
| 255564074 | 297811403 | 326530093 | 356554358 | 357452791 | 378405177 |
| 255564531 | 297820040 | 326534036 | 356554360 | 357452797 | 378829085 |
| 255572878 | 297821483 | 326534312 | 356558606 | 357452799 | 387135070 |
| 255577901 | 297825217 | 332071132 | 356560333 | 357470367 | 387135072 |
| 255583249 | 297832276 | 339715876 | 356560599 | 357472193 | 387135078 |
| 255583253 | 297832280 | 342306012 | 356560749 | 357472195 | 387135092 |
| 255583255 | 297832518 | 342306016 | 356566018 | 357474295 | 387135094 |
| 255585664 | 297832520 | 343457675 | 356566169 | 357474493 | 387135098 |
| 255585666 | 297840825 | 343457677 | 356566173 | 357474497 | 387135100 |
| 255634688 | 297840827 | 350534960 | 356567761 | 357474499 | 387135134 |
| 255644801 | 297847402 | 356498085 | 356574704 | 357490035 | 387135136 |
| 255645821 | 297849372 | 356499771 | 356576401 | 357493567 | 387135174 |
| 255647456 | 300078590 | 356499777 | 356577660 | 357497139 | 387135176 |
| 255648275 | 300669727 | 356499779 | 357114993 | 357497581 | 387135184 |
| 260279126 | 302142947 | 356501328 | 357115447 | 357497671 | 387135186 |
| 260279128 | 302142948 | 356502523 | 357115451 | 357500579 | 387135188 |
| 261343326 | 302142950 | 356503180 | 357115453 | 357504663 | 387135190 |
| 283132367 | 302142951 | 356503184 | 357116080 | 357504691 | 387135192 |
| 283362112 | 302765302 | 356503295 | 357116928 | 357504699 | 387135194 |
| 289188052 | 302796334 | 356504436 | 357117461 | 357504707 | 387135282 |
| 295841350 | 302811470 | 356504523 | 357117463 | 357505859 | 387135284 |
| 296088529 | 302821107 | 356504765 | 357117829 | 357510851 | 387135294 |
| 296090415 | 302821679 | 356511113 | 357117839 | 357516975 | 387135298 |
| 296090524 | 319759260 | 356515120 | 357125059 | 359477003 | 387135300 |
| 296090526 | 319759266 | 356517088 | 357126015 | 359477998 | 387135302 |
| 297599503 | 320148814 | 356520732 | 357134488 | 359478043 | 387135304 |
| 297601531 | 326489963 | 356522586 | 357135657 | 359478286 | 387135312 |
| 297611791 | 326490273 | 356522588 | 357138503 | 359484299 | 387135314 |
| 297722841 | 326491131 | 356522590 | 357139683 | 359486936 | 387135316 |
| 387135318 | 449440433 | 460376293 | 460413408 | 462423864 | 475546199 |
| 387135320 | 449445896 | 460378310 | 460416351 | 470101924 | 475556485 |
| 387135322 | 449446454 | 460380744 | 462394387 | 470102280 | 475559699 |
| 387135324 | 449447657 | 460381726 | 462394433 | 470102858 | 475578293 |
| 387135326 | 449449002 | 460382093 | 462394557 | 470104211 | 475591753 |
| 387135328 | 449449004 | 460382095 | 462395646 | 470104264 | 475593742 |
| 388493506 | 449449006 | 460382754 | 462395678 | 470104266 | 475612072 |
| 388495496 | 449451379 | 460384935 | 462396388 | 470106317 | 475622476 |
| 388498446 | 449451589 | 460384937 | 462396389 | 470106357 | 475622507 |
| 388499220 | 449451591 | 460385076 | 462396419 | 470115448 | 475623787 |
| 388502176 | 449451593 | 460385872 | 462396542 | 470130404 | 482550481 |
| 388517521 | 449453712 | 460386018 | 462397507 | 470131550 | 482550499 |
| 388519407 | 449453714 | 460389217 | 462399998 | 470136482 | 482550740 |
| 388521413 | 449453716 | 460394872 | 462400798 | 470136484 | 482550999 |
| 388827901 | 449453732 | 460396139 | 462401217 | 470136488 | 482552352 |
| 388827903 | 449457075 | 460397862 | 462402118 | 470136492 | 482554970 |
| 388827907 | 449467555 | 460397864 | 462402237 | 470137933 | 482555336 |
| 388827909 | 449468742 | 460398541 | 462402284 | 470137937 | 482555478 |
| 388827913 | 449495638 | 460403139 | 462402416 | 470140422 | 482556454 |
| 393887637 | 449495736 | 460403141 | 462404228 | 470140426 | 482557289 |
| 393887646 | 449499880 | 460403143 | 462406358 | 470140908 | 482558462 |
| 393887649 | 449502786 | 460403145 | 462408262 | 470141232 | 482558508 |
| 393990627 | 449503471 | 460405998 | 462409325 | 470142008 | 482558547 |
| 397746860 | 449503473 | 460407578 | 462409359 | 470142010 | 482561055 |
| 397789318 | 449515857 | 460407590 | 462409777 | 470142012 | 482561555 |
| 413924864 | 449518643 | 460409128 | 462411467 | 470143607 | 482562795 |
| 414590349 | 449519559 | 460409134 | 462414311 | 470143939 | 482562850 |
| 414590661 | 449522783 | 460409136 | 462414416 | 470145404 | 482565074 |
| 414591157 | 449524530 | 460409459 | 462414476 | 473923244 | 482566269 |
| 414879558 | 449524591 | 460409461 | 462415526 | 474114354 | 482566296 |
| 414879559 | 449528823 | 460409463 | 462415603 | 474143634 | 482566307 |
| 414879560 | 449528825 | 460409465 | 462415731 | 474202268 | 482568689 |
| 414888074 | 449534021 | 460409467 | 462416307 | 474299266 | 482570049 |
| 431812559 | 460365546 | 460410124 | 462416920 | 474363119 | 482570572 |
| 449432064 | 460366882 | 460410126 | 462416922 | 474366157 | 482575121 |
| 449432066 | 460369823 | 460410128 | 462416923 | 474429346 | |
| 449433069 | 460369829 | 460410130 | 462416924 | 475432777 | |
| 449436944 | 460369831 | 460410132 | 462417401 | 475473002 | |
| 449438665 | 460369833 | 460410134 | 462419769 | 475489790 | |
| 449438667 | 460370755 | 460410213 | 462420317 | 475511330 | |
| 449440431 | 460374714 | 460411200 | 462423366 | 475516200 | |

TABLE 1

| GI number | Accession | Origin |
|---|---|---|
| 190692175 | ACE87855.1 | Stevia rebaudiana |
| 41469452 | AAS07253.1 | Oryza sativa |
| 62857204 | BAD95881.1 | Ipomoea nil |
| 62857206 | BAD95882.1 | Ipomoea purperea |
| 56550539 | BAD77944.1 | Bellis perennis |
| 115454819 | NP_001051010.1 | Oryza sativa Japonica Group |
| 115459312 | NP_001053256.1 | Oryza sativa Japonica Group |
| 115471069 | NP_001059133.1 | Oryza sativa Japonica Group |
| 115471071 | NP_001059134.1 | Oryza sativa Japonica Group |
| 116310985 | CAH67920.1 | Oryza sativa Indica Group |
| 116788066 | ABK24743.1 | Picea sitchensis |
| 122209731 | Q2V6J9.1 | Fragaria × ananassa |
| 125534461 | EAY81009.1 | Oryza sativa Indica Group |
| 125559566 | EAZ05102.1 | Oryza sativa Indica Group |
| 125588307 | EAZ28971.1 | Oryza sativa Japonica Group |
| 148907340 | ABR16806.1 | Picea sitchensis |
| 148910082 | ABR18123.1 | Picea sitchensis |
| 148910612 | ABR18376.1 | Picea sitchensis |
| 15234195 | NP_194486.1 | Arabidopsis thaliana |
| 15239523 | NP_200210.1 | Arabidopsis thaliana |
| 15239937 | NP_196793.1 | Arabidopsis thaliana |
| 1685005 | AAB36653.1 | Nicotiana tabacum |
| 183013903 | ACC38471.1 | Medicago truncatula |
| 186478321 | NP_172511.3 | Arabidopsis thaliana |
| 187373030 | ACD03249.1 | Avena strigosa |
| 194701936 | ACF85052.1 | Zea mays |
| 19743740 | AAL92461.1 | Solanum lycopersicum |
| 212275846 | NP_001131009.1 | Zea mays |
| 222619587 | EEE55719.1 | Oryza sativa Japonica Group |
| 224055535 | XP_002298527.1 | Populus trichocarpa |
| 224101569 | XP_002334266.1 | Populus trichocarpa |
| 224120552 | XP_002318358.1 | Populus trichocarpa |
| 224121288 | XP_002330790.1 | Populus trichocarpa |
| 225444853 | XP_002281094 | Vitis vinifera |
| 225454342 | XP_002275850.1 | Vitis vinifera |
| 225454475 | XP_002280923.1 | Vitis vinifera |
| 225461556 | XP_002285222 | Vitis vinifera |
| 225469540 | XP_002270294.1 | Vitis vinifera |
| 226495389 | NP_001148083.1 | Zea mays |
| 226502400 | NP_001147674.1 | Zea mays |
| 238477377 | ACR43489.1 | Triticum aestivum |
| 240254512 | NP_565540.4 | Arabidopsis thaliana |
| 2501497 | Q43716.1 | Petunia × hybrida |
| 255555369 | XP_002518721.1 | Ricinus communis |
| 26452040 | BAC43110.1 | Arabidopsis thaliana |
| 296088529 | CBI37520.3 | Vitis vinifera |
| 297611791 | NP_001067852.2 | Oryza sativa Japonica Group |
| 297795735 | XP_002865752.1 | Arabidopsis lyrata subsp. lyrata |
| 297798502 | XP_002867135.1 | Arabidopsis lyrata subsp. lyrata |
| 297820040 | XP_002877903.1 | Arabidopsis lyrata subsp. lyrata |
| 297832276 | XP_002884020.1 | Arabidopsis lyrata subsp. lyrata |
| 302821107 | XP_002992218.1 | Selaginella moellendorffii |
| 30680413 | NP_179446.2 | Arabidopsis thaliana |
| 319759266 | ADV71369.1 | Pueraria montana var. lobata |
| 326507826 | BAJ86656.1 | Hordeum vulgare subsp. Vulgare |
| 343457675 | AEM37036.1 | Brassica rapa subsp. oleifera |
| 350534960 | NP_001234680.1 | Solanum lycopersicum |
| 356501328 | XP_003519477.1 | Glycine max |
| 356522586 | XP_003529927.1 | Glycine max |
| 356535480 | XP_003536273.1 | Glycine max |
| 357445733 | XP_003593144.1 | Medicago truncatula |
| 357452783 | XP_003596668.1 | Medicago truncatula |
| 357474493 | XP_003607531.1 | Medicago truncatula |
| 357500579 | XP_003620578.1 | Medicago truncatula |
| 357504691 | XP_003622634.1 | Medicago truncatula |
| 359477998 | XP_003632051.1 | Vitis vinifera |
| 359487055 | XP_002271587 | Vitis vinifera |
| 359495869 | XP_003635104.1 | Vitis vinifera |
| 387135134 | AFJ52948.1 | Linum usitatissimum |
| 387135176 | AFJ52969.1 | Linum usitatissimum |
| 387135192 | AFJ52977.1 | Linum usitatissimum |
| 387135282 | AFJ53022.1 | Linum usitatissimum |
| 387135302 | AFJ53032.1 | Linum usitatissimum |
| 387135312 | AFJ53037.1 | Linum usitatissimum |
| 388519407 | AFK47765.1 | Medicago truncatula |
| 393887646 | AFN26668.1 | Barbarea vulgaris subsp. arcuata |
| 414888074 | DAA64088.1 | Zea mays |
| 42572855 | NP_974524.1 | Arabidopsis thaliana |
| 449440433 | XP_004137989.1 | Cucumis sativus |
| 449446454 | XP_004140986.1 | Cucumis sativus |
| 449449004 | XP_004142255.1 | Cucumis sativus |
| 449451593 | XP_004143546.1 | Cucumis sativus |
| 449515857 | XP_004164964.1 | Cucumis sativus |
| 460382095 | XP_004236775.1 | Solanum lycopersicum |
| 460409128 | XP_004249992.1 | Solanum lycopersicum |
| 460409461 | XP_004250157.1 | Solanum lycopersicum |
| 460409465 | XP_004250159.1 | Solanum lycopersicum |
| 462396388 | EMJ02187.1 | Prunus persica |
| 462402118 | EMJ07675.1 | Prunus persica |
| 462409359 | EMJ14693.1 | Prunus persica |
| 462416923 | EMJ21660.1 | Prunus persica |
| 46806235 | BAD17459.1 | Oryza sativa Japonica Group |
| 470104266 | XP_004288529.1 | Fragaria vesca subsp. vesca |
| 470142008 | XP_004306714.1 | Fragaria vesca subsp. vesca |
| 475432777 | EMT01232.1 | Aegilops tauschii |
| 51090402 | BAD35324.1 | Oryza sativa Japonica Group |

TABLE 2

| | | | Internal |
|---|---|---|---|
| GI number | Accession | Origin | reference |
| 460409128 | XP.004249992.1 | Solanum lycopersicum | UGTSL |
| 460386018 | XP.004238697.1 | Solanum lycopersicum | — |
| 460409134 | XP.004249995.1 | Solanum lycopersicum | — |
| 460410132 | XP.004250485.1 | Solanum lycopersicum | UGTSL2 |
| 460410130 | XP.004250484.1 | Solanum lycopersicum | — |
| 460410128 | XP.004250483.1 | Solanum lycopersicum | — |
| 460378310 | XP.004234916.1 | Solanum lycopersicum | — |
| 209954733 | BAG80557.1 | Lycium barbarum | UGTLB |
| 209954725 | BAG80553.1 | Lycium barbarum | — |

In yet another embodiment, the UDP-glucosyltransferase is any UDP-glucosyltransferase capable of adding at least one glucose unit to rebaudioside D to form rebaudioside M and/or rebaudioside M2. The UDP-glucosyltransferase may be, for example, UGT76G1.

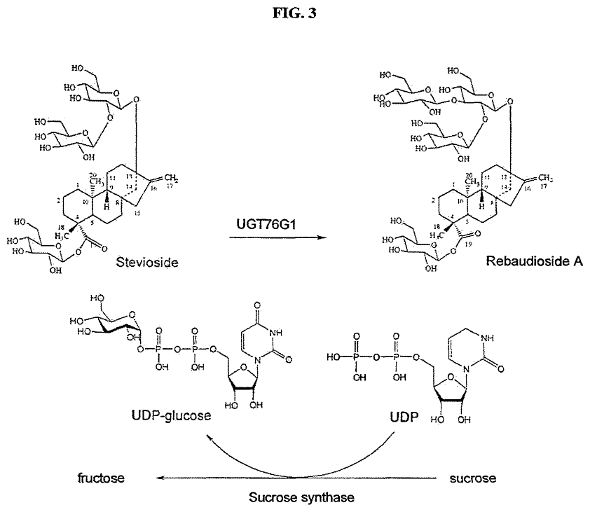

Optionally, the method of the present invention further comprises recycling UDP to provide UDP-glucose. In one embodiment, the method comprises recycling UDP by providing a recycling catalyst, i.e., a biocatalyst capable of UDP-glucose overproduction, and a recycling substrate, such that the conversion of the substrate steviol glycoside to the target steviol glycoside is carried out using catalytic amounts of UDP-glucosyltransferase and UDP-glucose (FIG. 3).

In one embodiment, the UDP-glucose recycling catalyst is sucrose synthase.

In one embodiment, the recycling substrate is sucrose.

In one embodiment the biocatalyst comprises more than one UDP-glucosyltransferase.

In embodiment the biocatalyst comprises more than one UDP-glucosyltransferase and UDP-glucose recycling catalyst.

The target steviol glycoside is optionally purified from the resulting composition.

Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

Compounds and Methods

The present invention also provides isolated and highly purified reb D2. Reb D2 is an isomer of reb D and has the following structure:

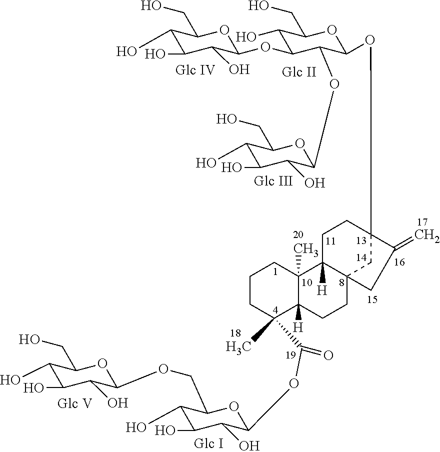

### 13-[(2-O-β-D-glucopyranosyl-3-O-β-D-glucopyranosyl-β-D-glucopyranosyl)oxy]ent-kaur-16-en-19-oic acid-[(6-O-β-D-glucopyranosyl-β-D-glucopyranosyl) ester]

In another embodiment, the present invention provides reb D2 having a purity greater than about 95% by weight on an anhydrous basis, such as, for example, greater than about 96% by weight, greater than about 97% by weight, greater than about 98% by weight or greater than about 99% by weight.

In still another embodiment, the present invention provides reb D2 having a purity greater than about 95% by weight in a steviol glycoside mixture, such as, for example, greater than about 96% by weight, greater than about 97% by weight, greater than about 98% by weight or greater than about 99% by weight.

The present invention also provides compositions comprising reb D2.

In one embodiment, the present invention provides a method for preparing reb D2 comprising:
a. contacting a starting composition comprising reb A with an enzyme capable of transforming reb A to reb D2, UDP-glucose, and optionally UDP-glucose recycling enzymes, to produce a composition comprising reb D2; and
b. isolating the composition comprising reb D2.

In some embodiments, the enzyme capable of transforming reb A to reb D2 is a UDP-glucosyltransferase, such as, for example, UGT91D2, UGTSL, UGTSL_Sc, UGTSL2 (GI No. 460410132 version XP_004250485.1), GI No. 460409128 (UGTSL) version XP_004249992.1, GI No. 115454819 version NP_001051010.1, GI No. 187373030, version ACD03249.1. GI No. 222619587 version EEE55719.1, GI No. 297795735 version XP_002865752.1 or EUGT11.

The enzyme capable of transforming reb A to reb D2 can be immobilized or in a recombinant microorganism.

In one embodiment, the enzyme is immobilized. In another embodiment, the enzyme is in a recombinant microorganism.

In one embodiment, the microorganism is free. In another embodiment, the microorganism is immobilized. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

Suitable microorganisms include, but are not limited to, E. coli, Saccharomyces sp., Aspergillus sp., Pichia sp., Bacillus sp., Yarrowia sp.

In one embodiment the microorganism is in an aqueous medium, comprising water, and various components selected form group including carbon sources, energy sources, nitrogen sources, microelements, vitamins, nucleosides, nucleoside phosphates, nucleoside diphosphates, nucleoside triphosphates, organic and inorganic salts, organic and mineral acids, bases etc. Carbon sources include glycerol, glucose, carbon dioxide, carbonates, bicarbonates. Nitrogen sources can include nitrates, nitrites, amino acids, peptides, peptones, or proteins.

In a particular embodiment, the medium comprises buffer. Suitable buffers include, but are not limited to, PIPES buffer, acetate buffer and phosphate buffer. In a particular embodiment, the medium comprises phosphate buffer.

In one embodiment the medium can also include an organic solvent.

In a particular embodiment, the enzyme is a UDP-glucosyltransferase capable of transforming reb A to reb D2.

In a more particular embodiment, the enzyme is selected from UGT91D2, UGTSL, UGTSL_Sc, UGTSL2 (GI No. 460410132 version XP_004250485.1), GI No. 460409128 (UGTSL) verison XP_004249992.1, GI No. 115454819 version NP_001051010.1, GI No. 187373030, version ACD03249.1. GI No. 222619587 version EEE55719.1, GI No. 297795735 version XP_002865752.1 or EUGT11 and UGTs having substantial (>85%) sequence identity to these.

In a still more particular embodiment, the enzyme is UGTSL2 or its improved variant produced by directed evolution and having higher activity.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the reaction.

Isolation of reb D2 from the reaction medium can be achieved by any suitable method to provide a composition comprising reb D2. Suitable methods include, but are not limited to, lysis, crystallization, separation by membranes, centrifugation, extraction (liquid or solid phase), chromatographic separation, HPLC (preparative or analytical) or a combination of such methods. In a particular embodiment, isolation can be achieved by lysis and centrifugation.

In some embodiments, isolation may result in a reb D2 purity less than about 95% by weight on an anhydrous basis, and the composition may contain, e.g., steviol glycosides and/or residual reaction products. The composition comprising reb D2 can be further purified to provide highly purified reb D2, i.e. reb D2 having a purity greater than about 95% by weight on an anhydrous basis. In some embodiments, the compositions comprising reb D2 can be further purified to provide reb D2 having a purity greater than about 96%, greater than about 97%, greater than about 98% or greater than about 99% by weight on an anhydrous basis.

Purification can be affected by any means known to one of skill in the art including, but not limited to, crystallization, separation by membranes, centrifugation, extraction (liquid or solid phase), chromatographic separation, HPLC (preparative or analytical) or a combination of such methods. In a particular embodiment, HPLC is used to purify reb D2. In a more particular embodiment, semi-preparative HPLC is used to purify reb D2.

For example, a two-step semi-preparative HPLC purification can be used. The first step utilizes a C18 column with a mobile phase containing A (25% MeCN in water) and B (30% MeCN in water) with the following gradient:

| Time (min) | % A | % B |
|---|---|---|
| 0.0-5.0 | 100 | 0 |
| 20 | 20 | 80 |
| 25 | 20 | 80 |
| 30 | 100 | 0 |

The secondary step utilizes the same column and conditions, but with only an isocratic mobile phase: 20% MeCN in water.

Those of skill in the art will recognize that the particular column, mobile phases, injection volumes and other HPLC parameters can vary.

In one embodiment, the present invention provides isolated and highly purified reb M2. Reb M2 is an isomer of reb M and has the following structure:

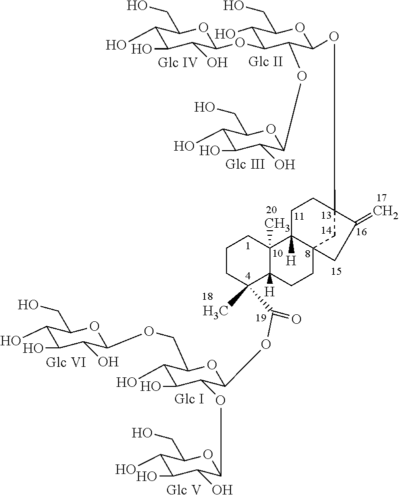

### (13-[(2-O-β-D-glucopyranosyl-3-O-β-D-glucopyranosyl-β-D-glucopyranosyl)oxy]ent-kaur-16-en-19-oic acid-[(2-O-β-D-glucopyranosyl-6-O-β-D-glucopyranosyl-β-D-glucopyranosyl) ester])

In another embodiment, the present invention provides reb M2 having a purity greater than about 95% by weight on an anhydrous basis, such as, for example, greater than about 96% by weight, greater than about 97% by weight, greater than about 98% by weight or greater than about 99% by weight.

In still another embodiment, the present invention provides reb M2 having a purity greater than about 95% by weight in a steviol glycoside mixture, such as, for example, greater than about 96% by weight, greater than about 97% by weight, greater than about 98% by weight or greater than about 99% by weight.

In yet another embodiment, the present invention provides reb M2 having a purity greater than about 95% by weight in a stevia extract, such as, for example, greater than about 96% by weight, greater than about 97% by weight, greater than about 98% by weight or greater than about 99% by weight.

The present invention also provides compositions comprising reb M2.

It has been found that reb M2 is produced during biotransformation of reb A to reb D. As noted above, biotransformation of reb A to reb D also produces reb D2. Accordingly, in one embodiment, the present invention provides a method for preparing reb M2 comprising:
a. contacting a starting composition comprising reb A and/or reb D2 with an enzyme capable of transforming reb A and/or reb D2 to reb M2, UDP-glucose, and optionally UDP-glucose recycling enzymes to produce a composition comprising reb M2; and
b. isolating a composition comprising reb M2.

Not wishing to be bound by theory, it is currently believed that the pathway begins with transformation of reb A to reb D2, followed by transformation of reb D2 to reb M2.

Accordingly, In one embodiment, the present invention provides a method for preparing reb M2 comprising:
a. contacting a starting composition comprising reb D2 with an enzyme capable of transforming reb D2 to reb M2, UDP-glucose, and optionally UDP-glucose
recycling enzymes to produce a composition comprising reb M2; and
b. isolating a composition comprising reb M2.

In yet another embodiment, a method for preparing reb M2 comprises:
a. contacting a starting composition comprising reb A with an enzyme capable of transforming reb A to reb D2, UDP-glucose, and optionally UDP-glucose recycling enzymes to produce a composition comprising reb D2;
b. optionally, isolating a composition comprising reb D2;
c. contacting the composition comprising reb D2 with an enzyme capable of transforming reb D2 to reb M2, UDP-glucose, and optionally UDP-glucose recycling enzymes to produce a composition comprising rebM2; and
d. isolating a composition comprising reb M2.

The enzyme can be a UDP-glucosyltransferase, such as, for example, UGT91D2, UGTSL, UGTSL_Sc, UGTSL2 (GI No. 460410132 version XP_004250485.1), GI No. 460409128 (UGTSL) version XP_004249992.1, GI No. 115454819 version NP_001051010.1, GI No. 187373030, version ACD03249.1. GI No. 222619587 version EEE55719.1, GI No. 297795735 version XP_002865752.1 or EUGT11.

The enzyme can be immobilized or in a recombinant microorganism.

In one embodiment, the enzyme is immobilized. In another embodiment, the enzyme is in a recombinant microorganism.

In one embodiment, the microorganism is free. In another embodiment, the microorganism is immobilized. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

Suitable microorganisms include, but are not limited to, E. coli, Saccharomyces sp., Aspergillus sp., Pichia sp., Bacillus sp., Yarrowia sp.

In one embodiment the microorganism is in aqueous medium, comprising water, and various components selected form group including carbon sources, energy sources, nitrogen sources, microelements, vitamins, nucleosides, nucleoside phosphates, nucleoside diphosphates, nucleoside triphosphates, organic and inorganic salts, organic and mineral acids, bases etc. Carbon sources include glycerol, glucose, carbon dioxide, carbonates, bicarbonates. Nitrogen sources can include nitrates, nitrites, amino acids, peptides, peptones, or proteins.

In a particular embodiment, the medium comprises buffer. Suitable buffers include, but are not limited to, PIPES buffer, acetate buffer and phosphate buffer. In a particular embodiment, the medium comprises phosphate buffer.

In one embodiment the medium can also include an organic solvent.

In a particular embodiment, the enzyme is a UDP-glucosyltransferase capable of transforming reb A and/or reb D2 to reb M2 and is contained in E. coli.

In a more particular embodiment, the enzyme is selected from UGT91D2, UGTSL, UGTSL_Sc, UGTSL2 (GI No. 460410132 version XP_004250485.1), GI No. 460409128 (UGTSL) verison XP_004249992.1, GI No. 115454819 version NP_001051010.1, GI No. 187373030, version ACD03249.1. GI No. 222619587 version EEE55719.1, GI No. 297795735 version XP_002865752.1 or EUGT11.

In a still more particular embodiment, the enzyme is UGTSL2 or its improved variant produced by directed evolution and having higher activity.

In one embodiment, the target steviol glycoside reb M2 can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the reaction.

Isolation of reb M2 from the reaction medium can be achieved by any suitable method to provide a composition comprising reb M2. Suitable methods include, but are not limited to, lysis, crystallization, separation by membranes, centrifugation, extraction (liquid or solid phase), chromatographic separation, HPLC (preparative or analytical) or a combination of such methods. In a particular embodiment, isolation can be achieved by lysis and centrifugation.

In some embodiments, isolation may result in a reb M2 purity less than about 95% by weight on an anhydrous basis, and the composition may contain, e.g., steviol glycosides and/or residual reaction products.

The composition comprising reb M2 can be further purified to provide highly purified reb M2, i.e. reb M2 having a purity greater than about 95% by weight on an anhydrous basis. In some embodiments, the compositions comprising reb M2 can be further purified to provide reb M2 having a purity greater than about 96%, greater than about 97%, greater than about 98% or greater than about 99% by weight on an anhydrous basis.

Purification can be affected by any means known to one of skill in the art including, but not limited to, crystallization, separation by membranes, centrifugation, extraction (liquid or solid phase), chromatographic separation, HPLC (preparative or analytical) or a combination of such methods. In a particular embodiment, HPLC is used to purify reb M2. In a more particular embodiment, semi-preparative HPLC is used to purify reb M2.

For example, a two-step semi-preparative HPLC purification can be used. The first step utilizes a C18 column with a mobile phase containing A (25% MeCN in water) and B (30% MeCN in water) with the following gradient:

| Time (min) | % A | % B |
|---|---|---|
| 0.0-5.0 | 100 | 0 |
| 20 | 20 | 80 |
| 25 | 20 | 80 |
| 30 | 100 | 0 |

The secondary step utilizes the same column and conditions, but with only an isocratic mobile phase: 20% MeCN in water.

Those of skill in the art will recognize that the particular column, mobile phases, injection volumes and other HPLC parameters can vary.

Purified steviol glycosides, prepared in accordance with the present invention, may be used in a variety of consumable products including, but not limited to, foods, beverages, pharmaceutical compositions, tobacco products, nutraceutical compositions, oral hygiene compositions, and cosmetic compositions.

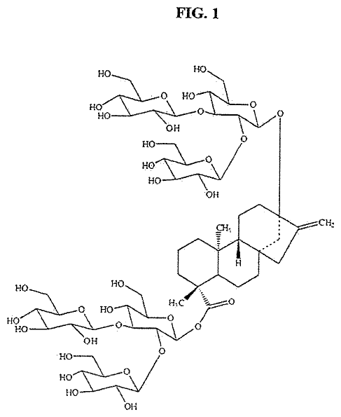

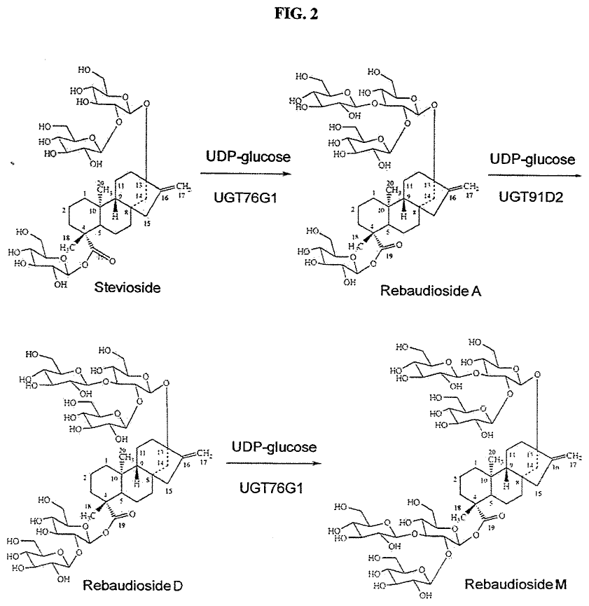

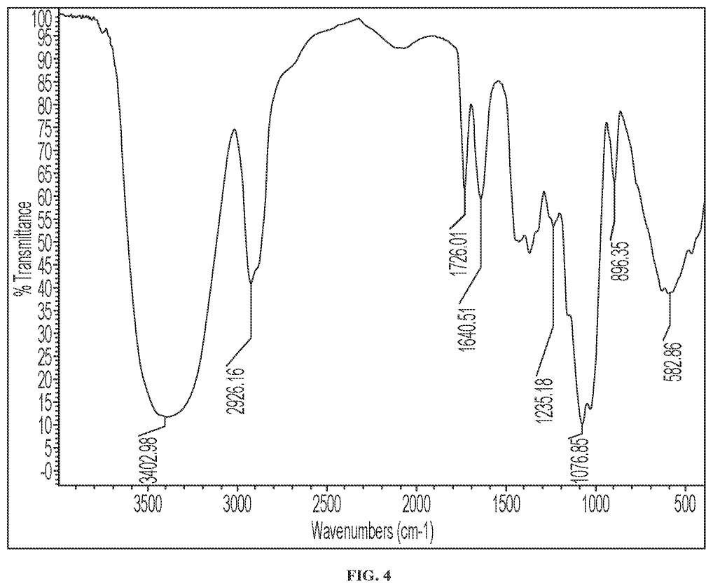

The high purity reb M obtained in this invention, having a molecular weight of 1291.29, a molecular formula of C₅₆H₉₀O₃₃, CAS registry number 1220616-44-3, and the structure presented in FIG. 1, is in the form of a white and odorless powder. The compound is about 200 times sweeter than sugar when compared to a 10% sucrose solution. The infrared absorption spectrum is shown in FIG. 4.

Other properties of the pure reb M compound include a melting point of 249-250° C., and a specific rotation of [α]_{D}²⁵−19.0° in 50% ethanol (C=1.0). The solubility of reb Min water is around 0.3%, and increases with an increase in temperature.

Reb M is soluble in diluted solutions of methanol, ethanol, n-propanol, and isopropanol. However, it is insoluble in acetone, benzene, chloroform, and ether.

Reb M obtained in accordance with the present invention is heat and pH-stable.

Highly purified target glycoside(s) particularly, reb D, reb D2, reb M and/or reb M2 obtained according to this invention can be used “as-is” or in combination with at least one sweetener, flavor, food ingredient and/or combination thereof.

Non-limiting examples of flavors include lime, lemon, orange, fruit, banana, grape, pear, pineapple, mango, berry, bitter almond, cola, cinnamon, sugar, cotton candy and vanilla flavors and/or combination thereof.

Non-limiting examples of other food ingredients include at least one selected from flavors, acidulants, organic and amino acids, coloring agents, bulking agents, modified starches, gums, texturizers, preservatives, antioxidants, emulsifiers, stabilizers, thickeners and gelling agents and/or combination thereof.

Highly purified target glycoside(s) particularly, reb D, reb D2, reb M and/or reb M2 obtained according to this invention can be prepared in various polymorphic forms, including but not limited to hydrates, solvates, anhydrous, amorphous forms and/or combination thereof.

Highly purified target steviol glycoside(s), particularly, reb D, reb D2, reb M and/or reb M2 obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target steviol glycoside(s), particularly, reb D, reb D2, reb M and/or reb M2 as a sweetening compound may be employed as the sole sweetener, or it may be used together with at least one naturally occurring high intensity sweeteners such as stevioside, reb A, reb B, reb C, reb D, reb E, reb F, steviolbioside, dulcoside A, rubusoside, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, camosiflosides, cyclocarioside, pterocaryosides, polypodoside A, brazilin, hemandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside and/or combination thereof.

In a particular embodiment, reb D2 and/or reb M2 can be used together in a sweetener composition comprising a compound selected from the group consisting of reb A, reb B, reb D, NSF-02, Mogroside V, erythritol and/or combinations thereof.

Highly purified target steviol glycoside(s), particularly, reb D, reb D2, reb M and/or reb M2 may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and the like.

Moreover, highly purified target steviol glycoside(s), particularly, reb D, reb D2, reb M and/or reb M2 can be used in combination with natural sweetener suppressors such as gymnemic acid, hodulcin, ziziphin, lactisole, and others. Reb D, reb D2, reb M and/or reb M2 may also be combined with various umami taste enhancers. Reb D, reb D2, reb M and/or reb M2 can be mixed with umami tasting and sweet amino acids such as glutamate, aspartic acid, glycine, alanine, threonine, proline, serine, glutamate, lysine and tryptophan.

Highly purified target steviol glycoside(s), particularly, reb D, reb D2, reb M can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

Highly purified target steviol glycoside(s), particularly, reb D, reb D2, reb M and/or reb M2 may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly, reb D, reb D2, reb M and/or reb M2 may be combined with reduced calorie sweeteners such as D-tagatose, allulose, allose, L-sugars, L-sorbose, L-arabinose, and others.

Highly purified target steviol glycoside(s), particularly, reb D, reb D2, reb M and/or reb M2 may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, Psicose, turanose, allulose, allose, D-tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose com syrups, coupling sugars, and soybean oligosaccharides. Additionally, the carbohydrates as used herein may be in either the D- or L-configuration.

Highly purified target steviol glycoside(s), particularly, reb D, reb D2, reb M and/or reb M2 obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly, reb D, reb D2, reb M and/or reb M2 obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly, reb D, reb D2, reb M and/or reb M2 can be used as a sweetener not only for drinks, foodstuffs, and other products dedicated for human consumption, but also in animal feed and fodder with improved characteristics.

Examples of consumable products in which highly purified target steviol glycoside(s), particularly, reb D, reb D2, reb M and/or reb M2 may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s), particularly, reb D, reb D2, reb M and/or reb M2 obtained in this invention may be used in dry or liquid forms. In one embodiment, a tabletop sweetener comprising reb D2 is provided. In another embodiment, a tabletop sweetener comprising reb M2 is provided.

The highly purified target steviol glycoside can be added before or after heat treatment of food products. The amount of the highly purified target steviol glycoside(s), particularly, reb D, reb D2, reb M and/or reb M2 depends on the purpose of usage. As discussed above, it can be added alone or in combination with other compounds.

The present invention is also directed to sweetness enhancement in beverages using reb D2. The present invention is also directed to sweetness enhancement in beverages using reb M2. Accordingly, the present invention provides a beverage comprising a sweetener and reb D2 and/or reb M2 as a sweetness enhancer, wherein reb D2 and/or reb M2 is present in a concentration at or below their respective sweetness recognition thresholds.

As used herein, the term “sweetness enhancer” refers to a compound capable of enhancing or intensifying the perception of sweet taste in a composition, such as a beverage. The term “sweetness enhancer” is synonymous with the terms “sweet taste potentiator,” “sweetness potentiator,” “sweetness amplifier,” and “sweetness intensifier.”

The term “sweetness recognition threshold concentration,” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.5% to about 12% by weight, such as, for example, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.5% of about 10%, such as for example, from about 2% to about 8%, from about 3% to about 7% or from about 4% to about 6% by weight. In a particular embodiment, the sweetener is present in the beverage in an amount from about 0.5% to about 8% by weight. In another particular embodiment, the sweetener is present in the beverage in an amount from about 2% to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, L-ribose, D-tagatose, L-glucose, L-fucose, L-arbinose, D-turanose, D-leucrose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In yet another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a beverage comprising a sweetener comprising providing a beverage comprising a sweetener and adding a sweetness enhancer selected from reb D2, reb M2 or a combination thereof, wherein reb D2 and reb M2 are present in a concentration at or below their sweetness recognition thresholds.

Addition of reb D2 and/or reb M2 in a concentration at or below the sweetness recognition threshold to a beverage containing a sweetener may increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

The following examples illustrate preferred embodiments of the invention for the preparation of highly purified target steviol glycoside(s), particularly, reb D, reb D2, reb M and/or reb M2. It will be understood that the invention is not limited to the materials, proportions, conditions and procedures set forth in the examples, which are only illustrative.

### Example 1

### In-Vivo Production of UGT76G1

NcoI and NdeI restriction sides were added to the original nucleic sequence as described in Genbank accession no. AAR06912.1. After codon optimization the following nucleic sequence was obtained (SEQ ID 1):

CCATGGCCCATATGGAAAACAAAACCGAAACCACCGTTCGTCGTCGTCGC
CGTATTATTCTGTTTCCGGTTCCGTTTCAGGGTCATATTAATCCGATTCT
GCAGCTGGCAAATGTGCTGTATAGCAAAGGTTTTAGCATTACCATTTTTC
ATACCAATTTTAACAAACCGAAAACCAGCAATTATCCGCATTTTACCTTT
CGCTTTATTCTGGATAATGATCCGCAGGATGAACGCATTAGCAATCTGCC
GACACATGGTCCGCTGGCAGGTATGCGTATTCCGATTATTAACGAACATG
GTGCAGATGAACTGCGTCGTGAACTGGAACTGCTGATGCTGGCAAGCGAA
GAAGATGAAGAAGTTAGCTGTCTGATTACCGATGCACTGTGGTATTTTGC
ACAGAGCGTTGCAGATAGCCTGAATCTGCGTCGTCTGGTTCTGATGACCA
GCAGCCTGTTTAACTTTCATGCACATGTTAGCCTGCCGCAGTTTGATGAA
CTGGGTTATCTGGATCCGGATGATAAAACCCGTCTGGAAGAACAGGCAAG
CGGTTTTCCGATGCTGAAAGTGAAAGATATCAAAAGCGCCTATAGCAATT
GGCAGATTCTGAAAGAAATTCTGGGCAAAATGATTAAACAGACCAAAGCA
AGCAGCGGTGTTATTTGGAATAGCTTTAAAGAACTGGAAGAAAGCGAACT
GGAAACCGTGATTCGTGAAATTCCGGCACCGAGCTTTCTGATTCCGCTGC
CGAAACATCTGACCGCAAGCAGCAGCAGCCTGCTGGATCATGATCGTACC
GTTTTTCAGTGGCTGGATCAGCAGCCTCCGAGCAGCGTTCTGTATGTTAG
CTTTGGTAGCACCAGCGAAGTTGATGAAAAAGATTTTCTGGAAATTGCCC
GTGGTCTGGTTGATAGCAAACAGAGCTTTCTGTGGGTTGTTCGTCCGGGT
TTTGTTAAAGGTAGCACCTGGGTTGAACCGCTGCCGGATGGTTTTCTGGG
TGAACGTGGTCGTATTGTTAAATGGGTTCCGCAGCAAGAAGTTCTGGCAC
ACGGCGCAATTGGTGCATTTTGGACCCATAGCGGTTGGAATAGCACCCTG
GAAAGCGTTTGTGAAGGTGTTCCGATGATTTTTAGCGATTTTGGTCTGGA
TCAGCCGCTGAATGCACGTTATATGAGTGATGTTCTGAAAGTGGGTGTGT
ATCTGGAAAATGGTTGGGAACGTGGTGAAATTGCAAATGCAATTCGTCGT
GTTATGGTGGATGAAGAAGGTGAATATATTCGTCAGAATGCCCGTGTTCT
GAAACAGAAAGCAGATGTTAGCCTGATGAAAGGTGGTAGCAGCTATGAAA
GCCTGGAAAGTCTGGTTAGCTATATTAGCAGCCTGTAATAACTCGAG

After synthesis of the gene and subcloning into pET30A+ vector using NdeI and XhoI cloning sites, the UGT76G1_pET30a+ plasmid was introduced in E. coli B121(DE3) and E. coli EC100 by electroporation. The obtained cells were grown in petri-dishes in the presence of Kanamycin and suitable colonies were selected and allowed to grow in liquid LB medium (erlenmeyer flasks). Glycerol was added to the suspension as cryoprotectant and 400 μL aliquots were stored at −20° C. and at −80° C.

The storage aliquots of E. coli BL21(DE3) containing the pET30A+_UGT76G1 plasmid were thawed and added to 30 mL of LBGKP medium (20 g/L Luria Broth Lennox; 50 mM PIPES buffer pH 7.00; 50 mM Phosphate buffer pH 7.00; 2.5 g/L glucose and 50 mg/L of Kanamycin). This culture was allowed to shake at 135 rpm at 30° C. for 8 h.

The production medium contained 60 g/L of overnight express instant TB medium (Novagen), 10 g/L of glycerol and 50 mg/L of Kanamycin. The medium was allowed to stir at 20° C. while taking samples to measure the OD and pH. The cultures gave significant growth and a good OD was obtained. After 40 h, the cells were harvested by centrifugation and frozen to yield 12.7 g of cell wet weight.

Lysis was performed by addition of Bugbuster Master mix (Novagen) and the lysate was recovered by centrifugation and kept frozen. Activity tests were performed with thawed lysate.

### Example 2

### In-Vitro Production of UGT76G1

The S30 T7 High Yield Protein expression system kit from Promega was used. 4 g of UGT76G1_pET30a+ plasmid from E. coli EC100 was mixed with 80 μL of S30 premix plus and 72 μL of S30 T7 extract was added. Nuclease-free water was added in order to obtain a total volume of 200 μL and the resulting solution was incubated for 2 h at 30° C. 180 μL was used in the catalytic test reaction.

### Example 3

### In-Vitro Production of UGT91D2

NcoI and NdeI restriction sides were added to the original nucleic sequence as described in Genbank accession no. ACE87855.1. After codon optimization the following nucleic sequence was obtained (SEQ ID 2):

CCATGGCACATATGGCAACCAGCGATAGCATTGTTGATGATCGTAAACAG
CTGCATGTTGCAACCTTTCCGTGGCTGGCATTTGGTCATATTCTGCCGTA
TCTGCAGCTGAGCAAACTGATTGCAGAAAAAGGTCATAAAGTGAGCTTTC
TGAGCACCACCCGTAATATTCAGCGTCTGAGCAGCCATATTAGTCCGCTG
ATTAATGTTGTTCAGCTGACCCTGCCTCGTGTTCAAGAACTGCCGGAAGA
TGCCGAAGCAACCACCGATGTTCATCCGGAAGATATTCCGTATCTGAAAA
AAGCAAGTGATGGTCTGCAGCCGGAAGTTACCCGTTTTCTGGAACAGCAT
AGTCCGGATTGGATCATCTATGATTATACCCATTATTGGCTGCCGAGCAT
TGCAGCAAGCCTGGGTATTAGCCGTGCACATTTTAGCGTTACCACCCCGT
GGGCAATTGCATATATGGGTCCGAGCGCAGATGCAATGATTAATGGTAGT
GATGGTCGTACCACCGTTGAAGATCTGACCACCCCTCCGAAATGGTTTCC
GTTTCCGACCAAAGTTTGTTGGCGTAAACATGATCTGGCACGTCTGGTTC
CGTATAAAGCACCGGGTATTAGTGATGGTTATCGTATGGGTCTGGTTCTG
AAAGGTAGCGATTGTCTGCTGAGCAAATGCTATCATGAATTTGGCACCCA
GTGGCTGCCGCTGCTGGAAACCCTGCATCAGGTTCCGGTTGTTCCGGTGG
GTCTGCTGCCTCCGGAAGTTCCGGGTGATGAAAAAGATGAAACCTGGGTT
AGCATCAAAAAATGGCTGGATGGTAAACAGAAAGGTAGCGTGGTTTATGT
TGCACTGGGTAGCGAAGTTCTGGTTAGCCAGACCGAAGTTGTTGAACTGG
CACTGGGTCTGGAACTGAGCGGTCTGCCGTTTGTTTGGGCATATCGTAAA
CCGAAAGGTCCGGCAAAAAGCGATAGCGTTGAACTGCCGGATGGTTTTGT
TGAACGTACCCGTGATCGTGGTCTGGTTTGGACCAGCTGGGCACCTCAGC
TGCGTATTCTGAGCCATGAAAGCGTTTGTGGTTTTCTGACCCATTGTGGT
AGCGGTAGCATTGTGGAAGGTCTGATGTTTGGTCATCCGCTGATTATGCT
GCCGATTTTTGGTGATCAGCCGCTGAATGCACGTCTGCTGGAAGATAAAC
AGGTTGGTATTGAAATTCCGCGTAATGAAGAAGATGGTTGCCTGACCAAA
GAAAGCGTTGCACGTAGCCTGCGTAGCGTTGTTGTTGAAAAAGAAGGCGA
AATCTATAAAGCCAATGCACGTGAACTGAGCAAAATCTATAATGATACCA
AAGTGGAAAAAGAATATGTGAGCCAGTTCGTGGATTATCTGGAAAAAAAC
ACCCGTGCAGTTGCCATTGATCACGAAAGCTAATGACTCGAG

After synthesis of the gene and subcloning into pET30A+ vector using NeoI and XhoI cloning sites, the UGT91D2_pET30a+ plasmid was introduced into E. coli EC100 by electroporation. The obtained cells were grown in the presence of Kanamycin and suitable colonies were selected and allowed to grow in liquid LB medium (erlenmeyer flasks). Glycerol was added to the suspension as cryoprotectant and 400 μL aliquots were stored at −20° C. and at −80° C.

The S30 T7 High Yield Protein expression system kit from Promega was used for the in-vitro synthesis of the protein.

4 μg of UGT91D2_pET30a+ plasmid was mixed with 80 μL of S30 premix plus and 72 μL of S30 T7 extract was added. Nuclease-free water was added in order to obtain a total volume of 200 μL and the resulting solution was incubated for 2 h at 30° C. 5 μL was used for SDS-page analysis while the remaining 45 μL was used in the catalytic test reaction.

### Example 4

Catalytic Reaction with In-Vivo Produced UGT76G1

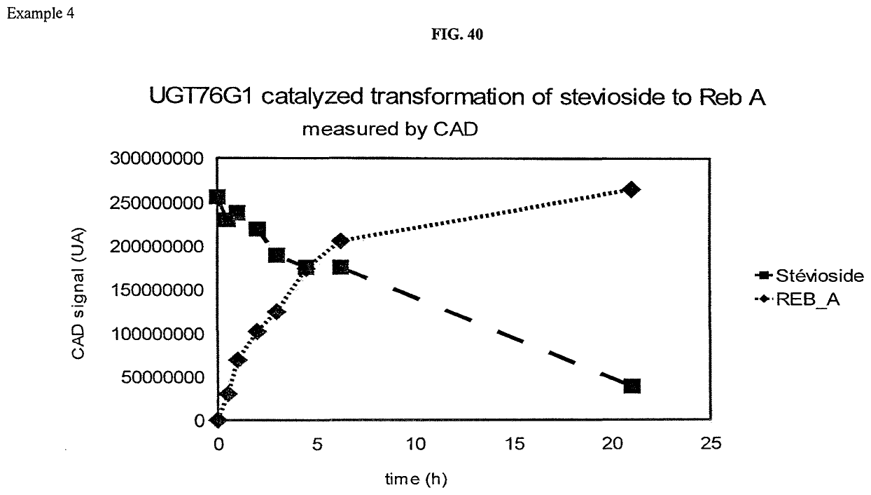

The total volume of the reaction was 5.0 mL with the following composition: 50 mM sodium phosphate buffer pH 7.2, 3 mM MgCl₂, 2.5 mM UDP-glucose, 0.5 mM Stevioside and 500 μL of UGT76G1 thawed lysate. The reactions were run at 30° C. on an orbitary shaker at 135 rpm. For each sample, 460 μL of the reaction mixture was quenched with 40 μL of 2N H₂SO₄ and 420 μL of methanol/water (6/4). The samples were immediately centrifuged and kept at 10° C. before analysis by HPLC (CAD). HPLC indicated almost complete conversion of stevioside to rebaudioside A as seen in FIG. 40.

### Example 5

Catalytic Reaction with In-Vitro Produced UGT91D2

The total volume of the reaction was 0.5 mL with the following composition: 50 mM sodium phosphate buffer pH 7.2, 3 mM MgCl₂, 3.8 mM UDP-glucose, 0.1 mM Rebaudioside A and 180 μL of in-vitro produced UGT91D2. The reactions were run at 30° C. on an orbitary shaker at 135 rpm. For each sample, 450 μL of reaction mixture was quenched with 45 μL of 2N H₂SO₄ and 405 μL of 60% MeOH. After centrifugation, the supernatant was analyzed by HPLC (CAD). HPLC indicated a 4.7% conversion of rebaudioside A to rebaudioside D after 120 h.

### Example 6

Catalytic Reaction with In-Vitro Produced UGT76G1

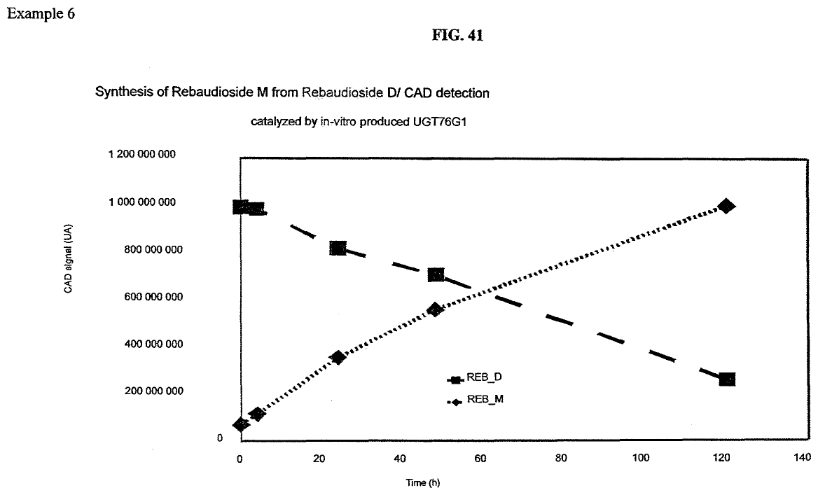

The total volume of the reaction was 2 mL with the following composition: 50 mM sodium phosphate buffer pH 7.2, 3 mM MgCl₂, 3.8 mM UDP-glucose, 0.5 mM Rebaudioside D and 180 μL of in-vitro produced UGT76G1. The reactions were run at 30° C. on an orbitary shaker at 135 rpm. For each sample, 400 μL of reaction mixture was quenched with 40 μL of 2N H₂SO₄ and 360 μL of 60% MeOH. After centrifugation, the supernatant was analyzed by HPLC (CAD). HPLC indicated 80% conversion of rebaudioside D to rebaudioside M after 120 h as seen in FIG. 41.
For examples 7 to 12, the following abbreviations were used:
LBGKP medium: 20 g/L Luria Broth Lennox; 50 mM PIPES buffer pH 7.00; 50 mM
Phosphate buffer pH 7.00; 2.5 g/L glucose and 50 mg/L of Kanamycin or Ampicillin
LB medium: (20 g/L Luria Broth Lennox)

### Example 7

Preparation and Activity of UGT76G1 Prepared by pET30a+ Plasmid and BL21 (DE3) Expression Strain

The pET30a+_UGT76G1 plasmid was transformed into BL21(DE3) expression strain (Lucigen E. Cloni® EXPRESS Electrocompetent Cells). The obtained cells were grown on LB Agar medium in petri-dishes in the presence of Kanamycin. Suitable colonies were selected and allowed to grow in liquid LBGKP medium containing Kanamycin. Glycerol was added and 400 μL aliquots were stored at −20° C. and at −80° C.

A storage aliquot was thawed and added to 30 mL of LBGKP medium. This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Kanamycin. The medium was allowed to stir at 20° C. while taking samples to measure the OD (600 nm) and pH. After 40 h, the cells were harvested by centrifugation and frozen. The obtained cell wet weight was 10.58 g.

3.24 g of obtained pellet was lysed by addition of 8.1 mL of “Bugbuster Master mix” (Novagen, reference 71456) and 3.5 mL of water. The lysate was recovered by centrifugation and kept frozen.

### Example 8

Preparation and Activity of UGT76G1 Prepared by pET30a+ Plasmid and Tuner (DE3) Expression Strain

The pET30a+_UGT76G1 plasmid was transformed into Tuner (DE3) expression strain (Novagen Tunere™ (DE3) Competent cells) by heat shock treatment. The obtained cells were grown on LB Agar medium in petri-dishes in the presence of Kanamycin. Suitable colonies were selected and allowed to grow in liquid LBGKP medium containing Kanamycin). Glycerol was added and 400 μL aliquots were stored at −20° C. and at −80° C.

A storage aliquot was thawed and added to 100 mL of LB medium containing 50 mg/L of Kanamycin. This culture allowed to shake at 30° C. for 15 h. 4.4 mL of this culture was used to inoculate 200 mL of production medium containing LB. This medium was allowed to stir at 37° C. until an OD (600 nm) of 0.9 was obtained, after which 400 μL of a 100 mM IPTG solution was added and the medium was allowed to stir at 30° C. for 4 h. The cells were harvested by centrifugation and frozen. The obtained cell wet weight was 1.38 g.

The obtained pellet was lysed by addition of 4.9 mL of “Bugbuster Master mix” (Novagen, reference 71456) and 2.1 mL of water. The lysate was recovered by centrifugation and kept frozen.

### Example 9

Preparation and activity of UGT76G1 prepared by pMAL plasmid and BL21 expression strain After subcloning the synthetic UGT76G1 gene into the pMAL plasmid using Ndel and Sal1 cloning sites, the pMAL_UGT76G1 plasmid was transformed into BL21 expression strain (New England Biolabs BL21 Competent E. coli) by heat shock treatment. The obtained cells were grown on LB Agar medium in petri-dishes in the presence of Ampicillin. Suitable colonies were selected and allowed to grow in liquid LBGKP medium containing Ampicillin). Glycerol was added and 400 μL aliquots were stored at −20° C. and at −80° C.

A storage aliquot was thawed and added to 30 mL of LBGKP medium. This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Ampicillin. The medium was allowed to stir at 20° C. while taking samples to measure the OD and pH. After 40 h, the cells were harvested by centrifugation and frozen. The obtained cell wet weight was 5.86 g.

2.74 g of obtained pellet was lysed by addition of 9.6 mL of “Bugbuster Master Mix” (Novagen, reference 71456) and 4.1 mL of water. The lysate was recovered by centrifugation and kept frozen.

### Example 10

Preparation and Activity of UGT76G1 Prepared by pMAL Plasmid and ArcticExpress Expression Strain

The pMAL_UGT76G1 plasmid was transformed into ArticExpress expression strain (Agilent ArcticExpress competent cells) by heat shock treatment. The obtained cells were grown on LB Agar medium in petri-dishes in the presence of Ampicillin and Geneticin. Suitable colonies were selected and allowed to grow in liquid LBGKP medium containing of Ampicillin and Geneticin. Glycerol was added and 400 μL aliquots were stored at −20° C. and at −80° C.

A storage aliquot was thawed and added to 30 mL of LBGKP medium (containing Ampicillin and Geneticin). This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Ampicillin. The medium was allowed to stir at 12° C. while taking samples to measure the OD (600 nm) and pH. After 68 h, the cells were harvested by centrifugation and frozen. The obtained cell wet weight was 8.96 g.

2.47 g of the obtained pellet was lysed by addition of 8.73 mL of “Bugbuster Master Mix” (Novagen, reference 71456) and 3.79 mL of water. The lysate was recovered by centrifugation and kept frozen.

### Example 11

Preparation and activity of UGT76G1 prepared by pCOLDIII plasmid and ArcticExpress expression strain After subcloning the synthetic UGT76G1 gene into the pCOLDIII plasmid using Ndel and Xhol cloning sites, the pCOLDIII_UGT76G1 plasmid was transformed into ArcticExpress expression strain (Agilent ArcticExpress competent cells) by heat shock treatment. The obtained cells were grown on LB Agar medium in petri-dishes in the presence of Ampicillin and Geneticin. Suitable colonies were selected and allowed to grow in liquid LBGKP medium containing Ampicillin and Geneticin. Glycerol was added and 400 μL aliquots were stored at −20° C. and at −80° C.

A storage aliquot was thawed and added to 30 mL of LBGKP medium (containing Ampicillin and Geneticin). This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Kanamycin. The medium was allowed to stir at 12° C. while taking samples to measure the OD (600 nm) and pH. After 63 h, the cells were harvested by centrifugation and frozen. The obtained cell wet weight was 6.54 g.

2.81 g of the obtained pellet was lysed by addition of 9.8 mL of “Bugbuster Master Mix” (Novagen, reference 71456) and 4.2 mL of water. The lysate was recovered by centrifugation and kept frozen.

### Example 12

Preparation and Activity of UGT76G1 Prepared by pCOLDIII Plasmid and Origami2 (DE3) Expression Strain

The pCOLDIII_UGT76G1 plasmid was transformed into Origami2 (DE3) expression strain (Novagen Origami™2 (DE3) Competent Cells) by heat shock treatment. The obtained cells were grown on LB Agar medium in petri-dishes in the presence of Ampicillin. Suitable colonies were selected and allowed to grow in liquid LBGKP medium containing Ampicillin. Glycerol was added and 400 μL aliquots were stored at −20° C. and at −80° C.

A storage aliquot was thawed and added to 30 mL of LBGKP medium (containing Ampicillin). This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Kanamycin. The medium was allowed to stir at 12° C. while taking samples to measure the OD (600 nm) and pH. After 68 h, the cells were harvested by centrifugation and frozen. The obtained cell wet weight was 2.53 g.

1.71 g of the obtained pellet was lysed by addition of 6.0 mL of “Bugbuster Master mix” (Novagen, reference 71456) and 1.9 mL of water. The lysate was recovered by centrifugation and kept frozen.

### Example 13

### Determination of Activity

Activity tests were performed on a 5 mL scale with 500 μL of thawed lysate for the transformation of Stevioside to Rebaudioside A and Rebaudioside D to Rebaudioside M using 0.5 mM of substrate, 2.5 mM of UDP-Glucose and 3 mM MgCl₂ in 50 mM Sodium Phosphate buffer at pH 7.2. Samples were taken and analyzed by HPLC. The results for the different preparations of UGT76G1 are summarized in the following table.

| | | | Transformation activity* | |
|---|---|---|---|---|
| | | Expression | Stevioside to | Rebaudioside D to |
| Example | Plasmid | strain | Rebaudioside A | Rebaudioside M |
| 7 | pET30a+ | BL21 (DE3) | 29 U mL⁻¹ | 0.31 U mL⁻¹ |
| 8 | pET30a+ | Tuner (DE3) | 33 U mL⁻¹ | 0.40 U mL⁻¹ |
| 9 | pMAL | BL21 | 20 U mL⁻¹ | 0.15 U mL⁻¹ |
| 10 | pMAL | ArticExpress | 15 U mL⁻¹ | 0.25 U mL⁻¹ |
| 11 | pCOLDIII | ArticExpress | 15 U mL⁻¹ | 0.11 U mL⁻¹ |
| 12 | pCOLDIII | Origami2 (DE3) | 37 U mL⁻¹ | 0.20 U mL⁻¹ |

*Note
The activities for the transformation of Stevioside and Rebaudioside M are mentioned per mL of lysate. 1 U will transform 1 μmol of substrate in 1 hour at 30° C. and pH 7.2

### Example 14

### 50 mL Scale Reaction for the Transformation of Rebaudioside D to Rebaudioside M

5 mL of the lysate of Example 12 was used to transform Rebaudioside D to Rebaudioside M on a 50 mL scale. The reaction medium consisted of 50 mM Sodium Phosphate buffer pH 7.2, 3 mM of MgCl₂, 2.5 mM of UDP-Glucose and 0.5 mM of Rebaudioside D. After allowing the reaction to be shaken at 30° C. for 90 h. 50 mL of ethanol was added and the resulting mixture was allowed to stir at −20° C. for 1 h. After centrifugation at 5000 g for 10 min. the supernatant was purified via ultrafiltration (Vivaflow MWCO 30000). 78 mL of permeate was obtained and the 9 mL of retentate was diluted with 9 mL of ethanol and resubjected to Ultrafiltration (Vivaflow MWCO 30000). Another 14 mL of filtrate was obtained, which was combined with the first permeate. The combined permeates were concentrated under reduced pressure at 30° C. until 32 mL of a clear solution was obtained.

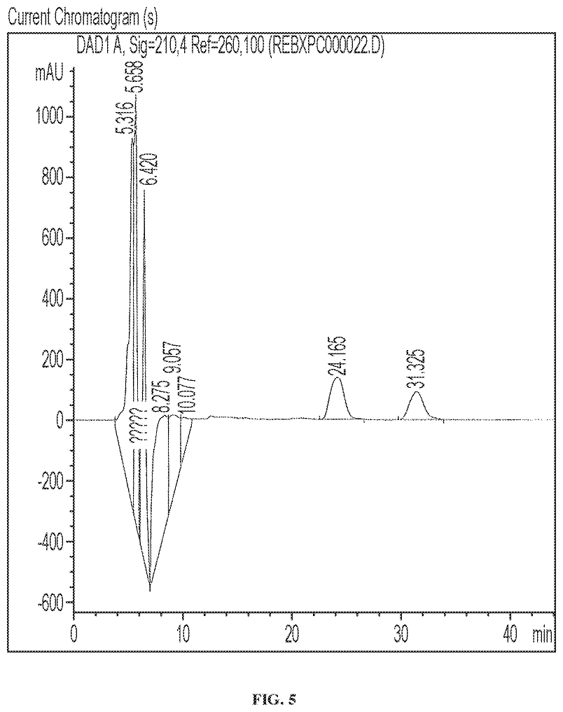

The HPLC trace of the product mixture is shown in FIG. 5. HPLC was carried out on an Agilent 1200 series equipped with a binary pump, auto sampler, and thermostat column compartment. The method was isocratic, with a mobile phase composed of 70% water (0.1% formic acid): 30% acetonitrile. The flow rate was 0.1 μL/min. The column used was Phenomenex Prodigy 5 ODS (3) 100 A; 250×2 mm. The column temperature was maintained at 40° C. The injection volume was 20-40 μl.

### Example 15

Preparation of UGT91D2 Using pMAL Plasmid and BL21 Expression Strain

After subcloning the synthetic UGT91D2 gene into the pMAL plasmid using Ndel and Sal1 cloning sites, the pMAL_UGT91D2 plasmid was transformed into BL21 expression strain (New England Biolabs BL21 Competent E. coli) by heat shock treatment. The obtained cells were grown on LB Agar medium in petri-dishes in the presence of Ampicillin. Suitable colonies were selected and allowed to grow in liquid LBGKP medium containing Ampicillin). Glycerol was added and 400 μL aliquots were stored at −20° C. and at −80° C.

A storage aliquot was thawed and added to 30 mL of LBGKP medium. This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Ampicillin. The medium was allowed to stir at 20° C. while taking samples to measure the OD and pH. After 40 h, the cells were harvested by centrifugation and frozen. The obtained cell wet weight is 12.32 g.

2.18 g of obtained pellet was lysed by addition of 7.7 mL of “Bugbuster Master Mix” (Novagen, reference 71456) and 3.2 mL of water. The lysate was recovered by centrifugation and used directly for activity testing.

### Example 16

Preparation of UGT91D2 Using pMAL Plasmid and ArcticExpress Expression Strain

The pMAL_UGT91D2 plasmid was transformed into ArcticExpress expression strain (Agilent ArcticExpress competent cells) by heat shock treatment. The obtained cells were grown on LB Agar medium in petri-dishes in the presence of Ampicillin and Geneticin. Suitable colonies were selected and allowed to grow in liquid LBGKP medium containing Ampicillin and Geneticin. Glycerol was added and 400 μL aliquots were stored at −20° C. and at −80° C.

A storage aliquot was thawed and added to 30 mL of LBGKP medium (containing Ampicillin and Geneticin). This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Ampicillin. The medium was allowed to stir at 20° C. for 16 h. followed by another 50 h. at 12° C. while taking samples to measure the OD (600 nm) and pH. The cells were harvested by centrifugation and frozen. The obtained cell wet weight is 15.77 g.

2.57 g of the obtained pellet was lysed by addition of 9.0 mL of “Bugbuster Master Mix” (Novagen, reference 71456) and 3.8 mL of water. The lysate was recovered by centrifugation and used directly for activity testing.

### Example 17

Preparation of UGT91D2 using pET30a+ plasmid and Tuner (DE3) expression strain The pET30a+_UGT91D2 plasmid was transformed into Tuner (DE3) expression strain (Novagen Tuner™ (DE3) Competent cells) by heat shock treatment. The obtained cells were grown on LB Agar medium in petri-dishes in the presence of Kanamycin. Suitable colonies were selected and allowed to grow in liquid LBGKP medium (containing Kanamycin). Glycerol was added and 400 μL aliquots were stored at −20° C. and at −80° C.

A storage aliquot was thawed and added to 100 mL of LB medium containing 50 mg/L of Kanamycin. This culture allowed to shake at 30° C. for 15 h. 6.2 mL of this culture was used to inoculate 500 mL of production medium containing LB. This medium was allowed to stir at 37° C. until an OD (600 nm) of 0.9 was obtained after which 500 μL of a 100 mM IPTG solution was added (IPTG concentration in medium is 100 μM) and the medium was allowed to stir at 30° C. for 4 h, the cells were harvested by centrifugation and frozen. The obtained cell wet weight is 4.02 g.

1.92 g of the obtained pellet was lysed by addition of 6.8 mL of “Bugbuster Master mix” (Novagen, reference 71456) and 2.8 mL of water. The lysate was recovered by centrifugation and tested directly for activity.

### Example 18

Preparation of UGT91D2 Using pET30a+ Plasmid and ArcticExpress Expression Strain

The pET30a+_UGT91D2 plasmid was transformed into ArcticExpress (DE3) expression strain (Agilent ArcticExpress competent cells) by heat shock treatment. The obtained cells were grown on LB Agar medium in petri-dishes in the presence of Kanamycin and Geneticin. Suitable colonies were selected and allowed to grow in liquid LBGKP medium containing of Kanamycin and Geneticin. Glycerol was added and 400 μL aliquots were stored at −20° C. and at −80° C.

A storage aliquot was thawed and added to 30 mL of LBGKP medium (containing Kanamycin and Geneticin). This culture was allowed to shake at 30° C. for 8 h. and subsequently used to inoculate 400 mL of production medium containing 60 g/L of “Overnight express instant TB medium” (Novagen, reference 71491-5), 10 g/L of glycerol and 50 mg/L of Ampicillin. The medium was allowed to stir at 20° C. for 16 h. followed by another 50 h. at 12° C. while taking samples to measure the OD (600 nm) and pH. After 60 h, the cells were harvested by centrifugation and frozen. The obtained cell wet weight is 16.07 g.

3.24 g of the obtained pellet was lysed by addition of 11.4 mL of “Bugbuster Master Mix” (Novagen, reference 71456) and 4.8 mL of water. The lysate was recovered by centrifugation and used directly for activity testing.

### Example 19

### Determination of Activity of In-Vivo Preparations of UGT91D2

Activity tests were performed at 5 mL scale with 1000 μL of lysate for the transformation of Rubusoside to Stevioside using 0.5 mM of substrate, 2.5 mM of UDP-Glucose and 3 mM MgCl₂ in 50 mM Sodium Phosphate buffer at pH 7.2. Samples were taken and analyzed by HPLC. The results for the different preparations of UGT91D2 are summarized in the following table.

| | | | Transformation activity* |
|---|---|---|---|
| Example | Plasmid | Expression strain | Rubusoside to Stevioside |
| 15 | pMAL | BL21 | 9 mU mL⁻¹ |
| 16 | pMAL | ArcticExpress | 60 mU mL⁻¹ |
| 17 | pET30a+ | Tuner (DE3) | 28 mU mL⁻¹ |
| 18 | pET30a+ | ArcticExpress (DE3) | 21 mU mL⁻¹ |

*Note:
The activities are mentioned per mL of lysate. 1 U will transform 1 μmol of substrate in 1 hour at 30° C. and pH 7.2

### Example 20

### Other Enzymes for Rebaudioside a to Rebaudioside D Conversion

The following genes of UDP-glucosyltransferases were identified from public databases, synthesized by DNA2.0 and subsequently subcloned in pET30a+ vector.

| | | | Internal | Conversion |
|---|---|---|---|---|
| Microplate | Position | Gene Name | reference | RebA to RebD |
| C908201 | A1 | gi115454819_NP_001051010.1 | S115N01 A1 | Active |
| C908201 | G2 | gi187373030_ACD03249.1 | S115N01 G2 | Active |
| C908201 | A7 | gi460409128_XP_004249992.1 | S115N05 A7 | Active |
| C912666 | E1 | gi222619587_EEE55719.1 | S115N06 E1 | Active |
| C912666 | C2 | gi297795735_XP_002865752.1 | S115N06 C2 | Active |

The aminoacid sequences are as follows:

>gi|115454819|ref|NP_001051010.1|
Os03g0702500 [Oryza sativa Japonica Group]
SEQ ID 3
MDDAHSSQSPLHVVIFPWLAFGHLLPCLDLAERLAARGHRVSFVSTPRNL
ARLPPVRPELAELVDLVALPLPRVDGLPDGAEATSDVPFDKFELHRKAFD
GLAAPFSAFLDTACAGGKRPDWVLADLMHHWVALASQERGVPCAMILPCS
AAVVASSAPPTESSADQREAIVRSMGTAAPSFEAKRATEEFATEGASGVS
IMTRYSLTLQRSKLVAMRSCPELEPGAFTILTRFYGKPVVPFGLLPPRPD
GARGVSKNGKHDAIMQWLDAQPAKSVVYVALGSEAPMSADLLRELAHGLD
LAGTRFLWAMRKPAGVDADSVLPAGFLGRTGERGLVTTRWAPQVSILAHA
AVCAFLTHCGWGSVVEGLQFGHPLIMLPILGDQGPNARILEGRKLGVAVP
RNDEDGSFDRGGVAGAVRAVVVEEEGKTFFANARKLQEIVADREREERCI
DEFVQHLTSWNELKNNSDGQYP
>gi|187373030|gb|ACD03249.1| UDP-
glycosyltransferase [Avena strigosa]
SEQ ID 4
MAVKDEQQSPLHILLFPFLAPGHLIPIADMAALFASRGVRCTILTTPVNA
AIIRSAVDRANDAFRGSDCPAIDISVVPFPDVGLPPGVENGNALTSPADR
LKFFQAVAELREPFDRFLADNHPDAVVSDSFFHWSTDAAAEHGVPRLGFL
GSSMFAGSCNESTLHNNPLETAADDPDALVSLPGLPHRVELRRSQMMDPK
KRPDHWALLESVNAADQKSFGEVFNSFHELEPDYVEHYQTTLGRRTWLVG
PVALASKDMAGRGSTSARSPDADSCLRWLDTKQPGSVVYVSFGTLIRFSP
AELHELARGLDLSGKNFVWVLGRAGPDSSEWMPQGFADLITPRGDRGFII
RGWAPQMLILNHRALGGFVTHCGWNSTLESVSAGVPMVTWPRFADQFQNE
KLIVEVLKVGVSIGAKDYGSGIENHDVIRGEVIAESIGKLMGSSEESDAI
QRKAKDLGAEARSAVENGGSSYNDVGRLMDELMARRSSVKVGEDIIPTND
GL
>gi|460409128|ref|XP_004249992.1| PREDICTED:
cyanidin-3-O-glucoside 2-O-glucuronosyl-
transferase-like [Solanum lycopersicum]
SEQ ID 5
MSPKLHKELFFHSLYKKTRSNHTMATLKVLMFPFLAYGHISPYLNVAKKL
ADRGFLIYFCSTPINLKSTIEKIPEKYADSIHLIELHLPELPQLPPHYHT
TNGLPPNLNQVLQKALKMSKPNFSKILQNLKPDLVIYDILQRWAKHVANE
QNIPAVKLLTSGAAVFSYFFNVLKKPGVEFPFPGIYLRKIEQVRLSEMMS
KSDKEKELEDDDDDDDLLVDGNMQIMLMSTSRTIEAKYIDFCTALTNWKV
VPVGPPVQDLITNDVDDMELIDWLGTKDENSTVFVSFGSEYFLSKEDMEE
VAFALELSNVNFIWVARFPKGEERNLEDALPKGFLERIGERGRVLDKFAP
QPRILNHPSTGGFISHCGWNSAMESIDFGVPIIAMPMHLDQPMNARLIVE
LGVAVEIVRDDDGKIHRGEIAETLKGVITGKTGEKLRAKVRDISKNLKTI
RDEEMDAAAEELIQLCRNGN
>gi|222619587|gb|EEE55719.1| hypothetical
protein OsJ_04191 [Oryza sativa Japonica Group]
SEQ ID 6
MHVVMLPWLAFGHILPFAEFAKRVARQGHRVTLFSTPRNTRRLIDVPPSL
AGRIRVVDIPLPRVEHLPEHAEATIDLPSNDLRPYLRRAYDEAFSRELSR
LLQETGPSRPDWVLADYAAYWAPAAASRHGVPCAFLSLFGAAALCFFGPA
ETLQGRGPYAKTEPAHLTAVPEYVPFPTTVAFRGNEARELFKPSLIPDES
GVSESYRFSQSIEGCQLVAVRSNQEFEPEWLELLGELYQKPVIPIGMFPP
PPPQDVAGHEETLRWLDRQEPNSVVYAAFGSEVKLTAEQLQRIALGLEAS
ELPFIWAFRAPPDAGDGDGLPGGFKERVNGRGVVCRGWVPQVKFLAHASV
GGFLTHAGWNSIAEGLANGVRLVLLPLMFEQGLNARQLAEKKVAVEVARD
EDDGSFAANDIVDALRRVMVGEEGDEFGVKVKELAKVFGDDEVNDRYVRD
FLKCLSEYKMQRQG
>gi|297795735|ref|XP_002865752.1| UDP-
glucoronosyl/UDP-glucosyl transferase family
protein [Arabidopsis lyrata subsp. lyrata]
SEQ ID 7
MDDKKEEVMHIAMFPWLAMGHLLPFLRLSKLLAQKGHKISFISTPRNILR
LPKLPSNLSSSITFVSFPLPSISGLPPSSESSMDVPYNKQQSLKAAFDLL
QPPLTEFLRLSSPDWIIYDYASHWLPSIAKELGISKAFFSLFNAATLCFM
GPSSSLIEESRSTPEDFTVVPPWVPFKSTIVFRYHEVSRYVEKTDEDVTG
VSDSVRFGYTIDGSDAVFVRSCPEFEPEWFSLLQDLYRKPVFPIGFLPPV
IEDDDDDTTWVRIKEWLDKQRVNSVVYVSLGTEASLRREELTELALGLEK
SETPFFWVLRNEPQIPDGFEERVKGRGMVHVGWVPQVKILSHESVGGFLT
HCGWNSVVEGIGFGKVPIFLPVLNEQGLNTRLLQGKGLGVEVLRDERDGS
FGSDSVADSVRLVMIDDAGEEIREKVKLMKGLFGNMDENIRYVDELVGFM
RNDESSQLKEEEEEDDCSDDQSSEVSSETDEKELNLDLKEEKRRISVYKS
LSSEFDDYVANEKMG

The tested plasmids were received in a microtiterplate containing a plasmid as freeze-dried solid in each separate well.

Suspension of plasmids. To each well was added 24 μL of ultra-pure sterile water and the microtiter plate was shaken for 30 minutes at Room Temperature. Subsequently, the plate was incubated at 4° C. for 1 hour. The content of each well were further mixed by pipetting up and down. The plasmid quantification was performed by Qubit2.0 analysis using 1 μL of suspension. Determined quantities of plasmids were:

| Microtiter plate | Position | Internal reference | [Plasmid] ng/μL |
|---|---|---|---|
| C908201 | A1 | S115N01 A1 | 32.8 |
| C908201 | G2 | S115N01 G2 | 41.0 |
| C908201 | A7 | S115N05 A7 | 56.6 |
| C912666 | E1 | S115N06 E1 | 64.0 |
| C912666 | C2 | S115N06 C2 | 31.4 |

Transformation of competent cells with plasmids. Aliquots of chemically competent EC100 cells were taken from freezer at −80° C. and stored on ice. The cells were allowed to thaw on ice for 10 minutes. 10 μL of a dilution of above described plasmid solution was added to a sterile microtube of 1.5 mL (in order to transform each cell with 50 pg of DNA) and stored on ice. 100 μL of chemically competent cells was added to each microtube. After incubation of the chemically competent cells plasmid mixtures on ice for 20 min a thermal shock of 30 seconds at 42° C. was performed.

Further incubation was performed on ice for 2 minutes. To each microtube 300 μL of SOC medium was added and the resulting mixture was transferred to a sterile 15 mL tube. After incubate for 1 hour at 37° C. while shaking at 135 rpm, the mixture is spread on solid Luria Broth medium containing Kanamycin 50 pg/mL. The petri-dishes are allowed to incubate for 16 hours at 37° C.

Preparation of stock solutions in glycerol and purification of plasmids. To a 50 mL sterile Falcon Tube 10 mL of Luria Broth medium containing 50 pg/mL of Kanamycin was added. The medium was seeded with an isolated colony from the above described Petri dish and the cultures were allowed to incubate for 16 hours at 37° C. while shaking at 135 rpm.

To sterile microtube of 1.5 mL containing 300 μL of a 60% sterile glycerol solution, 600 μL of the culture was added. The stock solution was stored at −80° C.

The remainder of the culture was centrifuged at 5,525g for 10 minutes at 10° C. and after removal of the supernatant, the pellet was stored on ice. The produced plasmids were purified according to the Qiagen Qiaprep Spin Miniprep kit (ref: 27106) and the plasmid yield was measured at 260 nm. The plasmid solution was stored at 4° C. Plasmid quantities were determined as follows:

| Microtiter plate | Position | Internal reference of test | [Plasmid] ng/μL |
|---|---|---|---|
| C908201 | A1 | S115N01 A1 | 115.7 |
| C908201 | G2 | S115N01 G2 | 120.4 |
| C908201 | A7 | S115N05 A7 | 293.8 |
| C912666 | E1 | S115N06 E1 | 126.1 |
| C912666 | C2 | S115N06 C2 | 98.8 |

In-vitro Expression of enzymes. 18 μL of plasmid solution (containing approximately 1.5 pg of plasmid) was used for in-vitro expression according to the Promega S30 T7 High-Yield Protein Expression System (ref:L1110) kit. The expression medium was produced as follows:

| | S30 Premix Plus | T7 S30 Extract | Total |
|---|---|---|---|
| Trials | 30 μL | 27 μL | 57 μL |
| reference | 20 μL | 18 μL | 38 μL |

The prepared expression medium mix was added to the plasmid solution and the solution was allowed to incubate at 30° C. for 3 hours while mixing the mixture every 45 minutes. 5 μL of the mixture was frozen whereas the remainder was used for the catalytic test for the conversion of Rebaudioside A to Rebaudioside D.

Catalytic test for transformation of Rebaudioside A to Rebaudioside D. 430 μL of a reaction mixture containing 0.5 mM Rebaudioside A, 3 mM MgCl₂, 50 mM phosphate buffer (pH7.2) and 2.5 mM UDP-glucose was added to a 1.5 mL sterile microtube. 52 μL of the enzyme expression medium was added and the resulting mixture was allowed to react at 30° C. for 24 hours. 125 μL samples were taken after 2 hours, 16 hours and 24 hours and added to a 115 μL of 60% methanol and 10 μL of 2 N H₂SO₄. The quenched sample was centrifuged at 18,000 g for 2 minutes at RT. 200 μL was transferred to an HPLC vial and analyzed.

HPLC Analysis The HPLC assay was performed as follows:

### Apparatus

| Equipment | Supplier | Reference | Lot# |
|---|---|---|---|
| Elite | Hitachi | L-2130 | NA |
| Photodiode Array | Hitachi | L-2455 | NA |
| Corona CAD detector | ESA | 70-6186A | CO-2044 |
| Injector 100 μL | Hitachi | | NA |
| Column Synergy 4u | Phenomenex | 00G-4375-E0 | 588582-12 |
| Hydro-RP 80A (250 × | | | |
| 4.60 mm) | | | |

### Instrument Conditions

| Column Temperature | 55° | C. |
|---|---|---|
| Detection | UV 205 nm; bw 400 nm | |
| | CAD detection | |
| Analysis duration | 15 | min |
| Injected volume | 10 | μL |
| Flow rate | 1 | mL/min |

### Mobile Phase Gradient Program

| | % Water containing | |
|---|---|---|
| Time (min) | 0.04% acetic acid | % methanol |
| 0 | 40 | 60 |
| 8 | 25 | 75 |
| 10 | 25 | 75 |
| 11 | 40 | 60 |
| 15 | 40 | 60 |

The HPLC assay results are provided below:

| Internal | Steviol glycoside conversion in reaction mixture (% area) | | |
|---|---|---|---|
| reference | Reb D | Reb UNK | Reb A |
| S115N01 A1 | 2.1 | ND | 96.7 |
| S115N01 G2 | 0.6 | ND | 99.4 |
| S115N05 A7 | 22.4 | 23.3 | 46.7 |
| S115N06 E1 | 0.14 | 7.0 | 92.8 |
| S115N06 C2 | 0.28 | 3.9 | 95.8 |

The enzyme S 115N05 A7 had the highest activity for Reb A to Reb D conversion (ca. 22.4%)

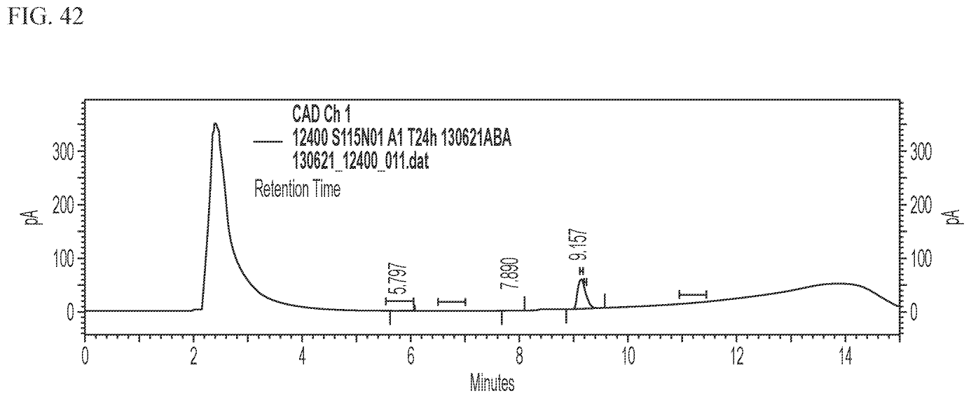

At least three enzymes produced a significant amount of an unknown glycoside (marked as Reb UNK; later identified as reb D2) along with reb D, as seen in FIGS. 42-46.

The below table accompanies FIG. 42.

Sample: 12400 S115N01A1 T24 h 130621ABA
gi|115454819|ref|NP_001051010.1| Os03g0702500
[Oryza sativa Japonica Group]
Filename: 130621_12400_011.dat
CAD Ch 1 Results

| Compound | Retention time | Integration (area) |
|---|---|---|
| Rebaudioside D | 5.797 | 13,532,277 |
| Unknown@RT7.890 | 7.890 | 7,094,778 |
| Rebaudioside A | 9.157 | 613,483,011 |
| Total | | 634,110,066 |

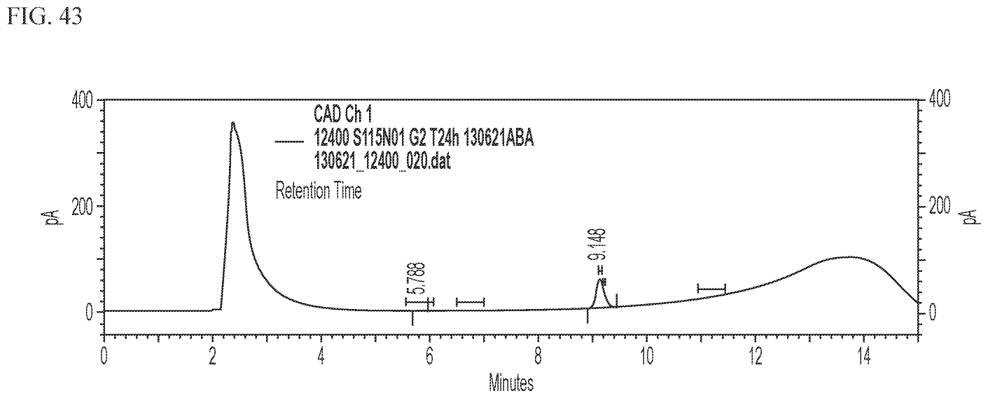

The below table accompanies FIG. 43.

Sample: 12400 S115N01G2 T24 h 130621ABA
>gi|187373030|gb|ACD03249.1|
UDP-glycosyltransferase [Avena strigosa]
Filename: 130621_12400_020.dat
CAD Ch 1 Results

| Compound | Retention time | Integration (area) |
|---|---|---|
| Rebaudioside D | 5.788 | 3,547,834 |
| Rebaudioside A | 9.148 | 585,285,463 |
| Total | | 588,833,297 |

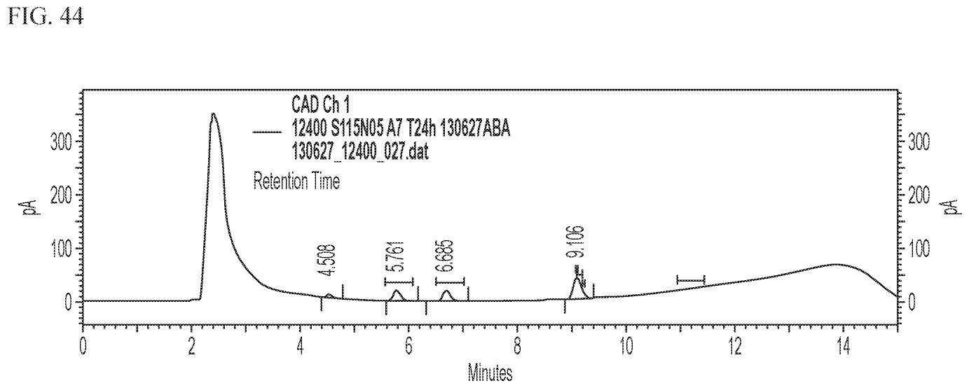

The below table accompanies FIG. 44.PG 49T

Sample: 12400 S115N05A7 T24 h 130627ABA
>gi|460409128|ref|XP_004249992.1| PREDICTED:
cyanidin-3-O-glucoside 2-O-glucuronosyltransferase-like
[Solanum lycopersicum]
Filename: 130627_12400_027.dat
CAD Ch 1 Results

| Compound | Retention time | Integration (area) |
|---|---|---|
| Unknown@RT4.508 | 4.508 | 64,361,822 |
| Rebaudioside D | 5.761 | 191,273,935 |
| Rebaudioside UNK | 6.685 | 198,934,644 |
| Rebausioside A | 9.106 | 398,115,681 |
| Total | | 852,686,082 |

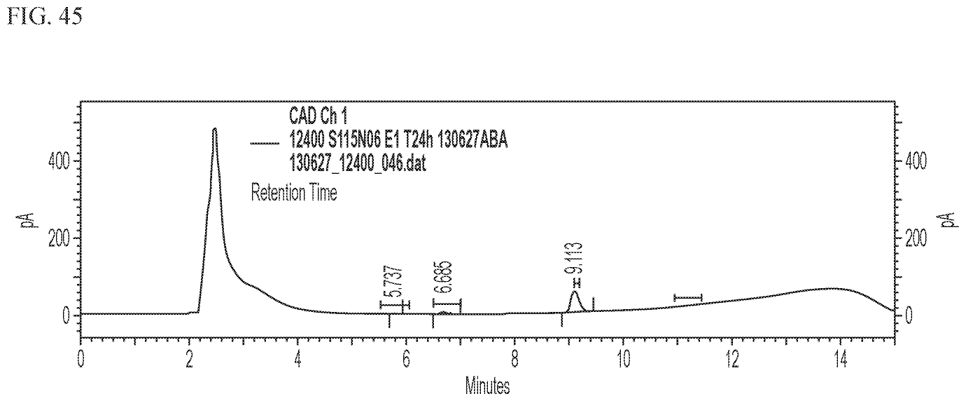

The below table accompanies FIG. 45.

Sample: 12400 S115N06E1 T24 h 130627ABA
>gi|222619587|gb|EEE55719.1| hypothetical protein
OsJ_04191 [Oryza sativa Japonica Group]
Filename: 130627_12400_046.dat
CAD Ch 1 Results

| Compound | Retention time | Integration (area) |
|---|---|---|
| Rebaudioside D | 5.737 | 964,715 |
| Rebaudioside UNK | 6.685 | 46,027,361 |
| Rebausioside A | 9.113 | 606,312,523 |
| Total | | 653,304,599 |

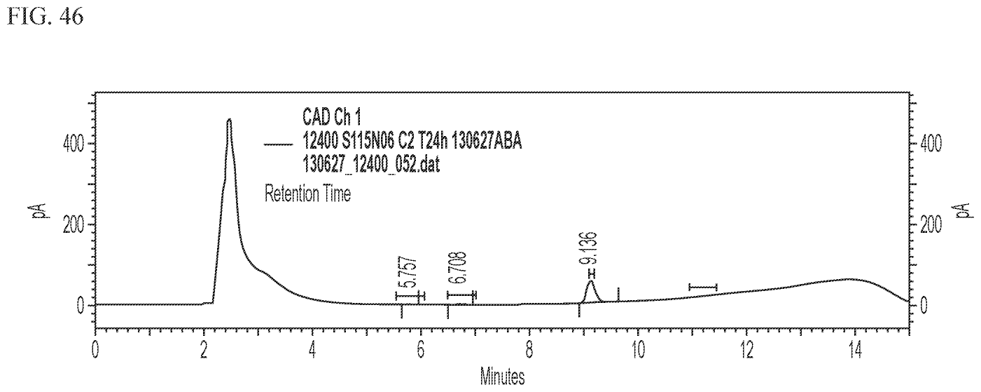

The below table accompanies FIG. 46.

Sample: 12400 S115N06C2 T24 h 130627ABA
>gi|297795735|ref|XP_002865752.1| UDP-glucoronosyl/UDP-glucosyl
transferase family protein [Arabidopsis lyrata subsp. lyrata]
Filename: 130627_12400_052.dat
CAD Ch 1 Results

| Compound | Retention time | Integration (area) |
|---|---|---|
| Rebaudioside D | 5.757 | 1,852,407 |
| Rebaudioside UNK | 6.708 | 26,033,636 |
| Rebausioside A | 9.136 | 633,014,654 |
| Total | | 660,900,697 |

### Example 21

### Activity of In-Vitro Produced EUGT11

EUGT11 gene as was described in the Patent application WO/2013/022989A2 was synthesized by DNA2.0 and subsequently subcloned in pET30a+ vector.

| | | | | | Conversion |
|---|---|---|---|---|---|
| Micro- | Posi- | GI | | Internal | RebA to |
| plate | tion | number | Version | reference | RebD |
| C912666 | G4 | 41469452 | AAS07253.1 | S115N08 G4 | Active |

The amino-acid sequence is as follows:

>gi|41469452|gb|AAS07253.1| putative UDP-
glucoronosyl and UDP-glucosyl transferase
[Oryza sativa Japonica Group] EUGT11 enzyme
from patent application WO/2013/022989A2
SEQ ID 8
MHVVICPLLAFGHLLPCLDLAQRLACGHRVSFVSTPRNISRLPPVRPSLA
PLVSFVALPLPRVEGLPNGAESTHNVPHDRPDMVELHLRAFDGLAAPFSE
FLGTACADWVMPTSSAPRQTLSSNIHRNSSRPGTPAPSGRLLCPITPHSN
TLERAAEKLVRSSRQNARARSLLAFTSPPLPYRDVFRSLLGLQMGRKQLN
IAHETNGRRTGTLPLNLCRWMWKQRRCGKLRPSDVEFNTSRSNEAISPIG
ASLVNLQSIQSPNPRAVLPIASSGVRAVFIGRARTSTPTPPHAKPARSAA
PRAHRPPSSVMDSGYSSSYAAAAGMHVVICPWLAFGHLLPCLDLAQRLAS
RGHRVSFVSTPRNISRLPPVRPALAPLVAFVALPLPRVEGLPDGAESTND
VPHDRPDMVELHRRAFDGLAAPFSEFLGTACADWVIVDVFHHWAAAAALE
HKVPCAMMLLGSAHMIASIADRRLERAETESPAAAGQGRPAAAPTFEVAR
MKLIRTKGSSGMSLAERFSLTLSRSSLVVGRSCVEFEPETVPLLSTLRGK
PITFLGLMPPLHEGRREDGEDATVRWLDAQPAKSVVYVALGSEVPLGVEK
VHELALGLELAGTRFLWALRKPTGVSDADLLPAGFEERTRGRGVVATRWV
PQMSILAHAAVGAFLTHCGWNSTIEGLMFGHPLIMLPIFGDQGPNARLIE
AKNAGLQVARNDGDGSFDREGVAAAIRAVAVEEESSKVFQAKAKKLQEIV
ADMACHERYIDGFIQQLRSYKD

The tested plasmid was received in a microtiterplate containing a plasmid as freeze-dried solid in a separate well.

Suspension of plasmid To the well was added 24 μL of ultra-pure sterile water and the microtiter plate was shaken for 30 minutes at Room Temperature. Subsequently, the plate was incubated at 4° C. for 1 hour. The content of the well was further mixed by pipetting up and down. The plasmid quantification was performed by Qubit2.0 analysis using 1 μL of suspension. Plasmid quantity was determined as follows:

| | | Internal reference | |
|---|---|---|---|
| Microtiter plate | Position | of test | [Plasmid] ng/μL |
| C912666 | G4 | S115N08 G4 | 19.2 |

Transformation of competent cells with plasmid. An aliquot of chemically competent EC100 cells was taken from freezer at −80° C. and stored on ice. The cells were allowed to thaw on ice for 10 minutes. 10 μL of a dilution of above described plasmid solution was added to a sterile microtube of 1.5 mL (in order to transform each cell with 50 pg of DNA) and stored on ice. 100 μL of chemically competent cells was added to the microtube. After incubation of the chemically competent cells/plasmid mixture on ice for 20 min a thermal shock of 30 seconds at 42° C. was performed.

Further incubation was performed on ice for 2 minutes. To the microtube 300 μL of SOC medium was added and the resulting mixture was transferred to a sterile 15 mL tube. After incubate for 1 hour at 37° C. while shaking at 135 rpm, the mixture is spread on solid Luria Broth medium containing Kanamycin 50 pg/mL. The Petri dish is allowed to incubate for 16 hours at 37° C.

Preparation of stock solutions in glycerol and purification of plasmid. To a 50 mL sterile Falcon Tube 10 mL of Luria Broth medium containing 50 pg/mL of Kanamycin was added. The medium was seeded with an isolated colony from the above described Petri dish and the cultures were allowed to incubate for 16 hours at 37° C. while shaking at 135 rpm.

To sterile microtube of 1.5 mL containing 300 μL of a 60% sterile glycerol solution, 600 μL of the culture was added. The stock solution was stored at −80° C.

The remainder of the culture was centrifuged at 5,525 g for 10 minutes at 10° C. and after removal of the supernatant, the pellet was stored on ice. The produced plasmids were purified according to the Qiagen Qiaprep Spin Miniprep kit (ref: 27106) and the plasmid yield was measured at 260 nm. The plasmid solution was stored at 4° C. Plasmid quantity was determined as follows:

| | | Internal reference | |
|---|---|---|---|
| Microtiter plate | Position | of test | [Plasmid] ng/μL |
| C912666 | G4 | S115N08 G4 | 38.4 |

In-vitro Expression of EUGT11. 18 μL of a diluted plasmid solution (containing approximately 1.5 pg of plasmid) was used for in-vitro expression according to the Promega S30 T7 High-Yield Protein Expression System (ref:L1110) kit. The expression medium was produced as follows:

| | S30 Premix Plus | T7 S30 Extract | DNA template | Total |
|---|---|---|---|---|
| Trials | 30 μL | 27 μL | 18 μL (~1.5 μg) | 75 μL |
| reference | 20 μL | 18 μL | 12 μL (~1.0 μg) | 50 μL |

The prepared expression medium mix was added to the plasmid solution and the solution was allowed to incubate at 30° C. for 3 hours while mixing the mixture every 45 minutes. 5 μL of the mixture was frozen whereas the remainder was used for the catalytic test for the conversion of Rebaudioside A to Rebaudioside D.

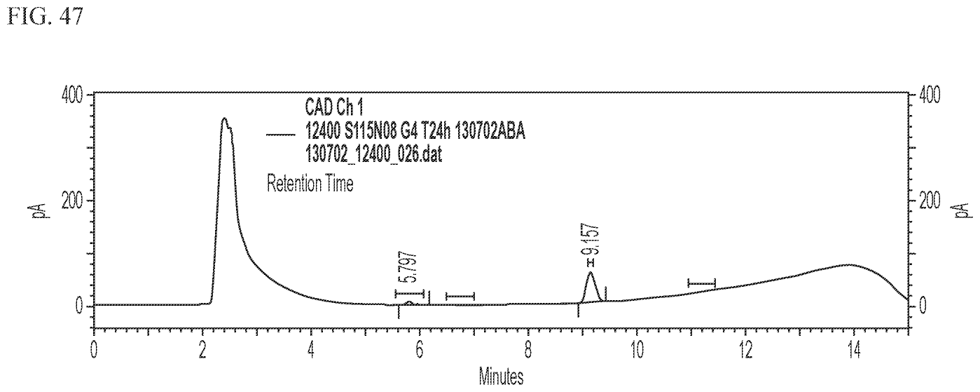

Catalytic test for transformation of Rebaudioside A to Rebaudioside D. 430 μL of a reaction mixture containing 0.5 mM Rebaudioside A, 3 mM MgCl₂, 50 mM phosphate buffer (pH7.2) and 2.5 mM UDP-glucose was added to a 1.5 mL sterile microtube. 52 μL of the enzyme expression medium was added and the resulting mixture was allowed to react at 30° C. for 24 hours. 125 μL samples were taken after 2 hours, 16 hours and 24 hours and added to a 115 μL of 60% methanol and 10 μL of 2 N H₂SO₄. The quenched sample was centrifuged at 18,000 g for 2 minutes at RT. 200 μL was transferred to HPLC vial and analyzed as seen in FIG. 47.

HPLC Analysis. The HPLC assay was performed as described in EXAMPLE 20.

The HPLC assay results are provided below:

Sample: 12400 S115N08G4 T24 h 130702CJA
>gi|41469452|gb|AAS07253.1| putative UDP-glucoronosyl and
UDP-glucosyl transferase [Oryza sativa Japonica Group]
(EUGT11 enzyme from Patent application WO/2013/022989A2)
Filename: 130702_12400_026.dat
CAD Ch 1 Results

| Compound | Retention time | Integration (area) |
|---|---|---|
| Rebaudioside D | 5.797 | 54,654,810 |
| Rebaudioside A | 9.157 | 633,926,835 |
| Total | | 688,581,645 |

### Example 22

In-vivo production of enzymes

The enzymes described in EXAMPLE 20 were produced in vivo.

The pET30A+ vector containing the gene corresponding to the enzyme was introduced in E. coli BL21(DE3) by heat shock. The obtained cells were grown in Petri dishes in the presence of Kanamycin and suitable colonies were selected and allowed to grow in liquid LB medium (Erlenmeyer flasks). Glycerol was added to the suspension as cryoprotector and 400 μL aliquots were stored at −20° C. and at −80° C.

The storage aliquots of E. coli BL21(DE3) containing the pET30A+_UGT plasmids were thawed and added to 30 mL of LBGKP medium (20 g/L Luria Broth Lennox; 50 mM PIPES buffer pH 7.00; 50 mM Phosphate buffer pH 7.00; 2.5 g/L glucose and 50 mg/L of Kanamycine). This culture was allowed to shake at 135 rpm at 30° C. for 8 hrs.

The production medium contained 60 g/L of overnight express instant TB medium (Novagen), 10 g/L of glycerol and 50 mg/L of Kanamycine. The preculture was added to 400 mL of this medium and the solution was allowed to stir at 20° C. while taking samples to measure the OD and pH. The cultures gave significant growth and a good OD was obtained. After 40 hrs, the cells were harvested by centrifugation and frozen. The following yields of cell wet weights (CWW) are mentioned below.

| GI number | Version | CWW |
|---|---|---|
| 115454819 | NP_001051010.1 | 9.2 g |
| 187373030 | ACD03249.1 | 7.4 g |
| 460409128 | XP_004249992.1 | 6.8 g |
| 222619587 | EEE55719.1 | 7.5 g |
| 297795735 | XP_002865752.1 | 8.8 g |

Lysis was performed by addition of Bugbuster Master mix (Novagen) and the lysate was recovered by centrifugation and used fresh.

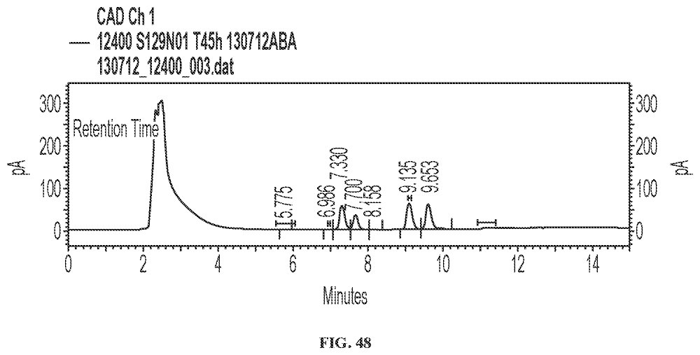

Determination of activity. Activity tests were performed at 5 mL scale with 1,000 μL of thawed lysate for the transformation of Rebaudioside A using 0.5 mM of substrate, 2.5 mM of UDP-Glucose and 3 mM MgCl₂ in 50 mM Sodium Phosphate buffer at pH 7.2. Samples were taken and analyzed by HPLC as seen in FIGS. 48-52.

HPLC Analysis. The HPLC assay was performed as described in EXAMPLE 20.

The results for the different enzymes are provided below.

| | | Conversion | |
|---|---|---|---|
| GI Number | Version | after 45 hrs. | Reb D selectivity |
| 115454819 | NP_001051010.1 | 1.1% | 100% |
| 187373030 | ACD03249.1 | 0.8% | 100% |
| 460409128 | XP_004249992.1 | 62.1% | 43.6% |
| 222619587 | EEE55719.1 | 2.9% | Reb D Not detected |
| 297795735 | XP_002865752.1 | 0.0% | Reb D Not detected |

The below table accompanies FIG. 48.

SAMPLE: 12400 S129N01 T45 h 130712ABA
>gi115454819/NP_001051010.1
Filename: 130712_12400_003.dat
CAD Ch 1 Results

| Compound | Retention time | Integration (area) |
|---|---|---|
| Rebaudioside D | 5.775 | 3,264,475 |
| Unknown@RT6.986 | 6.986 | 4,110,607 |
| Unknown@RT7.330 | 7.330 | 564,033,104 |
| Unknown@RT7.700 | 7.700 | 328,710,539 |
| Unknown@RT8.158 | 8.158 | 6,344,796 |
| Rebaudioside A | 9.135 | 673,271,863 |
| Unknown@RT9.653 | 9.653 | 616,489,141 |
| Total | | 2,196,224,525 |

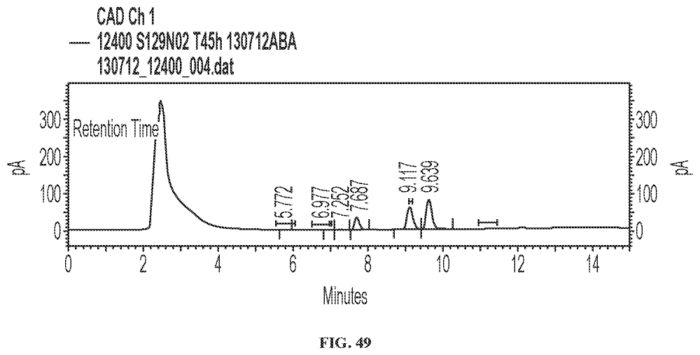

The below table accompanies FIG. 49.

Sample: 12400 S129N02 T45 h 130712ABA
>gi187373030/ACD03249.1
Filename: 130712_12400_004.dat
CAD Ch 1 Results

| Compound | Retention time | Integration (area) |
|---|---|---|
| Rebaudioside D | 5.772 | 1,997,401 |
| Unknown@RT6.977 | 6.977 | 3,341,419 |
| Unknown@RT7.252 | 7.252 | 10,576,676 |
| Unknown@RT7.687 | 7.687 | 298,862,034 |
| Rebaudioside A | 9.117 | 675,210,845 |
| Unknown@RT9.639 | 9.639 | 874,680,345 |
| Total | | 1,864,668,720 |

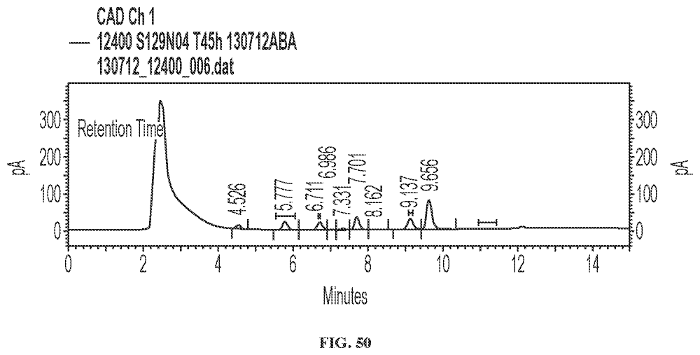

The below table accompanies FIG. 50.

Sample: 12400 S129N04 T45 h 130712ABA
>gi460409128/XP_004249992.1
Filename: 130712_12400_006.dat
CAD Ch 1 Results

| Compound | Retention time | Integration (area) |
|---|---|---|
| Unknown@RT4.526 | 4.526 | 89,809,997 |
| Rebaudioside D | 5.777 | 217,830,913 |
| Rebaudioside UNK | 6.711 | 192,129,243 |
| Unknown@RT6.986 | 6.986 | 10,241,417 |
| Unknown@RT7.331 | 7.331 | 41,195,765 |
| Unknown@RT7.701 | 7.701 | 310,640,254 |
| Unknown@RT8.162 | 8.162 | 7,950,609 |
| Rebaudioside A | 9.137 | 304,611,760 |
| Unknown@RT9.656 | 9.656 | 863,496,704 |
| Total | | 2,037,906,662 |

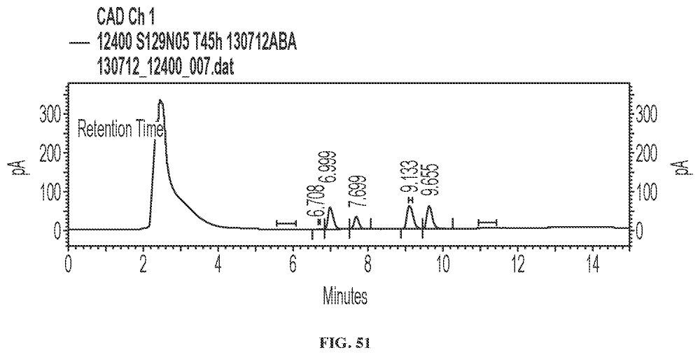

The below table accompanies FIG. 51.

Sample: 12400 S129N05 T45 h 130712ABA
>gi222619587/EEE55719.1
Filename: 130712_12400_007.dat
CAD Ch 1 Results

| Compound | Retention time | Integration (area) |
|---|---|---|
| Rebaudioside UNK | 6.708 | 20,047,847 |
| Unknown@RT6.999 | 6.999 | 598,924,958 |
| Unknown@RT7.699 | 7.699 | 303,182,042 |
| Rebaudioside A | 9.133 | 672,777,773 |
| Unknown@RT9.655 | 9.655 | 606,371,969 |
| Total | | 2,201,304,589 |

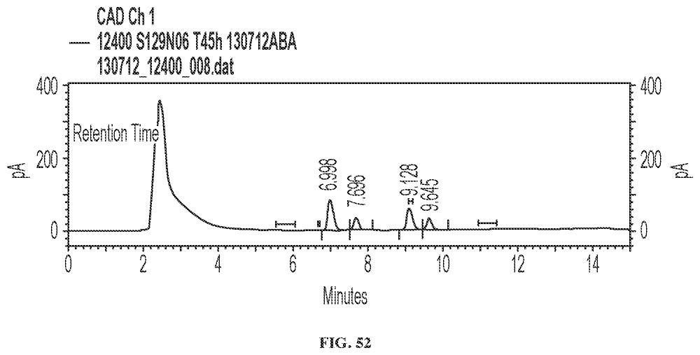

The below table accompanies FIG. 52.

Sample: 12400 S129N06 T45 h 130712ABA
>gi297795735/XP_002865752.1
Filename: 130712_12400_008.dat
CAD Ch 1 Results

| Compound | Retention time | Integration (area) |
|---|---|---|
| Unknown@RT6.998 | 6.998 | 920,620,332 |
| Unknown@RT7.696 | 7.696 | 314,421,575 |
| Rebaudioside A | 9.128 | 688,195,594 |
| Unknown@RT9.645 | 9.645 | 308,115,680 |
| Total | | 2,231,353,181 |

### Example 23

### Identification of Glycosides

The reaction mixtures representing GI No. 460409128, particularly the sample “12400 S115N05A7 T24h 130627ABA” of EXAMPLE 20 (hereinafter S115N05A7), and the sample “12400 S129N04 T45h 130712ABA” of EXAMPLE 22 (hereinafter S129N04) were additionally assayed by LC-MS to identify the unknown glycosides. An Agilent 1200 series HPLC system, equipped with binary pump (G1312B), autosampler (G1367D), thermostated column compartment (G1316B), DAD detector (G1315C), connected with Agilent 6110A MSD, and interfaced with “LC/MSD Chemstation” software, was used.

### Instrument Conditions

| Column | Phenomenex Kinetex 2.6 u C18 100 A, | |
|---|---|---|
| | 4.6 mm × 150 mm, 2.6 μm | |
| Column Temperature | 55° | C. |
| Detection | DAD at 210 nm bw 360 nm | |
| | MSD (Scan and SIM mode) | |
| | Mode: ES-API, Negative Polarity | |
| | Drying gas flow: 13.0 L/min | |
| | Nebulizer pressure: 30 psig | |
| | Drying gas temperature: 270° C. | |
| Analysis duration | 25 | min |
| Injected volume | 2 | μL |
| Flow rate | 1 | mL/min |

### Mobile Phase Gradient Program

| Time (min) | A (%): Formic acid 0.1% | B (%): Acetonitrile |
|---|---|---|
| 0 | 75 | 25 |
| 8.5 | 75 | 25 |
| 10.0 | 71 | 29 |
| 16.5 | 70 | 30 |

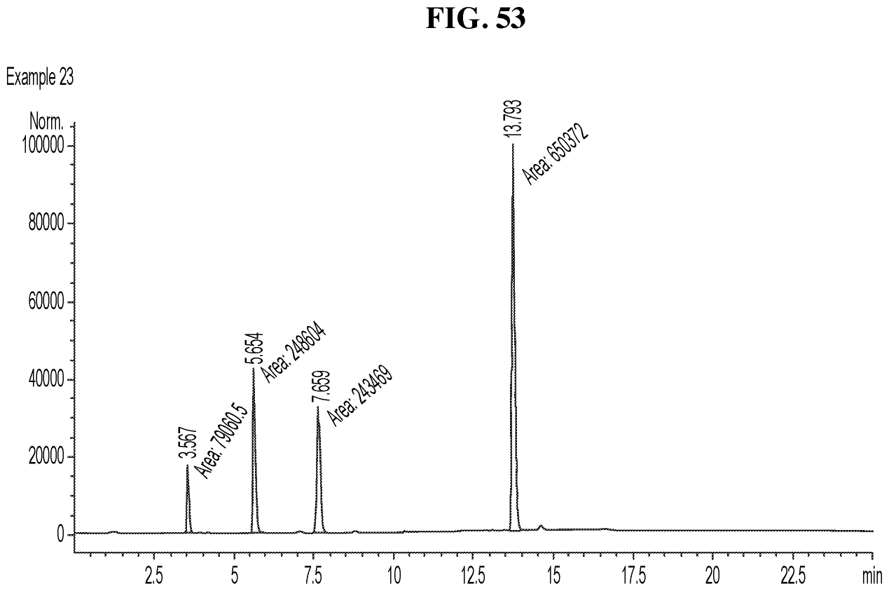

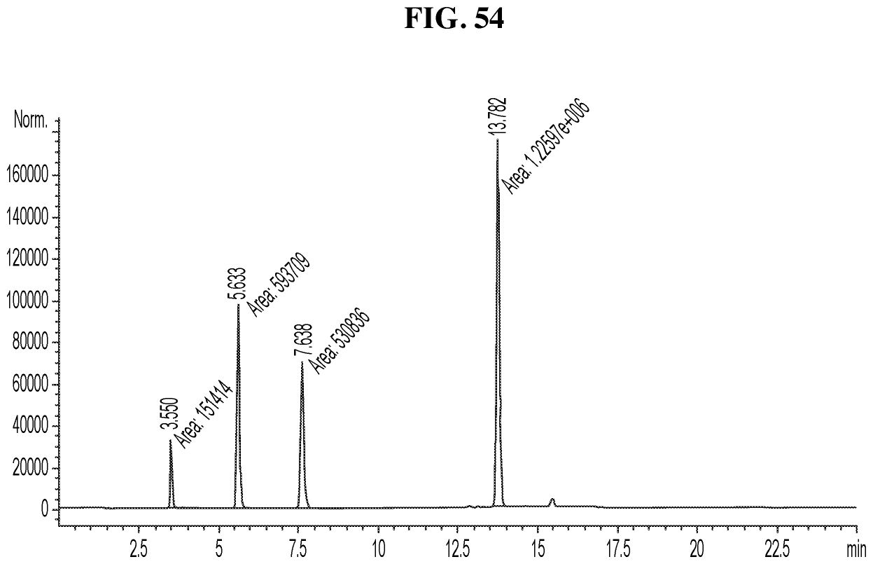

The compound observed on LCMS system at 3.5 min, corresponds to compound “Unknown@4.508” in sample “S115N05A7” (EXAMPLE 20), and compound “Unknown@RT4.526” in sample “S129N04” (EXAMPLE 22). The LCMS data suggests that this compound has six glucosidic residues (C56H₉₀O₃₃) in its structure, and was found to be an isomer form of reb M, namely reb M2 (see Example 40 for discussion).

Whereas the compound observed on LCMS system at 7.6 min, corresponds with compound “reb UNK” in sample “S115N05A7” (EXAMPLE 20), and compound “reb UNK” in sample “S129N04” (EXAMPLE 22), The LCMS data suggests that “reb UNK” has five glucosidic residues (C₅₀H₈₀O₂₈) in its structure, and was found to be an isomer form of reb D, namely reb D2 (see Example 39 for discussion). The ratio of these compounds and the LCMS chromatograms are provided below and as shown in FIGS. 53-54.

| | Steviol glycoside conversion in reaction mixture (% area) | | | |
|---|---|---|---|---|
| Sample | Unknown@RT3.5 | Reb D | Reb UNK | Reb A |
| S115N05A7 | 6.47 | 20.35 | 19.93 | 53.24 |
| S129N04 | 6.05 | 23.73 | 21.22 | 49.00 |

The below table accompanies FIG. 53.

Sample: 12400 S115N05A7 T24 h 130627ABA
>gi|460409128/XP_004249992.1
MSD SIM Results

| Compound | Retention time | MW | Integration (area) |
|---|---|---|---|
| Unknown@RT3.567 | 3.567 | 1,291 | 79,060 |
| Rebaudioside D | 5.654 | 1,129 | 248,604 |
| Rebaudioside UNK | 7.659 | 1,129 | 243,469 |
| Rebausioside A | 13.793 | 967 | 650,372 |
| Total | | | 1,221,505 |

The below table accompanies FIG. 54.

Sample: 12400 S129N04 T45 h 130712ABA
>gi460409128/XP_004249992.1
MSD SIM Results

| Compound | Retention time | MW | Integration (area) |
|---|---|---|---|
| Unknown@RT3.550 | 3.550 | 1,291 | 151,414 |
| Rebaudioside D | 5.633 | 1,129 | 593,709 |
| Rebaudioside UNK | 7.638 | 1,129 | 530,836 |
| Rebaudioside A | 13.782 | 967 | 1,225,970 |
| Total | | | 2,501,929 |

### Example 24

### Identification of Glycosides

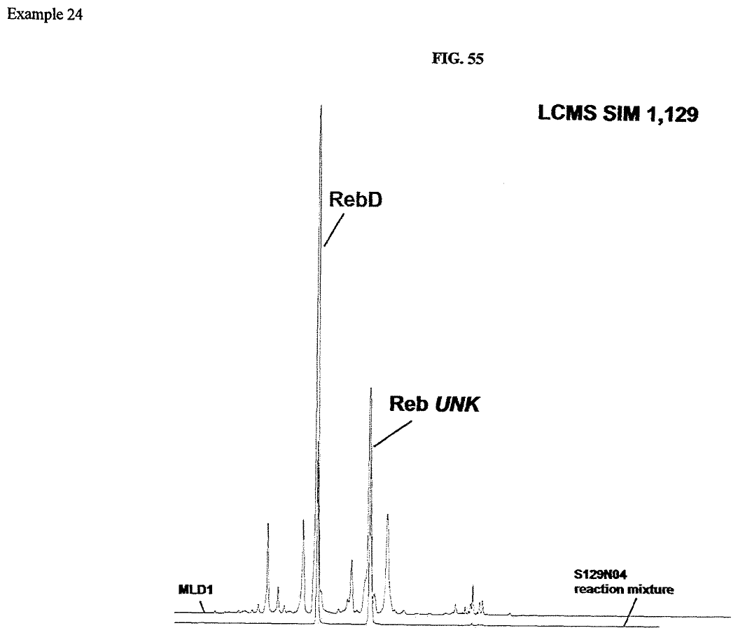

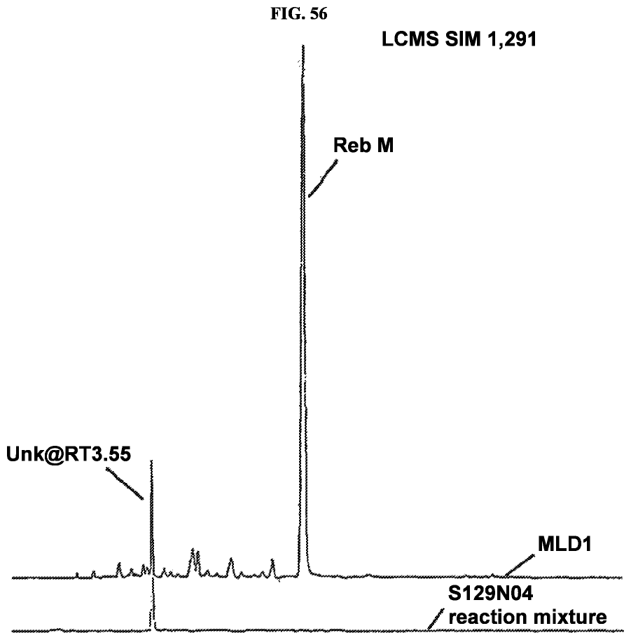

The reaction mixture representing GI No. 460409128, particularly the sample “12400 S129N04 T45h 130712ABA” of EXAMPLE 22 (hereinafter S129N04) were additionally assayed by LC-MS, as seen in FIGS. 55-56, along with Stevia rebaudiana Bertoni leaf extract “MLD1” produced by PureCircle Sdn Bhd (Malaysia) to determine the occurrence of S129N04 glycosides in nature.

The below table accompanies FIG. 55.

Samples:
1) 12400 S129N04 T45 h 130712ABA
>gi460409128/XP_004249992.1
2) MLD1 Stevia rebaudiana Bertoni extract
MSD SIM 1,129 Results

| Compound | MW |
|---|---|
| Rebaudioside D | 1,129 |
| Rebaudioside UNK | 1,129 |

The below table accompanies FIG. 56.

Samples:
1) 12400 S129N04 T45 h 130712ABA
>gi460409128/XP_004249992.1
2) MLD1 Stevia rebaudiana Bertoni extract
MSD SIM 1,291 Results

| Compound | MW |
|---|---|
| Unknown@RT3.550 | 1,291 |
| Rebaudioside M | 1,291 |

The assay shows that the compound observed on LCMS system at 3.5 min, in EXAMPLE 23 (C₅₆H₉₀O₃₃; later confirmed as reb M2), and the compound observed on LCMS system at 7.6 min, in EXAMPLE 23 (C₅₀H₈₀O₂₈; reb UNK, later confirmed as reb D2) occur in the extract of Stevia rebaudiana Bertoni plant.

### Example 25

### Conversion of Rebaudioside E to Rebaudioside D

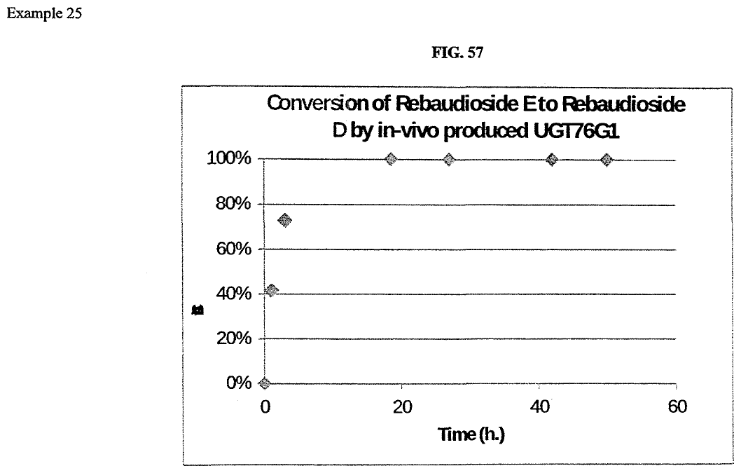

The total volume of the reaction was 5.0 mL with the following composition: 100 mM potassium phosphate buffer pH 7.5, 3 mM MgCl₂, 2.5 mM UDP-glucose, 0.5 mM Rebaudioside E and 500 μL of UGT76G1 thawed lysate (UGT76G1 gene was cloned in pET30a+ vector and expressed in E. coli BL21 (DE3)). The reactions were run at 30° C. on an orbitary shaker at 135 rpm. For sampling 300 μL of the reaction mixture was quenched with 30 μL of 2N H₂SO₄ and 270 μL of methanol/water (6/4). The samples were immediately centrifuged and kept at 10° C. before analysis by HPLC (CAD detection). The following reaction profile was obtained corresponding to a complete conversion of Rebaudioside E to Rebaudioside D as seen in FIG. 57.

### Example 26

### Directed Evolution of UGT76G1 for the Conversion of Rebaudioside D to Rebaudioside M

Starting from the amino acid sequence of UGT76G1, as is described in Genbank (AAR06912.1), different mutations at various amino acid positions were identified that could alter the activity of the enzyme for the transformation of Rebaudioside D (Reb D) to Rebaudioside M (Reb M). This list of mutations, designed by DNA2.0 ProteinGPS™ strategy, was subsequently used to synthesize 96 variant genes that contained 3, 4 or 5 of these mutations that were codon-optimized for expression in E. coli. The genes were subcloned in the pET30a+ plasmid and used for transformation of E. coli BL21 (DE3) chemically competent cells. The obtained cells were grown in Petri-dishes on solid LB medium in the presence of Kanamycin. Suitable colonies were selected and allowed to grow in liquid LB medium in tubes. Glycerol was added to the suspension as cryoprotectant and 400 μL aliquots were stored at −20° C. and at −80° C.

These storage aliquots of E. coli BL21(DE3) containing the pET30a+_UGT76Glvar plasmids were thawed and added to LBGKP medium (20 g/L Luria Broth Lennox; 50 mM PIPES buffer pH 7.00; 50 mM Phosphate buffer pH 7.00; 2.5 g/L glucose and 50 mg/L of Kanamycine). This culture was allowed to shake in a 96 microtiter plate at 135 rpm at 30° C. for 8 h. 3.95 mL of production medium containing 60 g/L of Overnight Express™ Instant TB medium (Novagen®), 10 g/L of glycerol and 50 mg/L of Kanamycin was inoculated with 50 μL of above described culture. In a 48 deepwell plate the resulting culture was allowed to stir at 20° C. The cultures gave significant growth and a good OD (600 nm; 1 cm) was obtained. After 44 h, the cells were harvested by centrifugation and frozen.

Lysis was performed by addition of Bugbuster® Master mix (Novagen®) to the thawed cells and the lysate was recovered by centrifugation. Activity tests were performed with 100 μL of fresh lysate that was added to a solution of Rebaudioside D (final concentration 0.5 mM), MgCl₂ (final concentration 3 mM) and UDP-Glucose (final concentration 2.5 mM) in 50 mM phosphate buffer pH 7.2.

The reaction was allowed to run at 30° C. and samples were taken after 2, 4, 7 and 24 h. to determine conversion and initial rate by HPLC (CAD detection) using the analytical method that was described above for the transformation of Rebaudioside D to Rebaudioside M. The results are depicted in the following table.

| | | conversion Reb D | initial rate |
|---|---|---|---|
| | | to Reb M after 24 h | (Reb M |
| Clone | Mutations* | (%) | area/min) |
| UGT76G1var1 | E224A_F314S_R334K | 51.8 | 5.5E+07 |
| UGT76G1var2 | S274G_T284I_L379G | 49.3 | 4.7E+07 |
| UGT76G1var3 | I295T_S357C_V366I | 9.6 | 1.6E+06 |
| UGT76G1var4 | E224D_E231A_F265I | 14.7 | 8.6E+06 |
| UGT76G1var5 | F22Y_I373L_P382M | 3.5 | 2.3E+06 |
| UGT76G1var6 | Q266S_S357N_I373L | 0.5 | 1.8E+06 |
| UGT76G1var7 | F22L_I43V_A239V | 0.2 | −6.0E+04 |
| UGT76G1var8 | E224A_Q266S_Q342E | 0.5 | 2.3E+04 |
| UGT76G1var9 | E231A_D301N_G348P | 52.0 | 4.9E+07 |
| UGT76G1var10 | A33G_L246F_Q342E | 0.3 | −7.7E+02 |
| UGT76G1var11 | F22L_A33G_V310I | 0.4 | 3.8E+04 |
| UGT76G1var12 | L243P_K303G_A352G | 0.5 | 8.7E+04 |
| UGT76G1var13 | L243A_S357C_A385T | 0.2 | −3.3E+04 |
| UGT76G1var14 | A239I_F265I_V396F | 5.3 | 1.5E+06 |
| UGT76G1var15 | F41L_L246F_Q425E | 5.6 | 1.5E+06 |
| UGT76G1var16 | F265I_P272A_I335V | 18.6 | 5.8E+06 |
| UGT76G1var17 | F265L_Q266E_Q342K | 0.7 | 7.2E+05 |
| UGT76G1var18 | L243P_S274G_N409R | 1.9 | 5.0E+05 |
| UGT76G1var19 | E224D_E229A_Q432E | 10.5 | 5.5E+06 |
| UGT76G1var20 | S375M_K393G_Y397E | 1.8 | 1.9E+06 |
| UGT76G1var21 | A239V_V300A_K303G | 41.9 | 3.3E+07 |
| UGT76G1var22 | E231A_V310I_R334K | 34.4 | 2.4E+07 |
| UGT76G1var23 | T263S_G348P_A352G | 47.8 | 4.1E+07 |
| UGT76G1var24 | A239I_P272A_Q425E | 31.0 | 2.1E+07 |
| UGT76G1var25 | T284L_Q342K_Y397Q | 0.9 | 6.3E+04 |
| UGT76G1var26 | S241I_F265L_F377C | 1.8 | 7.5E+05 |
| UGT76G1var27 | A239I_L379A_V394I | 29.0 | 1.5E+07 |
| UGT76G1var28 | L243A_S274G_P382M | 6.1 | 2.4E+06 |
| UGT76G1var29 | F22Y_V279I_N409R | 41.0 | 2.9E+07 |
| UGT76G1var30 | I43V_E224A_S241I | 13.6 | 5.6E+06 |
| UGT76G1var31 | E224D_L243P_V300A | 0.4 | 2.4E+05 |
| UGT76G1var32 | A239V_L243A_S375M | 0.0 | −4.4E+04 |
| UGT76G1var33 | A33G_R334H_Y397Q | 1.0 | 7.5E+06 |
| UGT76G1var34 | I43V_T284I_I295T | 3.4 | 1.5E+06 |
| UGT76G1var35 | T284L_F314S_S357N | 0.5 | 1.8E+05 |
| UGT76G1var36 | F265L_L379A_V396F | 20.0 | 8.8E+06 |
| UGT76G1var37 | E229A_L379G_I407V | 39.1 | 2.8E+07 |
| UGT76G1var38 | F41L_I295M_F377C | 8.2 | 3.7E+06 |
| UGT76G1var39 | F22Y_F41L_V366I | 7.2 | 3.3E+06 |
| UGT76G1var40 | T263S_Q266E_S375R | 47.6 | 3.3E+07 |
| UGT76G1var41 | L246F_A385T_K393G | 0.8 | 1.4E+06 |
| UGT76G1var42 | T263S_Q266S_R334H | 34.6 | 2.2E+07 |
| UGT76G1var43 | S241I_P272A_V279I | 19.9 | 9.4E+06 |
| UGT76G1var44 | I335V_S375R_I407V | 35.3 | 2.3E+07 |
| UGT76G1var45 | V279I_D301N_S389E | 38.6 | 2.3E+07 |
| UGT76G1var46 | F22L_Q266E_I295M | 0.6 | 9.8E+05 |
| UGT76G1var47 | E229A_T284I_S389E | 4.8 | 2.7E+06 |
| UGT76G1var48 | V394I_Y397E_Q432E | 47.6 | 3.8E+07 |
| UGT76G1var49 | F41L_Q266E_T284I_Y397Q | 2.6 | 1.1E+06 |
| UGT76G1var50 | F22Y_V310I_S375M_F377C | 1.9 | 7.9E+05 |
| UGT76G1var51 | K303G_S357C_S389E_V396F | 18.7 | 9.5E+06 |
| UGT76G1var52 | D301N_I373L_F377C_I407V | 12.9 | 4.6E+06 |
| UGT76G1var53 | R334K_A352G_P382M_S389E | 9.3 | 4.1E+06 |
| UGT76G1var54 | E229A_T284L_R334K_Q342E | 0.7 | 4.3E+05 |
| UGT76G1var55 | I295M_Q342E_V366I_N409R | 1.0 | 2.2E+05 |
| UGT76G1var56 | L246F_A352G_S357N_Q432E | 0.4 | 4.1E+04 |
| UGT76G1var57 | S241I_T263S_L379G_A385T | 0.8 | 1.5E+05 |
| UGT76G1var58 | S357C_S375M_N409R_Q425E | 7.5 | 2.2E+06 |
| UGT76G1var59 | I335V_K393G_V394I_Y397Q | 33.0 | 2.7E+07 |
| UGT76G1var60 | E231A_L243A_V279I_S357N | 0.5 | 9.5E+04 |
| UGT76G1var61 | I43V_F265I_Q266S_L379A | 6.4 | 2.0E+06 |
| UGT76G1var62 | L243P_P272A_V394I_V396F | 0.1 | 3.4E+04 |
| UGT76G1var63 | F314S_R334H_Q342K_L379G | 3.4 | 1.2E+06 |
| UGT76G1var64 | F22L_A239I_R334H_I407V | 0.3 | 3.1E+04 |
| UGT76G1var65 | A33G_A239V_P382M_Q425E | 1.2 | 3.3E+05 |
| UGT76G1var66 | F265L_V310I_V366I_A385T | 0.8 | 3.7E+05 |
| UGT76G1var67 | E224D_F314S_S375R_Y397E | −2.1 | −5.6E+05 |
| UGT76G1var68 | Q342K_G348P_I373L_Y397E | −1.4 | −1.1E+05 |
| UGT76G1var69 | S274G_I295T_I335V_L379A | 24.7 | 8.3E+06 |
| UGT76G1var70 | E224A_I295T_V300A_G348P | 24.0 | 8.4E+06 |
| UGT76G1var71 | I295M_V300A_K393G_Q432E | 42.9 | 2.1E+07 |
| UGT76G1var72 | T284L_D301N_K303G_S375R | 19.2 | 9.1E+06 |
| UGT76G1var73 | F22Y_D301N_R334H_Q342E_V396F | 0.8 | 8.7E+05 |
| UGT76G1var74 | I295T_I373L_S375R_Y397Q_Q432E | 0.6 | 9.6E+04 |
| UGT76G1var75 | F41L_A239I_Q266S_S375M_P382M | 0.8 | −1.3E+05 |
| UGT76G1var76 | F22Y_A239I_L246F_I295M_R334K | 2.6 | 7.2E+05 |
| UGT76G1var77 | A239V_F265I_I295T_D301N_K393G | 1.9 | 4.4E+05 |
| UGT76G1var78 | V279I_V300A_V310I_I335V_S357C | 3.2 | 8.2E+05 |
| UGT76G1var79 | E224D_T284I_V366I_I373L_K393G | 8.5 | 3.8E+06 |
| UGT76G1var80 | L243P_L379A_S389E_Q425E_Q432E | 1.0 | 2.1E+05 |
| UGT76G1var81 | A33G_T263S_S274G_V279I_Y397E | 15.0 | 6.5E+06 |
| UGT76G1var82 | E224D_L243A_F265L_R334H_A352G | 1.1 | 2.5E+05 |
| UGT76G1var83 | I43V_Q342E_S357N_S375R_L379G | 0.5 | 4.3E+04 |
| UGT76G1var84 | F22L_Q266S_F314S_A352G_S357C | 1.2 | 2.3E+05 |
| UGT76G1var85 | T284L_G348P_F377C_P382M_N409R | 1.8 | 4.0E+05 |
| UGT76G1var86 | E224A_T284L_V396F_Y397E_I407V | 1.6 | 3.8E+05 |
| UGT76G1var87 | S241I_L243A_V300A_F314S_N409R | 35.7 | 2.1E+07 |
| UGT76G1var88 | A239V_T284I_V310I_Q342K_L379A | 1.6 | 3.8E+05 |
| UGT76G1var89 | F41L_E229A_E231A_F265L_P272A | 1.2 | 2.1E+05 |
| UGT76G1var90 | E231A_S241I_S274G_Y397Q_Q425E | 34.5 | 1.9E+07 |
| UGT76G1var91 | E224A_L246F_T263S_F265I_Q342K | 1.2 | 2.3E+05 |
| UGT76G1var92 | K303G_S357N_V366I_V394I_I407V | 1.6 | 3.6E+05 |
| UGT76G1var93 | I43V_Q266E_S375M_S389E_V394I | 1.8 | 4.5E+05 |
| UGT76G1var94 | Q266E_P272A_R334K_G348P_L379G | 72.0 | 7.9E+07 |
| UGT76G1var95 | A33G_I295M_K303G_I335V_A385T | −1.3 | −1.7E+05 |
| UGT76G1var96 | F22L_E229A_L243P_F377C_A385T | 1.2 | 2.7E+05 |

*Mutations are noted as follows: original amino acid-position-new amino acid: For example the mutation of an alanine at position 33 to a glycine is noted as A33G.

### Example 27

### In-Vivo Production of UGTSL2

UGTSL2 (GI_460410132/XP_004250485.1) amino
acid sequence:
SEQ ID 9
MATNLRVLMFPWLAYGHISPFLNIAKQLADRGFLIYLCSTRINLESIIKK
IPEKYADSIHLIELQLPELPELPPHYHTTNGLPPHLNPTLHKALKMSKPN
FSRILQNLKPDLLIYDVLQPWAEHVANEQNIPAGKLLTSCAAVFSYFFSF
RKNPGVEFPFPAIHLPEVEKVKIREILAKEPEEGGRLDEGNKQMMLMCTS
RTIEAKYIDYCTELCNWKVVPVGPPFQDLITNDADNKELIDWLGTKHENS
TVFVSFGSEYFLSKEDMEEVAFALELSNVNFIWVARFPKGEERNLEDALP
KGFLERIGERGRVLDKFAPQPRILNHPSTGGFISHCGWNSAMESIDFGVP
IIAMPIHNDQPINAKLMVELGVAVEIVRDDDGKIHRGEIAETLKSVVTGE
TGEILRAKVREISKNLKSIRDEEMDAVAEELIQLCRNSNKSK

The pET30A+ vector containing the UGTSL2 gene was introduced in E. coli B121(DE3) by heat shock. The obtained cells were grown in petri-dishes in the presence of Kanamycin and suitable colonies were selected and allowed to grow in liquid LB medium (erlenmeyer flasks). Glycerol was added to the suspension as cryoprotecteur and 400 μL aliquots were stored at −20° C. and at −80° C.

The storage aliquots of E. coli BL21(DE3) containing the pET30A+_UGTSL2 plasmids were thawed and added to 30 mL of LBGKP medium (20 g/L Luria Broth Lennox; 50 mM PIPES buffer pH 7.00; 50 mM Phosphate buffer pH 7.00; 2.5 g/L glucose and 50 mg/L of Kanamycin). This culture was allowed to shake at 135 rpm at 30° C. for 8 h.

The production medium contained 60 g/L of overnight express instant TB medium (Novagen), 10 g/L of glycerol and 50 mg/L of Kanamycin. The preculture was added to 200 mL of this medium and the solution was allowed to stir at 20° C. while taking samples to measure the OD and pH. The culture gave significant growth and a good OD was obtained. After 40 h, the cells were harvested by centrifugation and frozen to obtain 6.22 g of cell wet weight.

Lysis was performed on 1.4 g of cells by addition of Bugbuster Master mix (Novagen) and the lysate was recovered by centrifugation and used fresh.

### Example 28

Determination of Activity for Stevioside to Rebaudioside E Conversion with UGTSL and UGTSL2

### UGTSL was Prepared According to EXAMPLE 22, and UGTSL2 was Prepared According to EXAMPLE 27

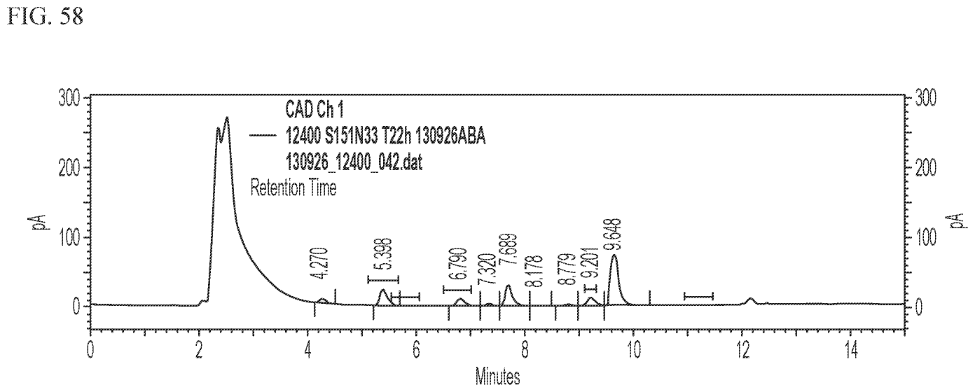

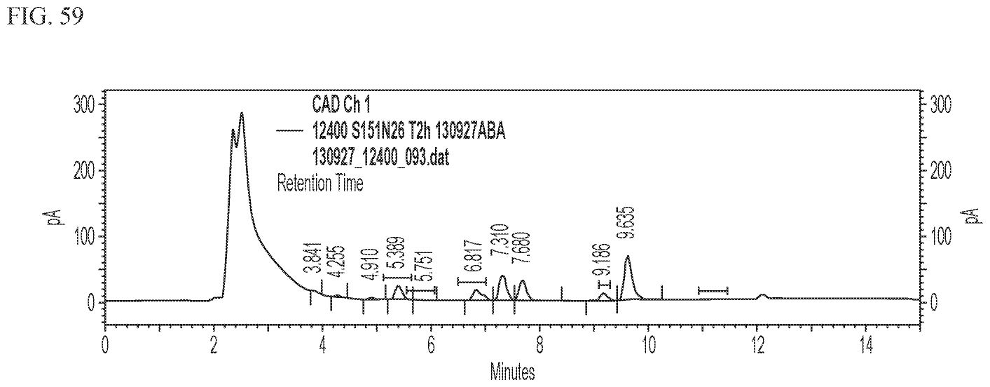

Activity tests were performed at 3 mL scale with 600 μL of lysate for the transformation of Stevioside using 0.5 mM of substrate, 2.5 mM of UDP-Glucose and 3 mM MgCl₂ in 50 mM Sodium Phosphate buffer at pH 7.2. Samples were taken and analyzed by HPLC. HPLC Analysis as shown in FIGS. 58-59. The HPLC assay was performed as described in EXAMPLE 20.

The results for the different enzymes and the corresponding chromatograms are provided below.

| Enzyme internal | | | Stevioside conv.¹ | Rebaudioside E |
|---|---|---|---|---|
| reference | GI Number | Version | (reaction time) | formation¹ |
| UGTSL | 460409128 | XP_004249992.1 | 74% (22 h.) | 46% |
| UGTSL2 | 460410132 | XP_004250485.1 | 77% (2 h.) | 50% |

Note:
¹Based on initial concentration of Stevioside

The below table accompanies FIG. 58.

SAMPLE: 12400 S151N33 T22 h 130926ABA
Gene references: UGTSL (XP_004249992.1)
Filename: 130926_12400_042.dat
CAD Ch 1 Results

| Compound | Retention time | Integration (area) |
|---|---|---|
| Unknown @RT4.27 | 4.270 | 45,634,692 |
| Rebaudioside E | 5.398 | 215,079,800 |
| Unknown @RT6.79 | 6.790 | 11,0326,212 |
| Unknown @RT7.32 | 7.320 | 33,855,010 |
| Unknown @RT7.69 | 7.689 | 271,186,269 |
| Unknown @RT8.18 | 8.178 | 6,003,490 |
| Unknown @RT8.78 | 8.779 | 20,739,231 |
| Stevioside | 9.201 | 114,734,548 |
| Unknown @RT9.65 | 9.648 | 779,225,521 |
| Total | | 1,596,784,773 |

The below table accompanies FIG. 59.

SAMPLE: 12400 S151N26 T2 h 130927ABA
Gene references: UGTSL2 (XP_004250485.1)
Filename: 130927_12400_093.dat
CAD Ch 1 Results

| Compound | Retention time | Integration (area) |
|---|---|---|
| Unknown @RT3.84 | 3.841 | 16,182,482 |
| Unknown @RT4.25 | 4.255 | 20,078,830 |
| Unknown @RT4.91 | 4.910 | 27,630,795 |
| Rebaudioside E | 5.389 | 203,768,956 |
| Unknown @RT5.75 | 5.751 | 8,018,638 |
| Unknown @RT6.82 | 6.817 | 200,959,602 |
| Unknown @RT7.31 | 7.310 | 370188401 |
| Unknown @RT7.68 | 7.680 | 294,963,186 |
| Stevioside | 9.186 | 101,729,292 |
| Unknown @RT9.63 | 9.635 | 727,903,255 |
| Total | | 1,971,423,437 |

### Example 29

Determination of Activity for Rubusoside to Rebaudioside E Conversion with UGTSL and UGTSL2

### UGTSL was Prepared According to EXAMPLE 22, and UGTSL2 was Prepared According to EXAMPLE 27

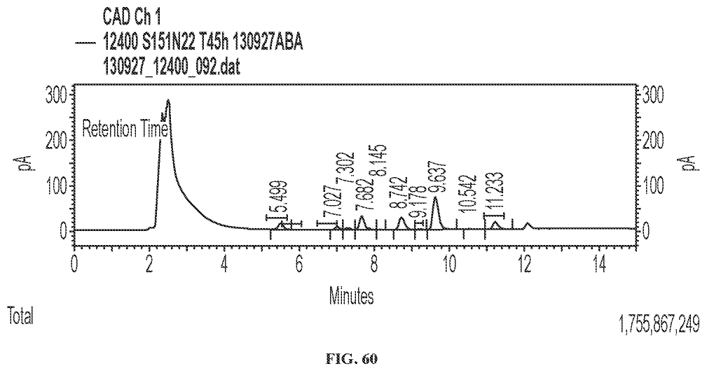

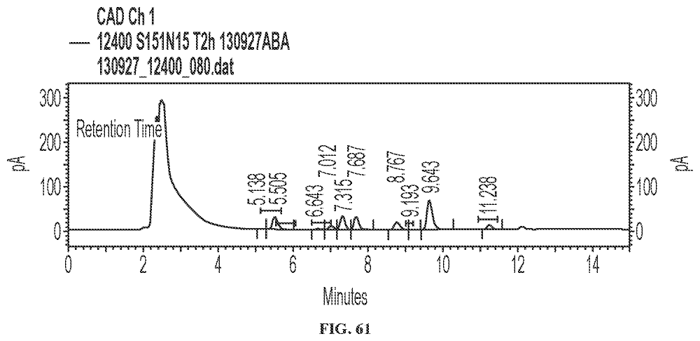

Activity tests were performed at 3 mL scale with 600 μL of lysate for the transformation of Rubusoside using 0.5 mM of substrate, 2.5 mM of UDP-Glucose and 3 mM MgCl₂ in 50 mM Sodium Phosphate buffer at pH 7.2. Samples were taken and analyzed by HPLC as shown in FIGS. 60-61. The HPLC assay was performed as described in EXAMPLE 20.

The results for the different enzymes and the corresponding chromatograms are provided below.

| Enzyme internal | | | Rubusoside conv.¹ | Rebaudioside E |
|---|---|---|---|---|
| reference | GI Number | Version | (reactiontime) | formation |
| UGTSL | 460409128 | XP_004249992.1 | 70% (45 h.) | 27% |
| UGTSL2 | 460410132 | XP_004250485.1 | 80% (2 h.) | 55% |

Note:
¹Based on initial concentration of Rubusoside

SAMPLE: 12400 S151N22 T45 h 130927ABA
Gene references: UGTSL (XP_004249992.1)
Filename: 130927_12400_092.dat
CAD Ch 1 Results

| Compound | Retention time | Integration (area) |
|---|---|---|
| Rebaudioside E | 5.499 | 135,984,743 |
| Unknown @RT7.03 | 7.027 | 54,448,761 |
| Unknown @RT7.30 | 7.302 | 41,308,528 |
| Unknown @RT7.68 | 7.682 | 283,852,603 |
| Unknown @RT8.14 | 8.145 | 5,484,731 |
| Unknown @RT8.74 | 8.742 | 290,946,055 |
| Stevioside | 9.178 | 8,774,098 |
| Unknown @RT9.64 | 9.637 | 761,299,117 |
| Unknown @RT10.54 | 10.542 | 18,276,224 |
| Rubusoside | 11.233 | 155,492,389 |
| Total | | 1,755,867,249 |

SAMPLE: 12400 S151N15 T2 h 130927ABA
Gene references: UGTSL2 (XP_004250485.1)
Filename: 130927_12400_080.dat
CAD Ch 1 Results

| Compound | Retention time | Integration (area) |
|---|---|---|
| Unknown @RT5.14 | 5.138 | 5,555,472 |
| Rebaudioside E | 5.505 | 278,529,547 |
| Unknown @RT6.64 | 6.643 | 23,812,633 |
| Unknown @RT7.01 | 7.012 | 84,543,823 |
| Unknown @RT7.31 | 7.315 | 283,724,517 |
| Unknown @RT7.69 | 7.687 | 264,400,008 |
| Unknown @RT8.78 | 8.767 | 188,634,123 |
| Stevioside | 9.193 | 9,365,107 |
| Unknown @RT9.64 | 9.643 | 700,878,865 |
| Rubusoside | 11.238 | 102,484,386 |
| Totals | | 1,941,928,481 |

### Example 30 Determination of activity for Rebaudioside A to Rebaudioside D conversion with UGTSL2 UGTSL2 was prepared according to EXAMPLE 27

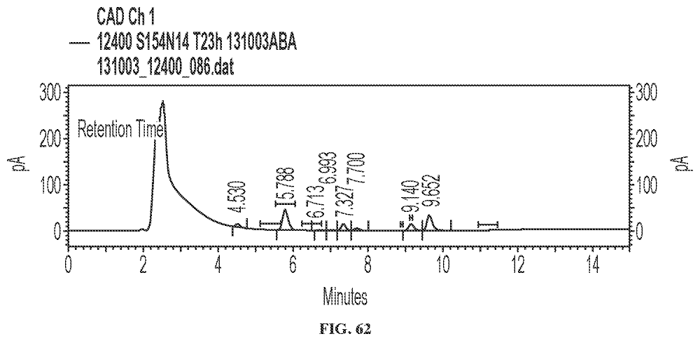

Activity tests were performed at 3 mL scale with 60 μL of lysate for the transformation of Rebaudioside A using 0.5 mM of substrate, 2.5 mM of UDP-Glucose and 3 mM MgCl₂ in 50 mMv Sodium Phosphate buffer at pH 7.2. Samples were taken and analyzed by HPLC as shown in FIG. 62. The HPLC assay was performed as described in EXAMPLE 20.

The result after 23 h. of reaction and the corresponding chromatogram is provided below.

| | | | Rebaudioside | |
|---|---|---|---|---|
| Enzyme | | | A | Rebaudioside |
| internal | GI | | conv.¹ | D |
| reference | Number | Version | (reaction time) | formation¹ |
| UGTSL2 | 460410132 | XP_004250485.1 | 78% (23 h.) | 75% |

Note:
¹Based on initial concentration of Rebaudioside A

The below table accompanies FIG. 62.

SAMPLE: 12400 S154N14 T23 h 131003ABA
Gene references: UGTSL2 (XP_004250485.1)
Filename: 131003_12400_086.dat

| CAD Ch 1 Results | | |
|---|---|---|
| Compound | Retention time | Integration (area) |
| Unknown @RT4.53 | 4.530 | 55,894,278 |
| Rebaudioside D | 5.788 | 461,768,318 |
| Unknown @RT6.71 | 6.713 | 7,942,480 |
| Unknown @RT6.99 | 6.993 | 11,192,896 |
| Unknown @RT7.33 | 7.327 | 120,255,606 |
| Unknown @RT7.70 | 7.700 | 38,994,186 |
| Rebaudioside A | 9.140 | 137,037,966 |
| Unknown @RT9.65 | 9.652 | 314,468,535 |
| Total | | 1,147,554,265 |

### Example 31

### Identification of Glycosides

The reaction mixtures prepared according to EXAMPLE 30 and incubated for 45 hrs was analyzed by LC-MS, along with Stevia rebaudiana Bertoni leaf extract “MLD1” produced by PureCircle Sdn Bhd (Malaysia), to determine the occurrence of formed glycosides in nature.

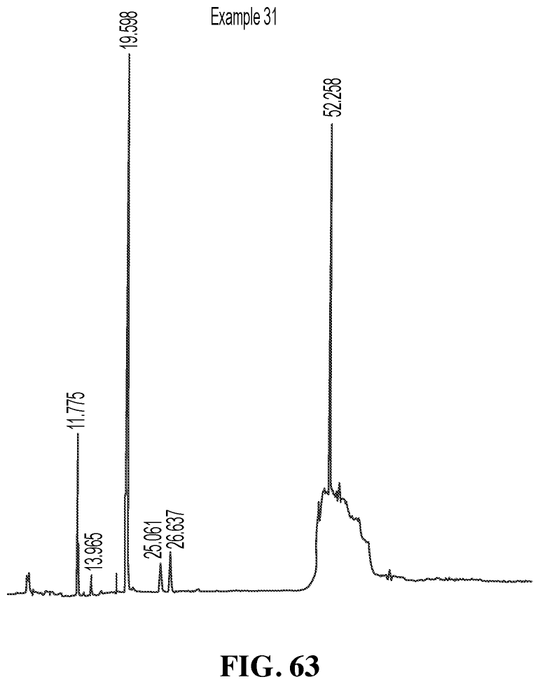

An Agilent 1200 series HPLC system, equipped with binary pump (G1312B), autosampler (G1367D), thermostatted column compartment (G1316B), DAD detector (G1315C), connected with Agilent 6110A MSD, and interfaced with “LC/MSD Chemstation” software, was used, and the chromatogram is shown in FIG. 63.

### Instrument Conditions

| Column | Phenomenex Prodigy 3u C18 100A, | |
|---|---|---|
| | 4.6 mm × 250 mm, 3 μm | |
| Column Temperature | 55° | C. |
| Detection | DAD at 210 nm bw 360 nm | |
| | MSD (Scan and SIM mode) | |
| | Mode: ES-API, Negative Polarity | |
| | Drying gas flow: 13.0 L/min | |
| | Nebulizer pressure: 30 psig | |
| | Drying gas temperature: 270° C. | |
| Analysis duration | 75 | min |
| Injected volume | 10 | μL |
| Flow rate | 0.5 | mL/min |

### Mobile Phase Gradient Program

| Time (min) | A (%): Formic acid 0.1% | B (%): Acetonitrile |
|---|---|---|
| 0 | 75 | 25 |
| 30 | 75 | 25 |
| 33 | 68 | 32 |
| 75 | 68 | 32 |

The assay shows that the compound observed on LC-MS system at 11.77 min is the same as the compound at 3.5 min, in EXAMPLE 23 (C₅₆H₉₀O₃₃; later confirmed as reb M2), and the compound observed at 26.64 min is the same as the compound at 7.6 min, in EXAMPLE 23 (C50H₈₀O₂₈; reb UNK, later confirmed as reb D2). Other isomers of reb M were observed at 13.96 min and also another isomer form of reb D was observed at 25.06 min. All observed compounds occurred in the extract of Stevia rebaudiana Bertoni plant.

The below table accompanies FIG. 63.

SAMPLE: UGTSL2 T45 h
Gene references: UGTSL2 (XP_004250485.1)

| MSD SIM Results | | |
|---|---|---|
| Compound | Retention time | MW |
| Unknown@RT11.77 | 11.775 | 1,291 |
| Unknown@RT13.96 | 13.965 | 1,291 |
| Rebaudioside D | 19.598 | 1,129 |
| Unknown@RT25.06 | 25.061 | 1,129 |
| Unknown@RT26.64 | 26.637 | 1,129 |
| Rebausioside A | 52.258 | 967 |

### Example 32

In vivo preparation and activity determination of UGTLB PGP86

UGTLB (GI_209954733/BAG80557.1) amino
acid sequence
SEQ ID 10
MGTEVTVHKNTLRVLMFPWLAYGHISPFLNVAKKLVDRGFLIYLCSTAIN
LKSTIKKIPEKYSDSIQLIELHLPELPELPPHYHTTNGLPPHLNHTLQKA
LKMSKPNFSKILQNLKPDLVIYDLLQQWAEGVANEQNIPAVKLLTSGAAV
LSYFFNLVKKPGVEFPFPAIYLRKNELEKMSELLAQSAKDKEPDGVDPFA
DGNMQVMLMSTSRIIEAKYIDYFSGLSNWKVVPVGPPVQDPIADDADEME
LIDWLGKKDENSTVFVSFGSEYFLSKEDREEIAFGLELSNVNFIWVARFP
KGEEQNLEDALPKGFLERIGDRGRVLDKFAPQPRILNHPSTGGFISHCGW
NSVMESVDFGVPIIAMPIHLDQPMNARLIVELGVAVEIVRDDYGKIHREE
IAEILKDVIAGKSGENLKAKMRDISKNLKSIRDEEMDTAAEELIQLCKNS
PKLK

The pET30A+ vector containing the UGTLB gene was introduced in E. coli B121(DE3) by heat shock. The obtained cells were grown in petri-dishes in the presence of Kanamycin and suitable colonies were selected and allowed to grow in liquid LB medium (erlenmeyer flasks). Glycerol was added to the suspension as cryoprotecteur and 400 μL aliquots were stored at −20° C. and at −80° C.

The storage aliquots of E. coli BL21(DE3) containing the pET30A+_UGTLB plasmids were thawed and added to 30 mL of LBGKP medium (20 g/L Luria Broth Lennox; 50 mM PIPES buffer pH 7.00; 50 mM Phosphate buffer pH 7.00; 2.5 g/L glucose and 50 mg/L of Kanamycine). This culture was allowed to shake at 135 rpm at 30° C. for 8 h.

The production medium contained 60 g/L of overnight express instant TB medium (Novagen), 10 g/L of glycerol and 50 mg/L of Kanamycine. The preculture was added to 200 mL of this medium and the solution was allowed to stir at 20° C. while taking samples to measure the OD and pH. The culture gave significant growth and a good OD was obtained. After 40 h, the cells were harvested by centrifugation and frozen to obtain 5.7 g of cell wet weight.

Lysis was performed on 1.2 g of cells by addition of 6 mL Bugbuster Master mix (Novagen) and the lysate was recovered by centrifugation and used fresh.

Determination of Activity for Stevioside to Rebaudioside E Conversion with UGTLB

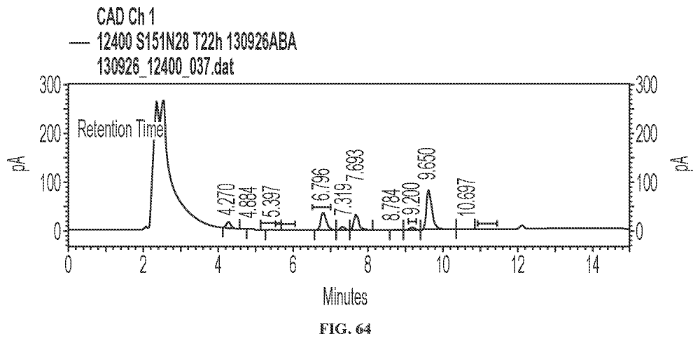

Activity tests were performed at 3 mL scale with 600 μL of lysate for the transformation of Stevioside using 0.5 mM of substrate, 2.5 mM of UDP-Glucose and 3 mM MgCl₂ in 50 mM Sodium Phosphate buffer at pH 7.2. Samples were taken and analyzed by HPLC as shown in FIGS. 64-66. The corresponding chromatograms are depicted in FIGS. 64-66.

| Enzyme | | | Stevioside | Rebaudioside |
|---|---|---|---|---|
| internal | GI | | conv.¹ | E |
| reference | Number | Version | (reaction time) | formation¹ |
| UGTLB | 209954733 | BAG80557.1 | 89% (22 h.) | 3% |

Note:
¹Based on initial concentration of Stevioside

The below table accompanies FIG. 64.

SAMPLE: 12400 S151N28 T22 h 130926ABA
Gene references: UGTLB (BAG80557.1)
Filename: 130926_12400_037.dat

| CAD Ch 1 Results | | |
|---|---|---|
| Compound | Retention time | Integration (area) |
| Unknown@4.27 | 4.270 | 101,580,340 |
| Unknown@4.88 | 4.884 | 2,979,482 |
| Rebaudioside E | 5.397 | 13,747,837 |
| Unknown@6.80 | 6.796 | 378,936,196 |
| Unknown@7.32 | 7.319 | 54,838,779 |
| Unknown@7.69 | 7.693 | 291,189,747 |
| Unknown@8.78 | 8.784 | 21,079,018 |
| Stevioside | 9.200 | 50,143,248 |
| Unknown@9.65 | 9.650 | 888,211,556 |
| Unknown@10.70 | 10.697 | 5,878,160 |
| Totals | | 1,808,584,363 |

Determination of Activity for Rubusoside to Rebaudioside E Conversion with UGTLB

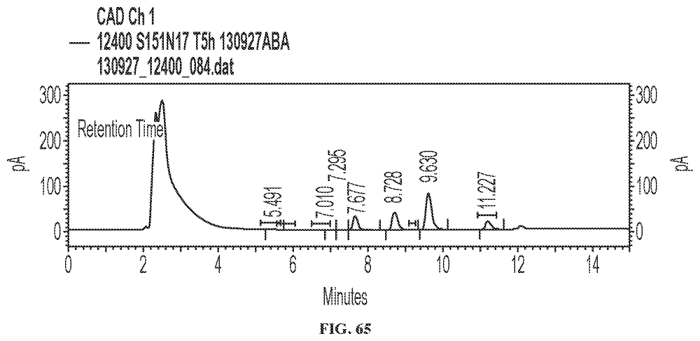

Activity tests were performed at 3 mL scale with 600 μL of lysate for the transformation of Rubusoside using 0.5 mM of substrate, 2.5 mM of UDP-Glucose and 3 mM MgCl₂ in 50 mM Sodium Phosphate buffer at pH 7.2. Samples were taken and analyzed by HPLC. The corresponding chromatogram is depicted in FIG. 65.

| Enzyme | | | Rubusoside | Rebaudioside |
|---|---|---|---|---|
| internal | GI | | conv.¹ | E |
| reference | Number | Version | (reaction time) | formation¹ |
| UGTLB | 209954733 | BAG80557.1 | 65% (5 h.) | 4% |

Note:
¹Based on initial concentration of Rubusoside

The below table accompanies FIG. 65.

SAMPLE: 12400 S151N17 T5 h 130927ABA
Gene references: UGTLB (BAG80557.1)
Filename: 130927_12400_084.dat

| CAD Ch 1 Results | | |
|---|---|---|
| Compound | Retention time | Integration (area) |
| Rebaudioside E | 5.491 | 21921232 |
| Unknown@7.01 | 7.010 | 9764063 |
| Unknown@7.29 | 7.295 | 12510947 |
| Unknown@7.68 | 7.677 | 283386906 |
| Unknown@8.73 | 8.728 | 402240506 |
| Unknown@9.63 | 9.630 | 878990745 |
| Rubusoside | 11.227 | 176000085 |
| Totals | | 1784814484 |

Determination of Activity for Rebaudioside a to Rebaudioside D Conversion with UGTLB

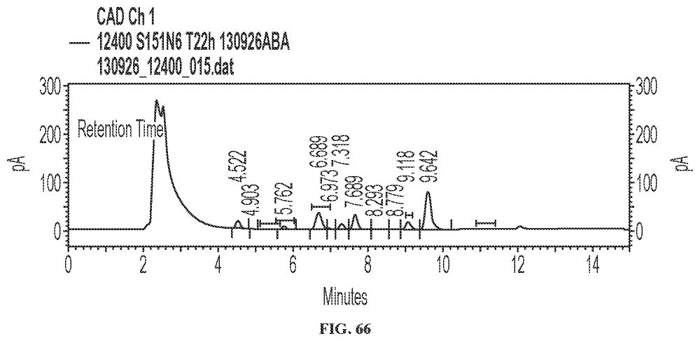

Activity tests were performed at 3 mL scale with 600 μL of lysate for the transformation of Rebaudioside A using 0.5 mM of substrate, 2.5 mM of UDP-Glucose and 3 mM MgCl₂ in 50 mM Sodium Phosphate buffer at pH 7.2. Samples were taken and analyzed by HPLC. The corresponding chromatogram after 23 h. of reaction is depicted in FIG. 66.

| | | | Rebaudioside | |
|---|---|---|---|---|
| Enzyme | | | A | Rebaudioside |
| internal | GI | | conv.¹ | D |
| reference | Number | Version | (reaction time) | formation¹ |
| UGTLB | 209954733 | BAG80557.1 | 72% (22 h.) | 10% |

Note:
¹Based on initial concentration of Rebaudioside A

The below table accompanies FIG. 66.

SAMPLE: 12400 S154N6 T22 h 130926ABA
Gene references: UGTLB (BAG80557.1)
Filename: 130926_12400_015.dat

| CAD Ch 1 Results | | |
|---|---|---|
| Compound | Retention time | Integration (area) |
| Unknown@4.42 | 4.522 | 137,916,950 |
| Unknown@4.90 | 4.903 | 2,015,271 |
| Rebaudioside D | 5.762 | 59,876,764 |
| Unknown@6.69 | 6.689 | 364,185,331 |
| Unknown@6.97 | 6.973 | 26,368,965 |
| Unknown@7.32 | 7.318 | 110,284,197 |
| Unknown@7.69 | 7.689 | 294,579,799 |
| Unknown@8.29 | 8.293 | 7,867,452 |
| Unknown@8.78 | 8.779 | 15,928,550 |
| Rebausioside A | 9.118 | 165,602,247 |
| Unknown@9.64 | 9.642 | 868,327,712 |
| Totals | | 2,052,953,238 |

### Example 33

Determination of reaction products for Rubusoside and Stevioside conversion with UGTSL, UGTSL2, and UGTLB

Conversion of stevioside with UGTSL and UGTSL2 was conducted in similar manner to Example 28, and the conversion of rubusoside with UGTSL and UGTSL2 was conducted similarly to Example 29. Conversions of rubusoside and stevioside with UGTLB was conducted similarly to Example 32.

The reaction mixtures were analyzed by LCMS to determine all reaction products.

Rubusoside conversion products

| | | LC-MS, peak area ratio (%) | | | | | | |
|---|---|---|---|---|---|---|---|---|
| | | | | | | Unknown peak | Unknown peak | Unknown peak |
| | UGT (reaction | | | | | #1 (MW804) | #2 (MW804) | #3 (MW804) |
| Sample ID | time) | Rub | Stev | Reb E | Reb D | RT 30.70 min | RT 49.50 min | RT 50.40 min |
| S151N15 | UGTSL2 (2 hrs) | 3.54 | 2.12 | 52.88 | 6.73 | 12.02 | 9.94 | 12.77 |
| S151N17 | UGTLB (5 hrs) | 13.49 | ND | 9.21 | 1.29 | 4.07 | 66.67 | 5.27 |
| S151N22 | UGTSL (45 hrs) | 7.82 | 2.37 | 35.88 | 3.45 | 20.38 | 27.75 | 2.35 |

Stevioside conversion products

| | | LC-MS, peak area ratio (%) | | | | | |
|---|---|---|---|---|---|---|---|
| | | | | | Unknown peak | Unknown peak | Unknown peak |
| | UGT (reaction | | | | #1 (MW966) | #2 (MW966) | #3 (MW966) |
| Sample ID | time) | Stev | Reb E | Reb D | RT = 22.60 min | RT = 26.50 min | RT = 29.50 min |
| S151N26 | UGTSL2 (2 hrs) | 20.01 | 42.56 | 1.70 | 4.48 | 5.56 | 25.70 |
| S151N28 | UGTLB (2 hrs) | 43.11 | 3.12 | ND | ND | 53.78 | ND |
| S151N33 | UGTSL (22 hrs) | 25.24 | 49.68 | 0.54 | 3.97 | 20.56 | ND |

It can be seen that amongst Rubusoside conversion products, besides Stevioside, Reb E and Reb D, there are at least 3 additional compounds with Molecular Weight of 804. The retention time of these compounds do not match with Reb B which is known to have same Molecular Weight as Stevioside. Since these compounds have same molecular weight with Stevioside it can be assumed that these novel steviol glycosides are isomers of Stevioside.

On the other hand amongst Stevioside conversion products, besides Reb E and Reb D, there are at least 3 additional compounds with Molecular Weight of 966. The retention time of these compounds do not match with Reb A which is known to have same Molecular Weight as Reb E. Since these compounds have same molecular weight with Reb A and Reb E it can be assumed that these novel steviol glycosides are isomers of Reb A (Reb E).

### Example 34

In vivo production of UGT76G1 in S. cerevisiae

UGT76G1 [Stevia rebaudiana] (gi_37993653/
gb_AAR06912.1)
SEQ ID 11
MENKTETTVRRRRRIILFPVPFQGHINPILQLANVLYSKGFSITIFHTNF
NKPKTSNYPHFTFRFILDNDPQDERISNLPTHGPLAGMRIPIINEHGADE
LRRELELLMLASEEDEEVSCLITDALWYFAQSVADSLNLRRLVLMTSSLF
NFHAHVSLPQFDELGYLDPDDKTRLEEQASGFPMLKVKDIKSAYSNWQIL
KEILGKMIKQTKASSGVIWNSFKELEESELETVIREIPAPSFLIPLPKHL
TASSSSLLDHDRTVFQWLDQQPPSSVLYVSFGSTSEVDEKDFLEIARGLV
DSKQSFLWVVRPGFVKGSTWVEPLPDGFLGERGRIVKWVPQQEVLAHGAI
GAFWTHSGWNSTLESVCEGVPMIFSDFGLDQPLNARYMSDVLKVGVYLEN
GWERGEIANAIRRVMVDEEGEYIRQNARVLKQKADVSLMKGGSSYESLES
LVSYISSL

The above mentioned amino acid sequence was codon optimized for expression in S. cerevisiae. Furthermore the yeast consensus sequence AACACA was added before the ATG start codon. The synthetic gene was subcloned in the pYES2 vector using Hind III and Xba I restriction sites. The pYES2_UGT76G1_Sc vector was used to transform chemically competent S. cerevisiae INVScl cells (Invitrogen).

The cells were grown on a solid synthetic minimal medium containing 2% glucose lacking Uracil and a single colony was picked and allowed to grow in liquid synthetic minimal medium lacking Uracil (SC-U containing 2% glucose). After centrifugation, the cells were suspended with SC-U (containing 2% glucose) and 60% glycerol/water. Aliquots were stored at −80° C. and one aliquot was used to start a culture in SC-U (containing 2% glucose) for 43 h at 30° C. Part of this culture was centrifuged and suspended in induction medium (SC-U containing 2% galactose) for 19h30 at 30° C.

Cells were obtained by centrifugation and lysis with five volumes of CelLytic™ Y Cell Lysis Reagent (Sigma). The lysates were used directly for activity testing (UGT76G1_Sc).

### Example 35

### Determination of Activity of UGT76G1_Sc for the Conversion of Rebaudioside D to Rebaudioside M

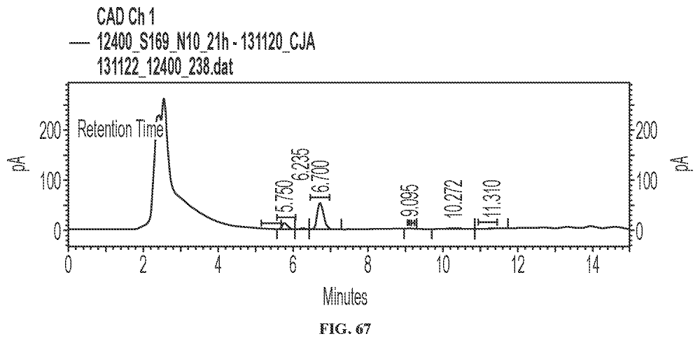

UGT76G1_Sc was prepared according to EXAMPLE 34. Activity tests were performed at 2 mL scale with 200 μL of lysate for the transformation of Rebaudioside D using 0.5 mM of substrate, 2.5 mM of UDP-Glucose and 3 mM MgCl₂ in 50 mM Sodium Phosphate buffer at pH 7.2. Samples were taken and analyzed by HPLC as shown in FIG. 67. The corresponding chromatogram is depicted in FIG. 67.

| Enzyme internal | Rebaudioside D | Rebaudioside M |
|---|---|---|
| reference | conv.¹ (reaction time) | selectivity¹ |
| UGT76G1_Sc | 85% (21 h.) | 100% |

Note:
¹Based on initial concentration of Rebaudioside D

The below table accompanies FIG. 67.

SAMPLE: 12400 S169N10 T21h 131119CJA
Gene references: UGT76G1_Sc
Filename: 131122_12400_238.dat

| CAD Ch 1 Results | | |
|---|---|---|
| Compound | Retention time | Integration (area) |
| Rebaudioside D | 5.750 | 112,094,430 |
| Unknown@6.23 | 6.235 | 17,886,043 |
| Rebaudioside M | 6.700 | 616,583,935 |
| Rebaudioside A | 9.095 | 11,183,884 |
| Unknown@10.27 | 10.272 | 62,863,156 |
| Unknown@11.31 | 11.310 | 35,839,478 |
| Total | | 856,450,926 |

### Example 36

In vivo production of UGTSL in S. cerevisiae

UGTSL [Solanum lycopersicum] (gi_460409128/
XP_004249992.1
SEQ ID 12
MSPKLHKELFFHSLYKKTRSNHTMATLKVLMFPFLAYGHISPYLNVAKKL
ADRGFLIYFCSTPINLKSTIEKIPEKYADSIHLIELHLPELPQLPPHYHT
TNGLPPNLNQVLQKALKMSKPNFSKILQNLKPDLVIYDILQRWAKHVANE
QNIPAVKLLTSGAAVFSYFFNVLKKPGVEFPFPGIYLRKIEQVRLSEMMS
KSDKEKELEDDDDDDDLLVDGNMQIMLMSTSRTIEAKYIDFCTALTNWKV
VPVGPPVQDLITNDVDDMELIDWLGTKDENSTVFVSFGSEYFLSKEDMEE
VAFALELSNVNFIWVARFPKGEERNLEDALPKGFLERIGERGRVLDKFAP
QPRILNHPSTGGFISHCGWNSAMESIDFGVPIIAMPMHLDQPMNARLIVE
LGVAVEIVRDDDGKIHRGEIAETLKGVITGKTGEKLRAKVRDISKNLKTI
RDEEMDAAAEELIQLCRNGN

The above mentioned amino acid sequence was codon optimized for expression in S. cerevisiae. Furthermore the yeast consensus sequence AACACA was added before the ATG start codon. The synthetic gene was subcloned in the pYES2 vector using Hind III and Xba I restriction sites. The pYES2_UGTSL_Sc vector was used to transform chemically competent S. cerevisiae INVScl cells (Invitrogen).

The cells were grown on a solid synthetic minimal medium containing 2% glucose, lacking Uracil and a single colony was picked and allowed to grow in liquid synthetic minimal medium lacking Uracil (SC-U containing 2% glucose). After centrifugation, the cells were suspended with SC-U (containing 2% glucose) and 60% glycerol/water. Aliquots were stored at −80° C. and one aliquot was used to start a culture in SC-U (containing 2% glucose) for 43 h at 30° C. Part of this culture was centrifuged and suspended in induction medium (SC-U containing 2% galactose) for 19h30 at 30° C. Cells were obtained by centrifugation and lysis with five volumes of CelLytic™ Y Cell Lysis Reagent (Sigma). The lysates were used directly for activity testing (UGTSL_Sc).

### Example 37

### Determination of Activity of UGTSL_Sc for the Conversion of Rebaudioside a to Rebaudioside D

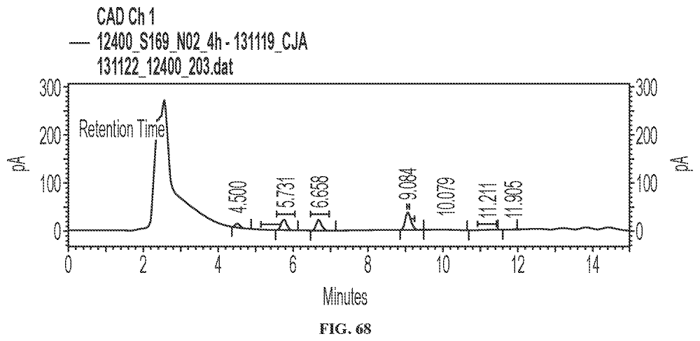

UGTSL_Sc was prepared according to EXAMPLE 36. Activity tests were performed at 2 mL scale with 200 μL of lysate for the transformation of Rebaudioside A using 0.5 mM of substrate, 2.5 mM of UDP-Glucose and 3 mM MgCl₂ in 50 mM Sodium Phosphate buffer at pH 7.2. Samples were taken and analyzed by HPLC as shown in FIG. 68. The corresponding chromatogram is depicted in FIG. 68.

| Enzyme internal | Rebaudioside A | Rebaudioside D |
|---|---|---|
| reference | conv.¹ (reaction time) | selectivity¹ |
| UGTSL_Sc | 46% (4 h) | 42% |

Note:
¹Based on initial concentration of Rebaudioside A

The below table accompanies FIG. 68.

SAMPLE: 12400 S169N02 T4 h 131119CJA
Gene references: UGTSL_Sc
Filename: 131122_12400_203.dat

| CAD Ch 1 Results | | |
|---|---|---|
| Compound | Retention time | Integration (area) |
| Unknown@4.50 | 4.500 | 75,046,986 |
| Rebaudioside D | 5.731 | 223,409,643 |
| Unknown@6.66 | 6.658 | 228,651,278 |
| Rebaudioside A | 9.084 | 404,642,305 |
| Unknown@10.08 | 10.079 | 43,992,253 |
| Unknown@11.21 | 11.211 | 29,776,761 |
| Unknown@11.90 | 11.905 | 2,185,316 |
| Total | | 1,007,704,542 |

### Example 38

### Isolation of Rebaudioside M

The amount of the product mixture of Example 14 was not large enough to separate via preparative HPLC methods. Accordingly, analytical HPLC with a series of injections was used to separate the components of the mixture. Separation was conducted according to the method described above in Example 14 to provide two fractions corresponding to the two main peaks in the HPLC trace of FIG. 5:Fraction A (retention time 24.165 minutes) and Fraction B (retention time 31.325 minutes).

The retention time of Fraction A was consistent with reb D, indicating unreacted starting material from the biotransformation reaction.

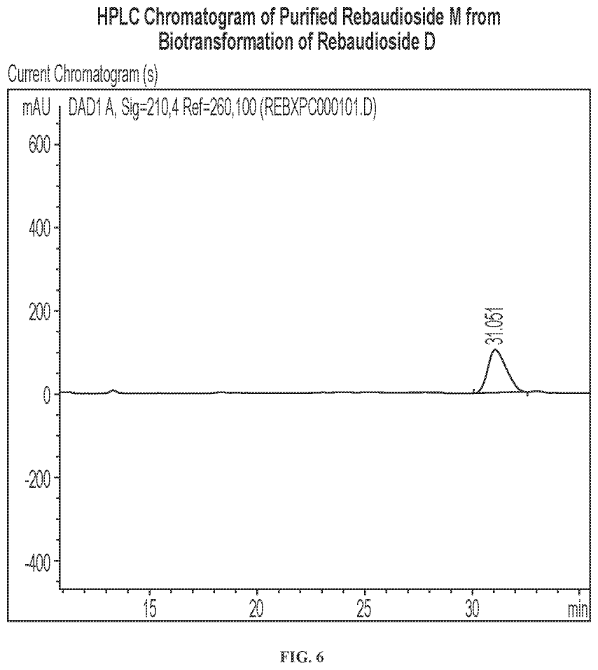

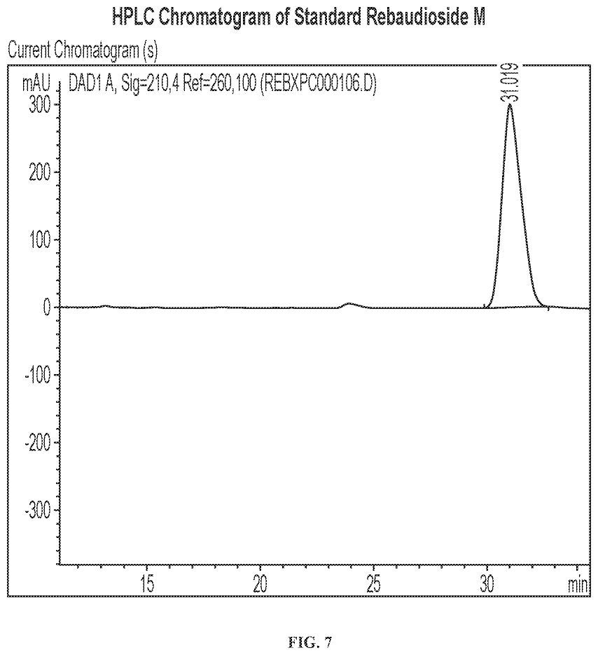

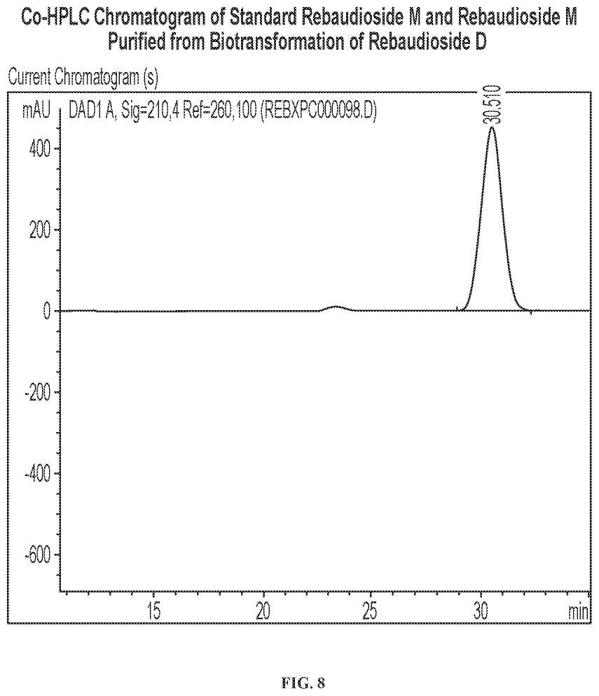

The retention time of purified Fraction B (FIG. 6) was consistent with reb M, indicating successful biotransformation from reb D. The identity of the material collected in Fraction B as reb M was confirmed by co-injection of purified Fraction B with a reb M standard (available from PureCircle, HPLC trace of reb M standard shown in FIG. 7). Both Fraction B and the reb M standard were found to elute at the same retention time (FIG. 8), indicating Fraction B was reb M.

The identity of Fraction B as reb M was also separately confirmed by NMR and HRMS. For sampling, Fraction B was concentrated under rotary evaporator, freeze dried and dried for 40 h at 40° C.

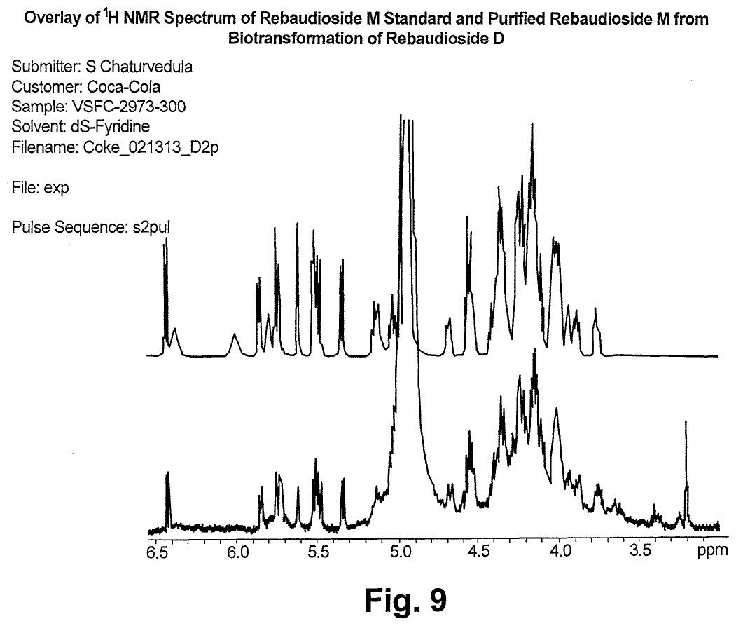

The NMR sample was dissolved in deuterated pyridine (C5D5N) and spectra were acquired on a Varian Unity Plus 600 MHz instrument using standard pulse sequences. The NMR spectra of Fraction B was compared to the NMR spectra of reb M. An overlay of the two spectra (FIG. 9) showed consistency of peaks of Fraction B with reb M. A table of the NMR assignments for reb M is shown below:

¹H and ¹³C NMR spectral data for Rebaudioside M in C₅D₅N ^{a-c}.

| Position | ¹³C NMR | ¹H NMR |
|---|---|---|
| 1 | 40.3 | 0.75 t (13.2) |
| | | 1.76 m |
| 2 | 19.6 | 1.35 m |
| | | 2.24 m |
| 3 | 38.4 | 1.01 m |
| | | 2.30 d (13.3) |
| 4 | 44.3 | — |
| 5 | 57.4 | 1.06 d (12.8) |
| 6 | 23.5 | 2.23 m |
| | | 2.41 q (13.2) |
| 7 | 42.6 | 1.41 m |
| | | 1.80 m |
| 8 | 41.2 | — |
| 9 | 54.3 | 0.91 d (7.7) |
| 10 | 39.7 | — |
| 11 | 20.2 | 1.65 m |
| | | 1.75 m |
| 12 | 38.5 | 1.86 m |
| | | 2.73 m |
| 13 | 87.6 | — |
| 14 | 43.3 | 2.02 m |
| | | 2.74 m |
| 15 | 46.5 | 1.88 d (16.4) |
| | | 2.03 m |
| 16 | 153.3 | — |
| 17 | 104.9 | 4.90 s |
| | | 5.69 s |
| 18 | 28.2 | 1.32 s |
| 19 | 176.9 | — |
| 20 | 16.8 | 1.38 s |
| 1′ | 94.9 | 6.39 d (8.2) |
| 2′ | 76.9 | 4.51 t (8.5) |
| 3′ | 88.6 | 5.09 t (8.5) |
| 4′ | 70.1 | 4.18 m |
| 5′ | 78.4 | 4.13 m |
| 6′ | 61.8 | 4.20 m |
| | | 4.31 m |
| 1″ | 96.2 | 5.46 d (7.1) |
| 2″ | 81.4 | 4.13 m |
| 3″ | 87.9 | 4.98 t (8.5) |
| 4″ | 70.4 | 4.07 t (9.6) |
| 5″ | 77.7 | 3.94 m |
| 6″ | 62.6 | 4.19 m |
| | | 4.32 m |
| 1′′′ | 104.8 | 5.48 d (7.7) |
| 2′′′ | 75.8 | 4.15 m |
| 3′′′ | 78.6 | 4.13 m |
| 4′′′ | 73.2 | 3.98 m |
| 5′′′ | 77.6 | 3.74 ddd (2.8, 6.4, 9.9) |
| 6′′′ | 64.0 | 4.27 m |
| | | 4.51 m |
| 1′′′′ | 103.9 | 5.45 d (7.5) |
| 2′′′′ | 75.6 | 3.98 m |
| 3′′′′ | 77.8 | 4.50 t (7.8) |
| 4′′′′ | 71.3 | 4.14 m |
| 5′′′′ | 78.0 | 3.99 m |
| 6′′′′ | 62.1 | 4.20 m |
| | | 4.32 m |
| 1′′′′′ | 104.2 | 5.81 d (7.2) |
| 2′′′′′ | 75.5 | 4.20 m |
| 3′′′′′ | 78.4 | 4.20 m |
| 4′′′′′ | 73.6 | 4.10 m |
| 5′′′′′ | 77.8 | 3.90 ddd (2.8, 6.4, 9.9) |
| 6′′′′′ | 64.0 | 4.32 m |
| | | 4.64 d (10.3) |
| 1′′′′′′ | 104.1 | 5.31 d (8.0) |
| 2′′′′′′ | 75.5 | 3.95 m |
| 3′′′′′′ | 78.0 | 4.37 t (9.1) |
| 4′′′′′′ | 71.1 | 4.10 m |
| 5′′′′′′ | 78.1 | 3.85 ddd (1.7, 6.1, 9.9) |
| 6′′′′′′ | 62.1 | 4.10 m |
| | | 4.32 m |

^{a} assignments made on the basis of COSY, HMQC and HMBC correlations;
^{b} Chemical shift values are in δ (ppm);
^{c} Coupling constants are in Hz.

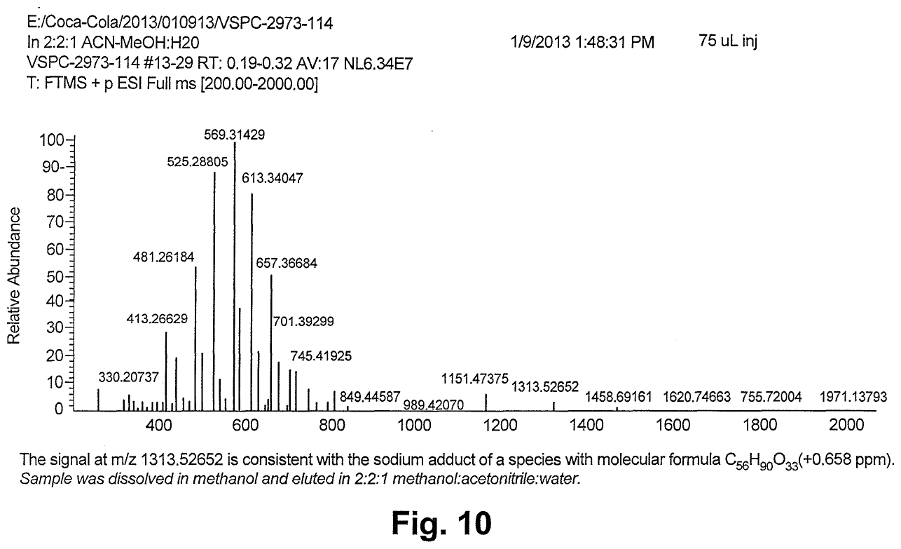

HRMS (FIG. 10) was generated with a Waters Premier Quadropole Time-of-Flight (Q-TOF) mass spectrometer equipped with an electrospray ionization source operated in the positive-ion mode. The sample was dissolved in methanol and eluted in 2:2:1 methanol:acetonitrile:water and introduced via infusion using the onboard syringe pump. The presence of reb M was confirmed by a [M+Na]⁺ adduct at m/z 1313.5265, which corresponds to a molecular formula of C₅₆H₉₀O₃₃

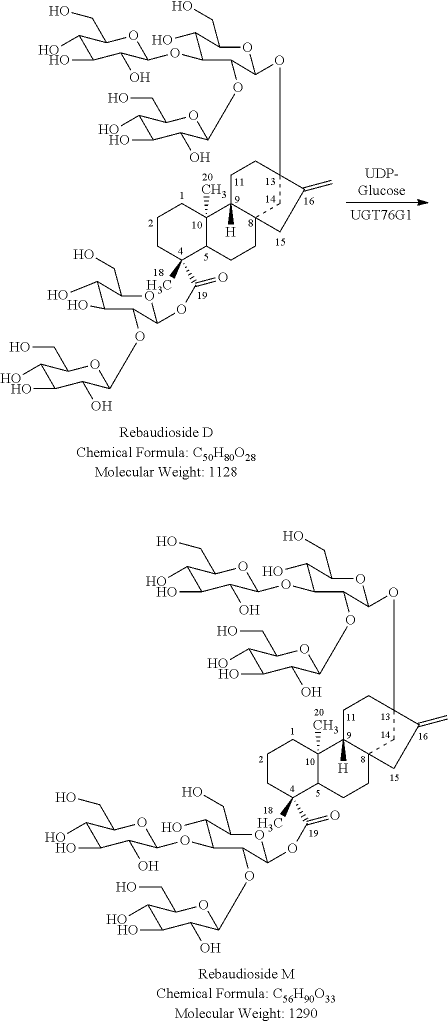

### Example 39

### Isolation and Characterization of Reb D2

Crude Reaction Sample. The sample, Lot CB-2977-106, used for isolation, was prepared according to Example 22 with UGTSL (GI #460409128).

HPLC Analysis. Preliminary HPLC analyses of samples were performed using a Waters 2695 Alliance System with the following method: Phenomenex Synergi Hydro-RP, 4.6×250 mm, 4 μm (p/n 00G-4375-E0); Column Temp: 55° C.; Mobile Phase A: 0.0284% ammonium acetate (NH₄OAc) and 0.0116% acetic acid (HOAc) in water; Mobile Phase B: Acetonitrile (MeCN); Flow Rate: 1.0 mL/min; Injection volume: 10 μL. Detection was by UV (210 nm) and CAD.

Gradient:

| Time (min) | % A | % B |
|---|---|---|
| 0.0-8.5 | 75 | 25 |
| 10.0 | 71 | 29 |
| 16.5 | 70 | 30 |
| 18.5-24.5 | 66 | 34 |
| 26.5-29.0 | 48 | 52 |
| 31-37 | 30 | 70 |
| 38 | 75 | 25 |

Analyses of semi-preparative purification fractions were performed with the following method: Waters Atlantis dC18, 4.6×100 mm, 5 μm (p/n 186001340); Mobile Phase A: 25% MeCN in water; Mobile Phase B: 30% MeCN in water; Flow Rate: 1.0 mL/min; Injection volume: 10 μL. Detection was by CAD.

Gradient:

| Time (min) | % A | % B |
|---|---|---|
| 0.0-5.0 | 100 | 0 |
| 20 | 20 | 80 |
| 25 | 20 | 80 |
| 30 | 100 | 0 |

LC-MS. Preliminary analysis of the semi-synthetic steviol glycoside mixture was carried out on a Waters AutoPurification HPLC/MS System with a Waters 3100 Mass Detector operating in negative ion mode. Analysis of the sample was performed using the following method: Phenomenex Synergi Hydro-RP, 4.6×250 mm, 4 μm (p/n 00G-4375-E0); Column Temp: 55° C.; Mobile Phase A: 0.0284% NH₄OAc and 0.0116% HOAc in water; Mobile Phase B: Acetonitrile; Flow Rate: 1.0 mL/min; Injection volume: 10 μL. Detection was by UV (210 nm), and MSD (−ESI m/z 500-2000). Gradient conditions were as listed above.

Isolation by HPLC. The purification was performed in two steps. The first method used for the semi-preparative purification is summarized below. Column: Waters Atlantis dC18, 30×100 mm, 5 μm (p/n 186001375); Mobile Phase A: 25% MeCN in water; Mobile Phase B: 30% MeCN in water; Flow Rate: 45 mL/min; Injection load: 160 mg dissolved in 20 mL of water. Detection was by UV (205 nm).

Gradient:

| Time (min) | % A | % B |
|---|---|---|
| 0.0-5.0 | 100 | 0 |
| 20 | 20 | 80 |
| 25 | 20 | 80 |
| 30 | 100 | 0 |

The secondary purification used the same column and conditions, but isocratic mobile phase: 20% MeCN in water.

Purification from Natural Extracts. The purification was performed in three steps. The first method used for the preparative purification is summarized below. Primary Process: Waters Symmetry C18, 50×250 mm, 7 μm (p/n WAT248000); Isocratic mobile phase: 50% methanol (MeOH) in water with 0.05% HOAc; Flow Rate: 85 mL/min; Injection load: 6 g crude extract dissolved in 50 mL of mobile phase. Detection was by UV (210 nm). Following the elution of target analytes, the column was flushed with 85% MeOH in water.

Secondary Process: Waters Symmetry Shield RP18, 50×250 mm, 7 μm (p/n WAT248000); Isocratic mobile phase: 20% MeCN in water; Flow Rate: 100 mL/min; Injection load: 0.5 g primary fraction dissolved in 30 mL of water. Detection was by UV (210 nm).

Tertiary Process: Waters Symmetry Shield RP18, 50×250 mm, 7 μm (p/n WAT248000); Isocratic mobile phase: 20% MeCN in water; Flow Rate: 100 mL/min; Injection load: 0.5 g secondary fraction dissolved in 30 mL of water. Detection was by UV (210 nm).

MS and MS/MS. MS and MS/MS data were generated with a Waters QT of Premier mass spectrometer equipped with an electrospray ionization source. Samples were analyzed by negative ESI. Samples were diluted with H₂O:acetonitrile (1:1) by 50 fold and introduced via infusion using the onboard syringe pump. The samples were diluted to yield good s/n which occurred at an approximate concentration of 0.01 mg/mL.

NMR. The sample was prepared by dissolving 1-2 mg in 150 μL of pyridine-d₅ and NMR data were acquired on a Bruker Avance 500 MHz instrument with a 2.5 mm inverse detection probe. The ¹H NMR spectrum was referenced to the residual solvent signal (SH 8.74 and δ_{C} 150.35 for pyridine-d₅).

Results and Discussion

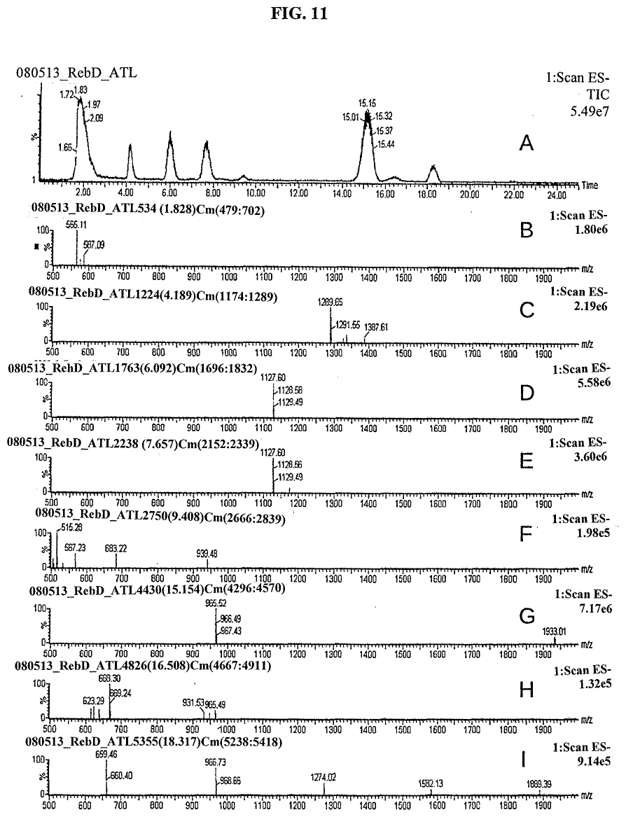

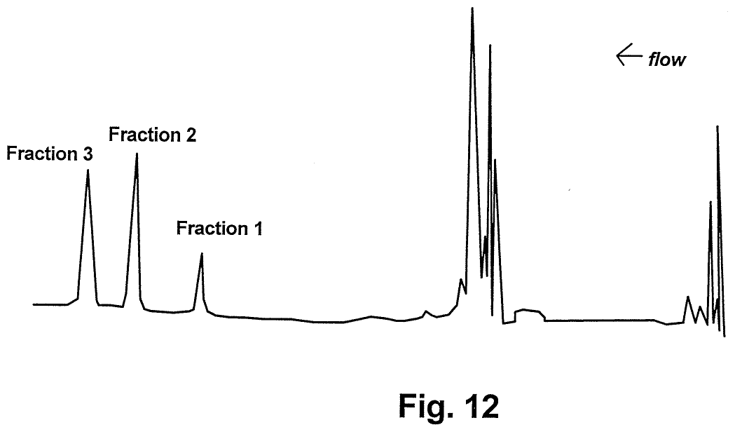

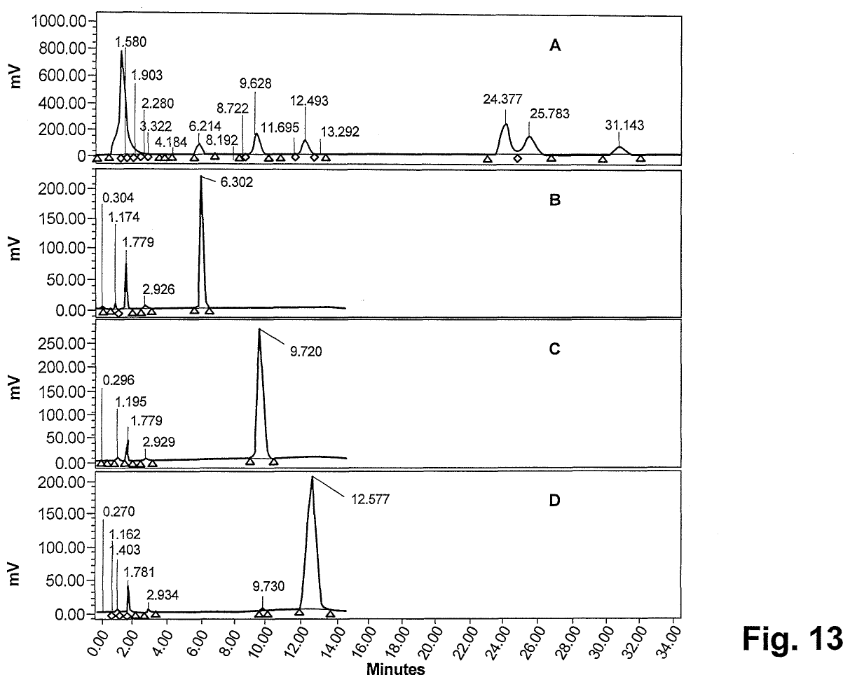

Isolation and Purification. Isolation was performed on steviol glycoside mixture, Lot number CB-2977-106, prepared according to Example 22 with UGTSL (GI #460409128) The material was analyzed by LC-MS using the method described above and results are provided in FIG. 11. The targeted peak of interest was that at 7.7 min in the TIC chromatogram. The mass spectrum of this peak provided a [M-H]⁻ ion at m z 1127.6. The provided sample was preliminarily processed in a single injection (160 mg) using the first method condition provided above. This method fractionated the material into ‘polar’ and ‘non-polar’ mixtures of glycosides. The ‘polar’ mixture was then reprocessed using the second-step conditions above. The semi-preparative HPLC trace is provided in FIG. 12. From this semi-preparative collection, the compound was isolated with a purity>99% (CAD, AUC). The fraction analysis is provided in FIG. 13. Following the purification, the combined fractions were concentrated by rotary evaporation at 35° C. and lyophilized. Approximately 1-2 mg was obtained for characterization.

Mass Spectrometry. The ESI- TOF mass spectrum acquired by infusing a sample showed a [M-H]-ion at m z 1127.4709. The mass of the [M-H]⁻ ion was in good agreement with the molecular formula C₅₀H₈₀O₂₈ (calcd for C50H₇₉O₂₈: 1127.4758, error: −4.3 ppm). The MS data confirmed a nominal mass of 1128 Daltons with the molecular formula, C₅₀H₈₀O₂₈.

The MS/MS spectrum (selecting the [M-H]⁻ ion at m z 1127.5 for fragmentation) indicated the loss of two glucose units and sequential loss of three glucose moieties at m z 641.3187, 479.2655 and 317.2065.

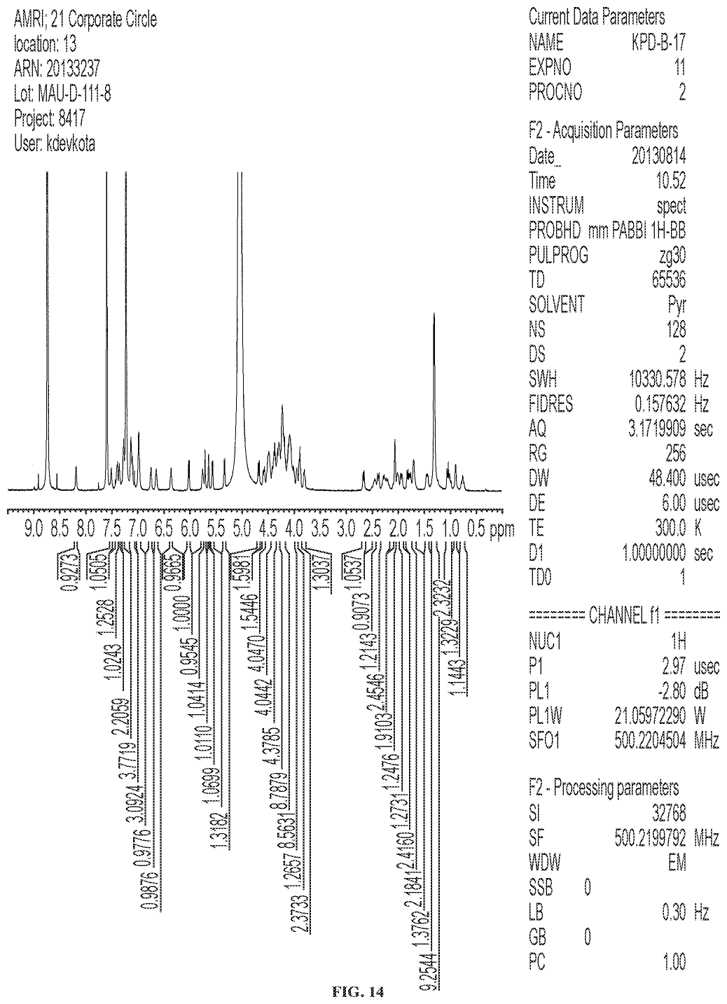

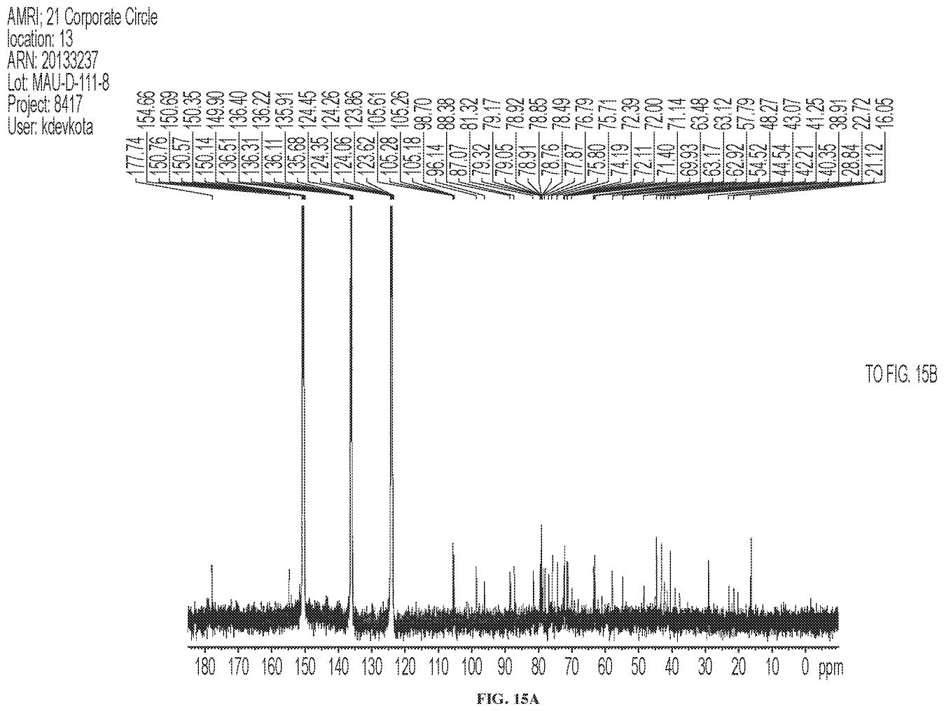

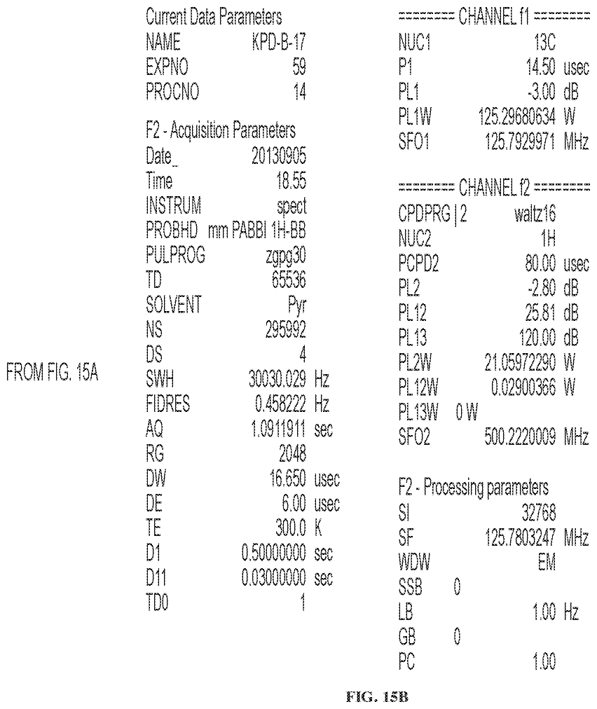

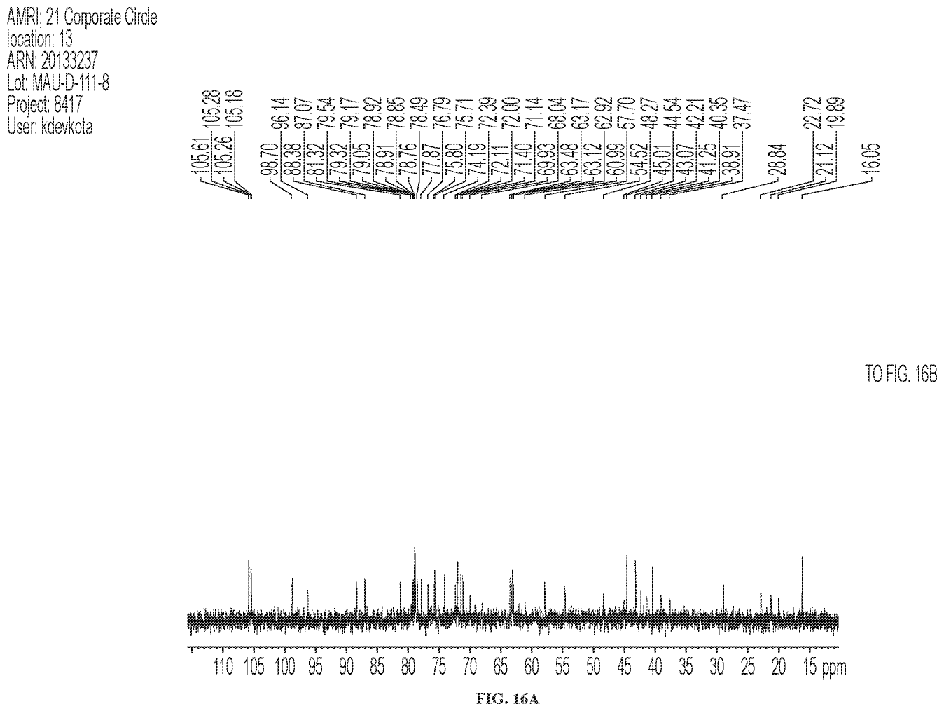

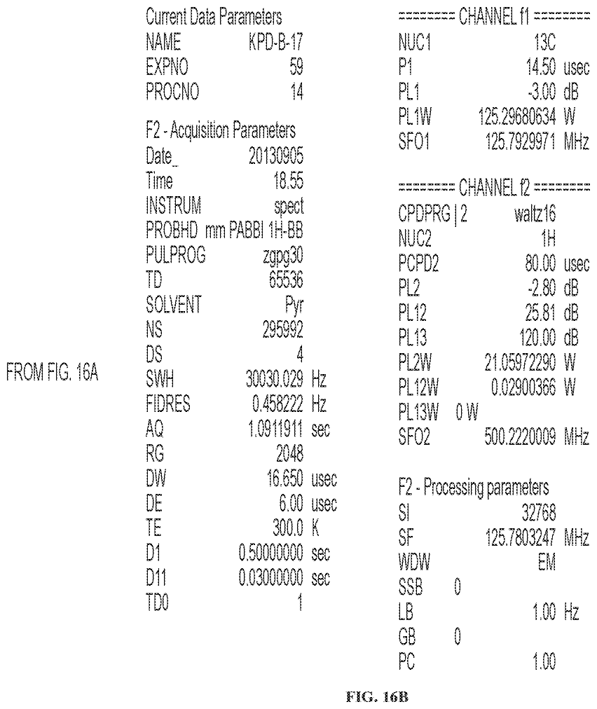

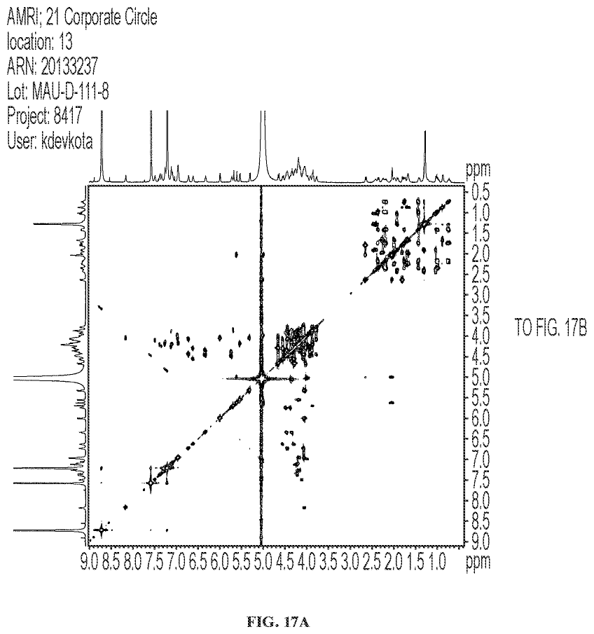

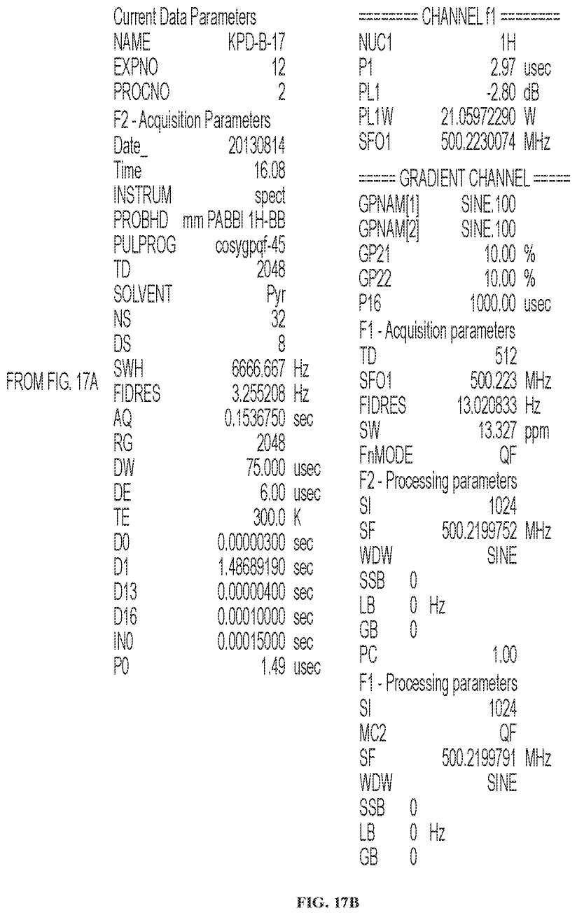

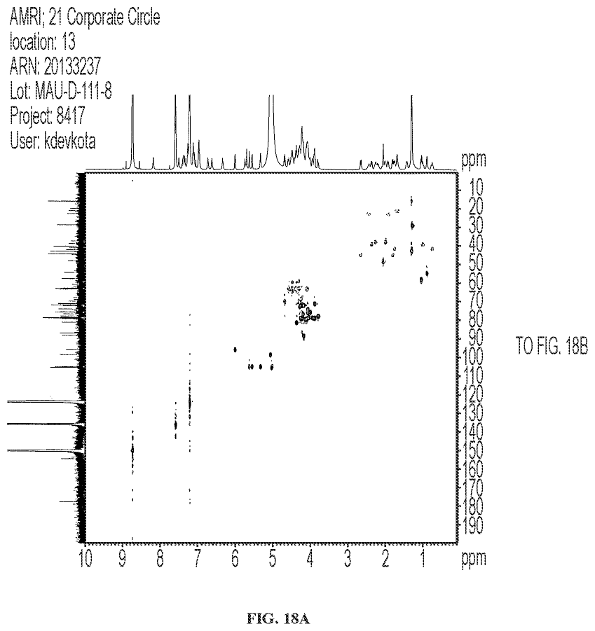

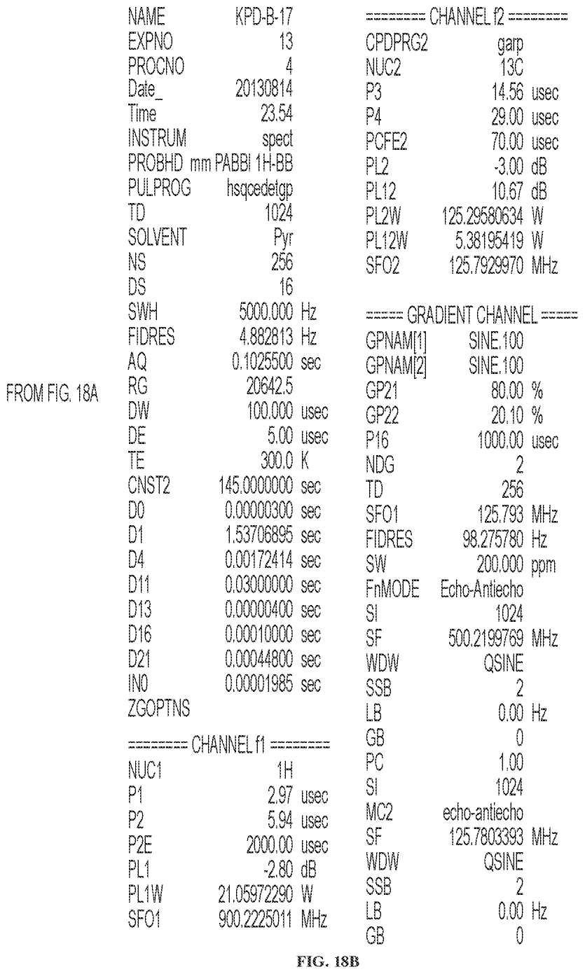

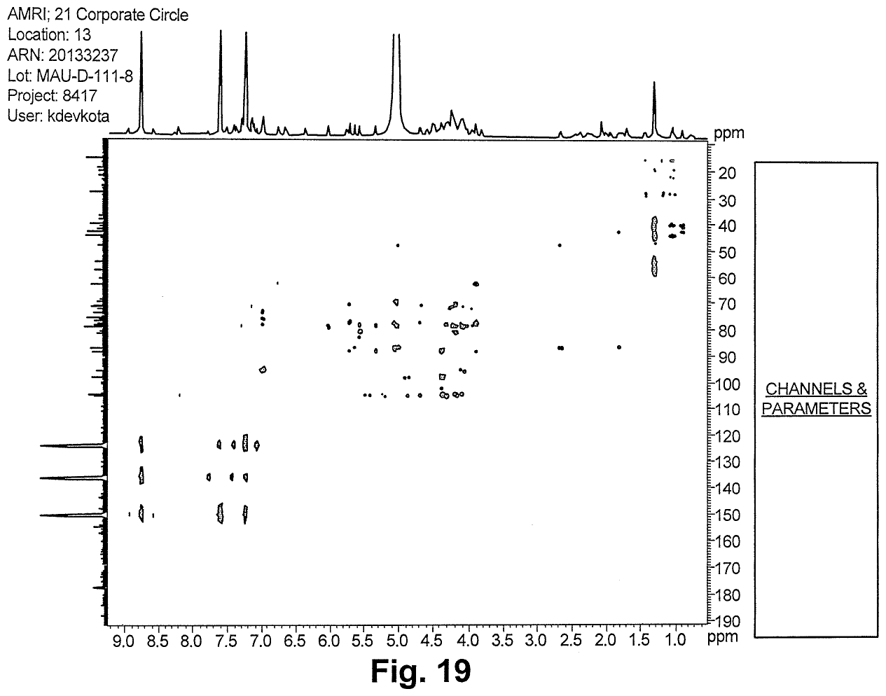

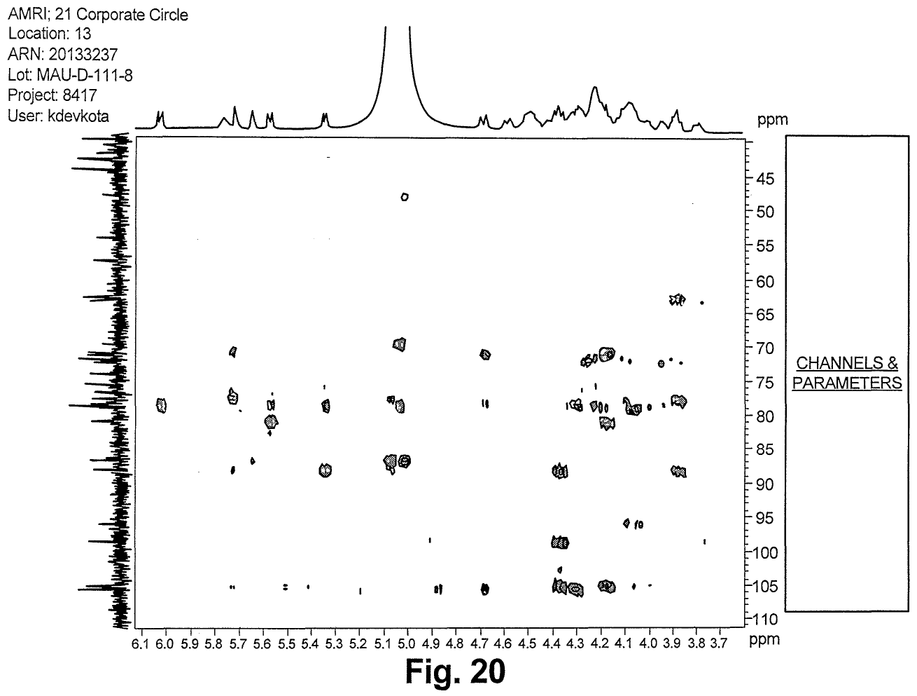

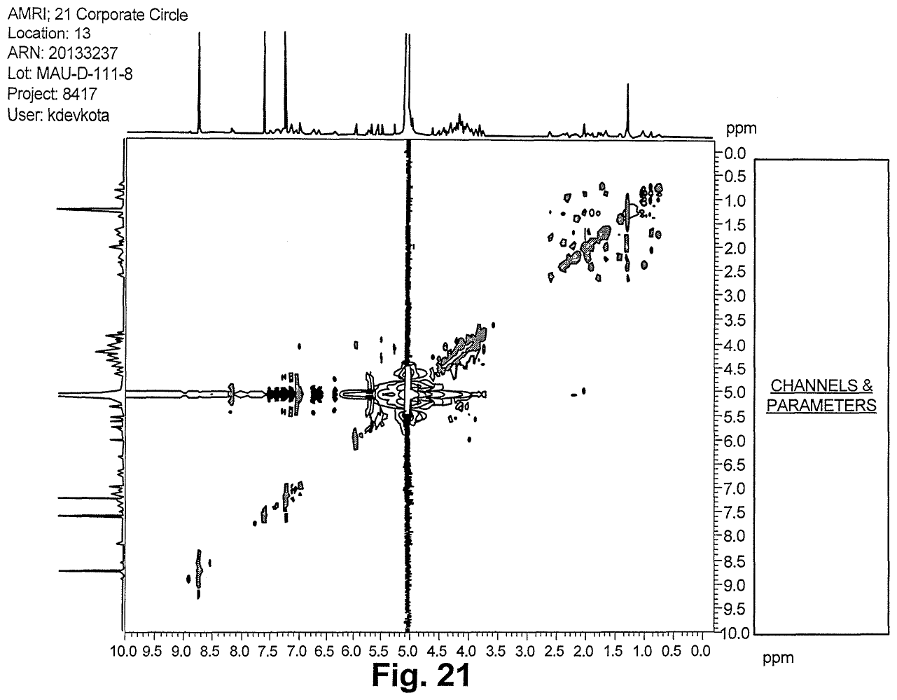

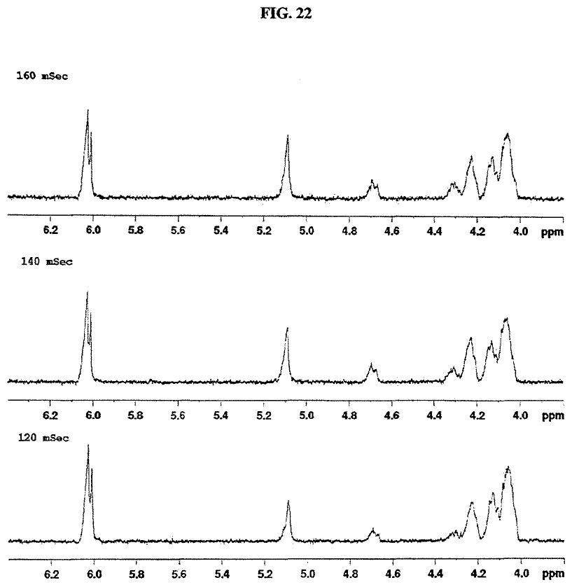

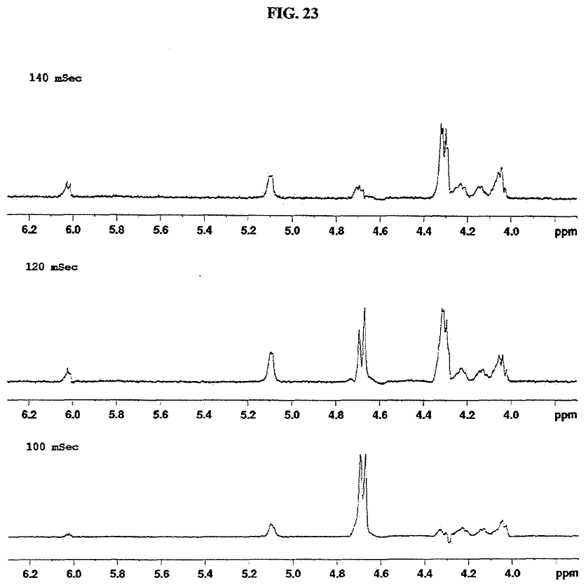

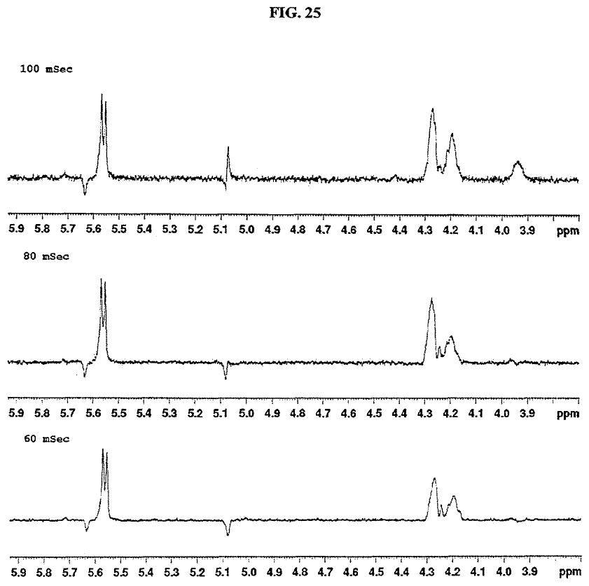

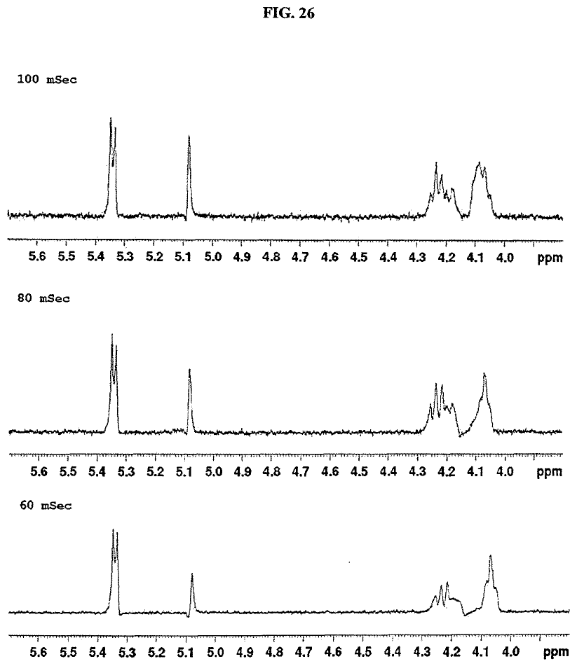

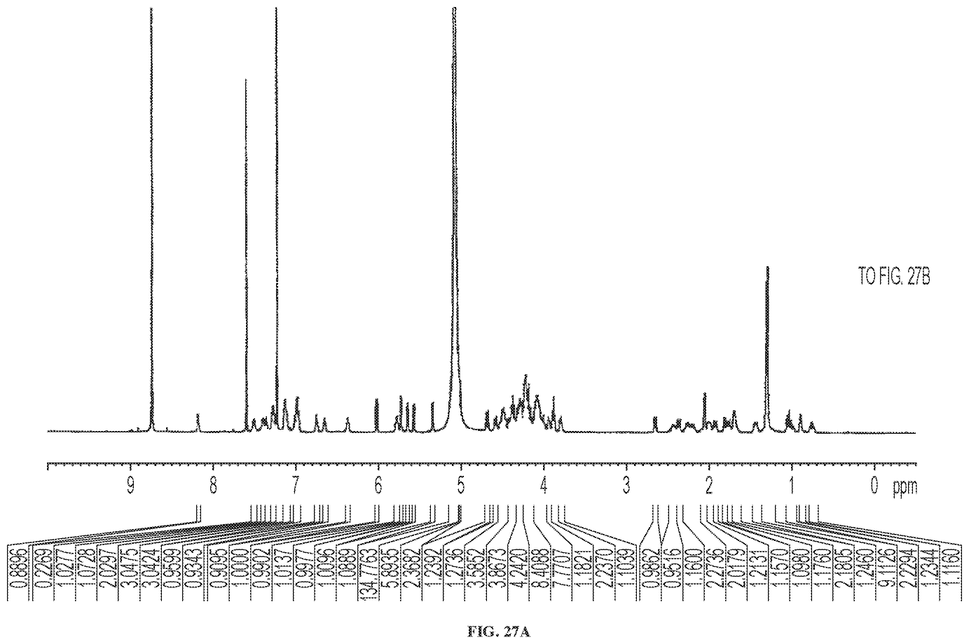

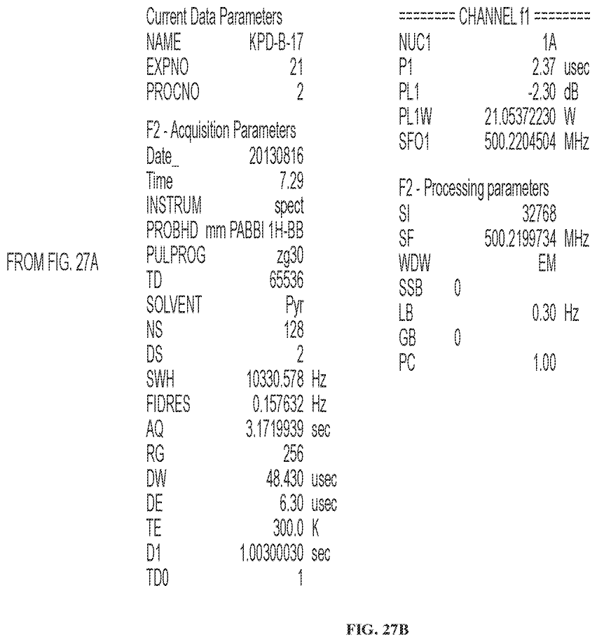

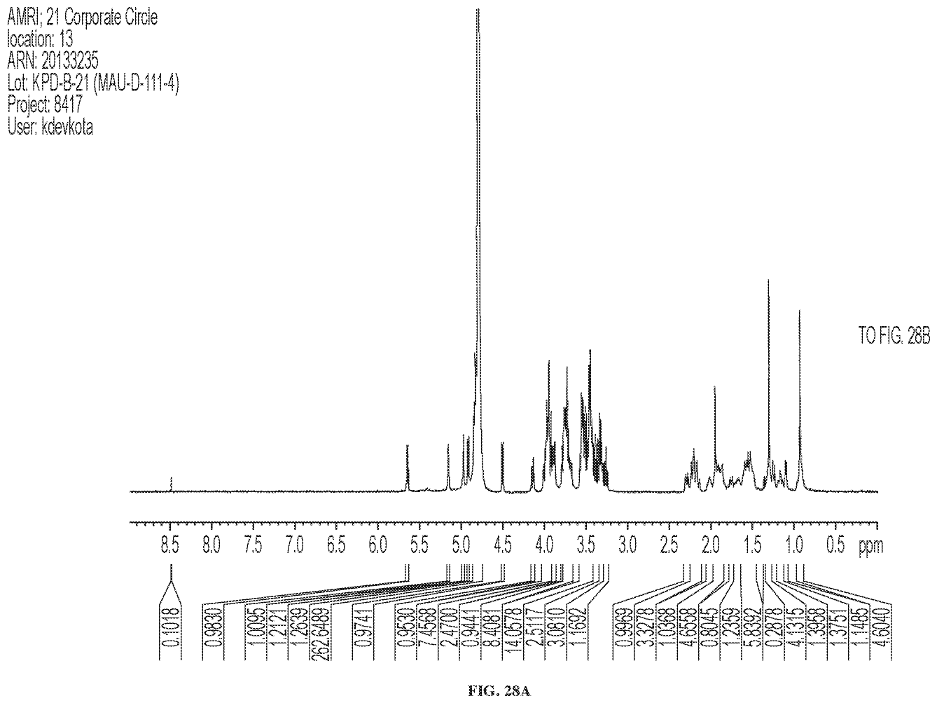

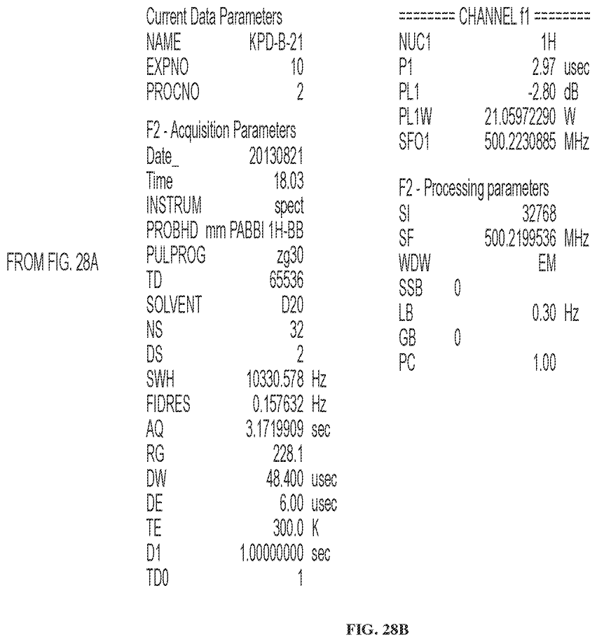

NMR Spectroscopy. A series of NMR experiments including ¹H NMR (FIG. 14), ¹³C NMR (FIGS. 15 and 16), ¹H-¹H COSY (FIG. 17), HSQC-DEPT (FIG. 18), HMBC (FIGS. 19 and 20), NOESY (FIG. 21) and 1D-TOCSY (FIG. 22-26) were performed to allow assignment of the compound. In the ¹H NMR acquired after −46 hrs of sample preparation (FIGS. 27-28), the anomeric resonance at δ_{H} 5.04 is resolved which was obscured by the solvent (HOD) in the original spectrum (FIG. 14)

The ¹H, ¹H-¹H COSY, ¹H-¹³C HSQC-DEPT and ¹H-¹³C HMBC NMR data indicated that the central core of the glycoside is a diterpene. The presence of five anomeric protons observed in the ¹H and ¹H-¹³C HSQC-DEPT spectra confirm five sugar units in the structure. The methylene ¹³C resonance at δ_{C} 69.9 in the ¹H-¹³C HSQC-DEPT spectrum indicated the presence of a 1→6 sugar linkage in the structure. The linkages of sugar units were assigned using ¹H-¹³C HMBC and 1D-TOCSY correlations.

A HMBC correlation from the methyl protons at δ_{H} 1.29 to the carbonyl at δ_{C} 177.7 allowed assignment of one of the tertiary methyl groups (C-18) as well as C-19 and provided a starting point for the assignment of the rest of the aglycone. Additional HMBC correlations from the methyl protons (H-18) to carbons at δ_{C} 38.9, 45.0, and 57.8 allowed assignment of C-3, C-4, and C-5. Analysis of the ¹H-¹³C HSQC-DEPT data indicated that the carbon at δ_{C} 38.9 was a methylene group and the carbon at δ_{C} 57.8 was a methine which were assigned as C-3 and C-5, respectively. This left the carbon at δ_{C} 45.0, which did not show a correlation in the HSQC-DEPT spectrum, to be assigned as the quaternary carbon, C-4. The ¹H chemical shifts for C-3 (δ_{H} 0.98 and 2j.36) and C-5 (δ_{H} 1.04) were assigned using the HSQC-DEPT data. A COSY correlation between one of the H-3 protons (δ_{H} 0.98) and a proton at δ_{H} 1.43 allowed assignment of one of the H-2 protons which in turn showed a correlation with a proton at δ_{H} 0.75 which was assigned to C-1. The remaining ¹H and ¹³C chemical shifts for C-1 and C-2 were then assigned on the basis of additional COSY and HSQC-DEPT correlations and are summarized in the table below.

¹H and ¹³C NMR (500 and 125 MHz,
pyridine-d₅), Assignments of Reb D2.

| | Reb D2 | |
|---|---|---|
| Position | ¹³C | ¹H |
| 1 | 41.3 | 0.75 t (11.0) |
| | | 1.76 m |
| 2 | 19.9 | 1.43 m |
| | | 2.20 m |
| 3 | 38.9 | 0.98 m |
| | | 2.36 d (12.1) |
| 4 | 45.0 | — |
| 5 | 57.8 | 1.04 d (12.5) |
| 6 | 22.7 | 1.92 m |
| | | 2.43 m |
| 7 | 42.2 | 1.22 m |
| | | 1.30 m |
| 8 | 43.1 | — |
| 9 | 54.5 | 0.88 brs |
| 10 | 40.3 | — |
| 11 | 21.1 | 1.65 m |
| | | 1.69 m |
| 12 | 37.5 | 1.99 m |
| | | 2.25 m |
| 13 | 87.1 | — |
| 14 | 44.5 | 1.80 d (11.7) |
| | | 2.65 d (11.7) |
| 15 | 48.3 | 1.31 m |
| | | 2.04 brs |
| 16 | 154.7 | — |
| 17 | 105.2 | 5.01 s |
| | | 5.64 s |
| 18 | 28.8 | 1.29 s |
| 19 | 177.7 | — |
| 20 | 16.0 | 1.30 s |

The other tertiary methyl singlet, observed at δ_{H} 1.30 showed HMBC correlations to C-1 and C-5 and was assigned as C-20. The methyl protons showed additional HMBC correlations to a quaternary carbon (δ_{C} 40.3) and a methine carbon (δ_{C} 54.5) which were assigned as C-10 and C-9, respectively. COSY correlations between H-5 (δ_{H} 1.04) and protons at δ_{H} 1.92 and 2.43 then allowed assignment of the H-6 protons which in turn showed correlations to protons at δ_{H} 1.22 and 1.30 which were assigned to C-7. The ¹³C chemical shifts for C-6 (δ_{C} 22.7) and C-7 (δ_{C} 42.2) were then determined from the HSQC-DEPT data. COSY correlations between H-9 (δ_{H} 0.88) and protons at δ_{H} 1.65 and 1.69 allowed assignment of the H-11 protons which in turn showed COSY correlations to protons at δ_{H} 1.99 and 2.25 which were assigned as the H-12 protons. The HSQC-DEPT data was then used to assign C-11 (δ_{C} 21.1) and C-12 (δ_{C} 37.5). HMBC correlations from the H-12 proton (δ_{H} 2.25) to carbons at δ_{C} 87.1 and 154.7 allowed assignment of C-13 and C-16, respectively. The olefinic protons observed at δ_{H} 5.01 and 5.64 showed HMBC correlations to C-13 and were assigned to C-17 (δ_{C} 105.2 via HSQC-DEPT). The olefinic protons H-17 and the methine proton H-9 showed HMBC correlations to a carbon at δ_{C} 48.3 which was assigned as C-15. An additional HMBC correlation from H-9 to a methylene carbon at δ_{C} 44.5 then allowed assignment of C-14. The ¹H chemical shifts at C-14 (δ_{H} 1.80 and 2.65) and C-15 (δ_{H} 1.31 and 2.04) were assigned using the HSQC-DEPT data.

Correlations observed in the NOESY spectrum were used to assign the relative stereochemistry of the central diterpene core. In the NOESY spectrum, NOE correlations were observed between H-14 and H-20 indicating that H-14 and H-20 are on the same face of the rings. Similarly, NOE correlations were observed between H-9 and H-5; H-9 and H-18 as well as H-5 and H-18 but NOE correlations were not observed between H-9 and H-14 indicating that H-5, H-9 and H-18 were on the opposite face of the rings compared to H-14 and H-20 as presented in FIG. 21. These data thus indicated that the relative stereochemistry in the central core was retained during the glycosylation step.

The key HMBC and COSY correlations used to assign the aglycone region are provided below:

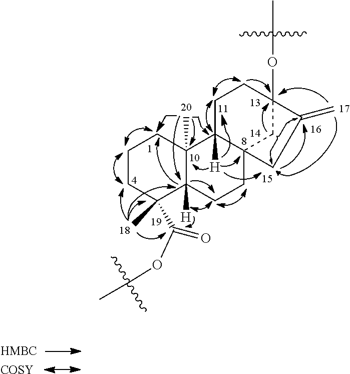

Analysis of the ¹H— ³C HSQC-DEPT data confirmed the presence of five anomeric protons. Three of the anomeric protons were well resolved at δ_{H} 6.02 (δ_{C} 96.1), 5.57 (Sc 105.3), and 5.34 (δ_{C} 105.3) in the ¹H NMR spectrum. The remaining two anomeric protons observed at δ_{H} 5.04 (δ_{C} 105.6) and 5.07 (δ_{C} 98.7) which were obscured by solvent (HOD) resonance in the ¹H spectrum were identified by ¹H-¹³C HSQC-DEPT data. The anomeric proton observed at δ_{H} 6.02 showed HMBC correlation to C-19 which indicated that it corresponds to the anomeric proton of Gl_{CI}. Similarly, the anomeric proton observed at δ_{H} 5.07 showed an HMBC correlation to C-13 allowing it to be assigned as the anomeric proton of Gl_{CII}.

The Gl_{CI} anomeric proton (δ_{H} 6.02) showed a COSY correlation to a proton at SH 4.07 was assigned as Gl_{CI} H-2 which in turn showed a COSY correlation to a proton at SH 4.22 (Gl_{CI} H-3) which showed a COSY correlation with a proton at δ_{H} 4.12 (Gl_{CI} H-4). Due to data overlap, the COSY spectrum did not allow assignment of H-5 or the H-6 protons. Therefore, a series of 1D-TOCSY experiments were performed using selective irradiation of the Gl_{CI} anomeric proton with several different mixing times. In addition to confirming the assignments for Gl_{CI} H-2 through H-4, the 1D-TOCSY data showed a proton at δ_{H} 4.04 assigned as Gl_{CI} H-5 and a proton at δ_{H} 4.68 assigned as one of the Gl_{CI} H-6 protons. The latter proton was also used for 1D-TOCSY experiments. The selective irradiation of H-6 with several different mixing times also confirmed the assignment of Gl_{CI} H-1 to H-5 as well as the remaining methylene proton of H-6 (δ_{H} 4.30). Assignment of the ¹³C chemical shifts for Gl_{CI} C-2 (δ_{C} 74.2), C-3 (δ_{C} 79.1), C-4 (δ_{C} 72.1), C-5 (δ_{C} 78.5), and C-6 (δ_{C} 69.9) was determined using the ¹H-¹³C HSQC-DEPT data to complete the assignment of Gl_{CI}. Furthermore, the presence of a methylene ¹³C resonance at δ_{C} 69.9 in the ¹H-¹³C HSQC-DEPT spectrum indicated a 1→6 sugar linkage of Gl_{CI} in the structure.

Out of four remaining unassigned glucose moieties, one was assigned as a substituent at C-6 of Gl_{CI} on the basis of ¹H-¹³C HSQC-DEPT, HMBC, and 1D-TOCSY correlations. The relatively downfield shift of a methylene ¹³C resonance of Gl_{CI} at δ_{C} 69.9 in the HSQC-DEPT spectrum indicated a 1→6 sugar linkage of Gl_{CI}. The anomeric proton observed at δ_{H} 5.04 showed HMBC correlation to Gl_{CI} C-6 and was assigned as the anomeric proton of Gl_{CV}. Similarly, methylene protons of Gl_{CI} showed HMBC correlations to anomeric carbon of Gl_{CV} confirming the presence of a 1→6 sugar linkage between Gl_{CI} and Gl_{CV}. The Gl_{CV} anomeric proton showed a COSY correlation to a proton at δ_{H} 4.00 which was assigned as Gl_{CV} H-2 which in turn showed a COSY correlation to a proton at δ_{H} 4.22 (Gl_{CV} H-3). Due to data overlap, the COSY spectrum did not allow assignment of Gl_{CV} H-4 based on the COSY correlation of Gl_{CV} H-3. However, in the HMBC spectrum, Gl_{CV} H-3 showed a correlation to Gl_{CV} C-5 (δ_{C} 78.9). In HSQC-DEPT spectrum, Gl_{CV} C-5 showed a correlation to δ_{H} 3.89 (Gl_{CV} H-5). The Gl_{CV} H-5 showed COSY correlations to δ_{H} 4.21, 4.37, and 4.48. In the HSQC-DEPT spectrum, δ_{H} 4.21 showed a correlation to δ_{c} 71.4 (Gl_{CV} H-4), while δ_{H} 4.37 and 4.48 showed a correlation to δ_{c} 63.1 and were assigned to Gl_{CV} H-6a and H-6b, respectively. Assignment of the ¹³C chemical shifts for Gl_{CV} C-2 (δ_{C} 75.7) and C-3 (δ_{C} 79.1) was determined using the ¹H-¹³C HSQC-DEPT data to complete the assignment of Gl_{CV}.

A summary of the ¹H and ¹³C chemical shifts for the glycoside at C-19 are shown in the following table:

¹H and ¹³C NMR (500 and 125 MHz,
pyridine-d₅), Assignments of the reb D2 C-19 glycoside.

| | Reb D2 | |
|---|---|---|
| Position | ¹³C | ¹H |
| Glc_{I}-1 | 96.1 | 6.02 d (8.1) |
| Glc_{I}-2 | 74.2 | 4.07 m |
| Glc_{I}-3 | 79.1^{#} | 4.22 m^{#} |
| Glc_{I}-4 | 72.1 | 4.12 m |
| Glc_{I}-5 | 78.5 | 4.04 m |
| Glc_{I}-6 | 69.9 | 4.30 m |
| | | 4.68 d (10.7) |
| Glc_{V}-1 | 105.6 | 5.04 (8.1) |
| Glc_{V}-2 | 75.7 | 4.00 m |
| Glc_{V}-3 | 79.1^{#} | 4.22 m^{#} |
| Glc_{V}-4 | 71.4 | 4.21 m |
| Glc_{V}-5 | 78.9 | 3.89 m |
| Glc_{V}-6 | 63.1 | 4.37 m |
| | | 4.48 m |

^{#1}H and ¹³C values can be exchangeable between positions Glc₁-3, Glc_{V}-3 and Glc_{IV}-3.

A summary of the key HMBC, COSY, and 1D-TOCSY correlations used to assign the C-19 glycoside region are provided below.

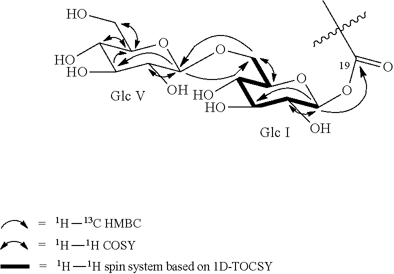

Assignment of Gl_{CII} was carried out in a similar manner. The Gl_{CII} anomeric proton (δ_{H} 5.07) showed a COSY correlation to a proton at δ_{H} 4.37, assigned as Gl_{CII} H-2, which in turn showed a COSY correlation to a proton at δ_{H} 4.18 (Gl_{CII} H-3). This latter proton showed an additional correlation with a proton at δ_{H} 3.88 (Gl_{CII} H-4) which also showed a COSY correlation to a proton at δ_{H} 3.79 (Gl_{CII} H-5). Gl_{CII} H-5 also showed a COSY correlation to Gl_{CII} H-6 protons (δ_{H} 4.08 and 4.46). Assignment of the ¹³C chemical shifts for Gl_{CII} C-2 (δ_{C} 81.3), C-3 (δ_{C} 88.4), C-4 (δ_{C} 71.1), C-5 (δ_{C} 77.9), and C-6 (δ_{C} 63.2) was determined using the HSQC-DEPT data. HMBC correlations from Gl_{CII} H-3 to C-2 and C-4 and also from Gl_{CII} H-4 to C-2 and C-5 confirmed the assignments made above. Additional HMBC correlations of Gl_{CII} H-4 to Gl_{CII} C-6 further support to complete the assignment of Gl_{CII}.

Two of the remaining unassigned glucose moieties were assigned as substituents at C-2 and C-3 of Gl_{CII} on the basis of HMBC correlations. The anomeric proton observed at δ_{H} 5.57 showed a HMBC correlation to Gl_{CII} C-2 and was assigned as the anomeric proton of Gl_{CIII}. The anomeric proton observed at δ_{H} 5.34 showed a HMBC correlation to Gl_{CII} C-3 and was assigned as the anomeric proton of Gl_{CIV}. The reciprocal HMBC correlations from Gl_{CII} H-2 to the anomeric carbon of Gl_{CII} and from Gl_{CII} H-3 to the anomeric carbon of Gl_{CIV} were also observed.

The anomeric proton of Gl_{CII} (δ_{H} 5.57) showed a COSY correlation with a proton at δ_{H} 4.19 which was assigned as Gl_{CII} H-2. Due to data overlap, the COSY spectrum did not allow assignment of H-3 to H-6 protons. Therefore, a series of 1D-TOCSY experiments were performed using selective irradiation of the Gl_{CII} anomeric proton with several different mixing times. In addition to confirming the assignments for Gl_{CII} H-2, the 1D-TOCSY data showed protons at δ_{H} 4.24 (Gl_{CII} H-3), δ_{H} 4.27 (Gl_{CII} H-4), and SH 3.94 (Gl_{CII} H-5). Once H-4 was assigned using 1D-TOCSY data, COSY correlations from H-4 to H-5 and in turn to H-6 were used to assign H-6. In the COSY spectrum, Gl_{CII} H-4 showed a correlation to Gl_{CII} H-5, which in turn showed COSY correlations to δ_{H} 4.41 and 4.50 of Gl_{CII} H-6a and H-6b, respectively. The ¹³C chemical shifts for Gl_{CII} C-2 (δ_{C} 76.8), C-3 (6c 78.9), C-4 (6c 72.4), C-5 (6c 78.8), and C-6 (6c 63.5) were then determined using the ¹H-¹³C HSQC-DEPT correlations to complete the assignment of Gl_{CII}.

The anomeric proton of Gl_{CIV} (δ_{H} 5.34) showed a COSY correlation with a proton at δ_{H} 4.06 which was assigned as Gl_{CIV} H-2. Due to data overlap, the COSY spectrum did not allow assignment of H-3 to H-6 protons. Therefore, a series of 1D-TOCSY experiments were performed using selective irradiation of the Gl_{CIV} anomeric proton with several different mixing times. In addition to confirming the assignments for Gl_{CIV} H-2, the 1D-TOCSY data showed protons at δ_{H} 4.22 (Gl_{CIV} H-3), δ_{H} 4.18 (Gl_{CIV} H-4), and SH 4.10 (Gl_{CIV} H-5). Once H-4 was assigned using 1D-TOCSY data, COSY correlations from H-4 to H-5 and in turn to H-6 were used to assign H-6. In the COSY spectrum, Gl_{CIV} H-4 showed a correlation to Gl_{CIV} H-5, which in turn showed COSY correlations to δ_{H} 4.32 and 4.58, Gl_{CIV} H-6a and H-6b, respectively. The ¹³C chemical shifts for Gl_{CIV} C-2 (6c 75.8), C-3 (δ_{C} 78.9), C-4 (δ_{C} 72.0), C-5 (δ_{C} 79.3), and C-6 (δ_{C} 62.9) were then determined using the ¹H-¹³C HSQC-DEPT correlations to complete the assignment of Gl_{CIV}.

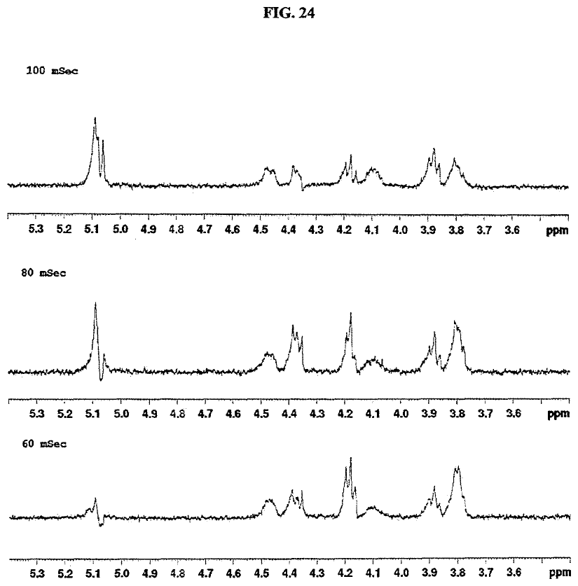

The large coupling constants observed for the anomeric protons of the glucose moieties at δ_{H} 6.02 (d, J=8.1 Hz), 5.57 (d, J=7.6 Hz), 5.34 (d, J=7.9 Hz) and δ_{H} 5.04 (d, J=8.1 Hz), suggested their β-orientation (FIGS. 14, 27, and 28). While the remaining anomeric proton at δ_{H} 5.07 was obscured by the solvent resonance (HDO) it's coupling constant (J=˜8 Hz) evident from 1D TOCSY data (FIG. 24) also indicated β-orientation.

A summary of the ¹H and ¹³C chemical shifts for the glycoside at C-13 are shown in the table below:

¹H and ¹³C NMR (500 and 125 MHz,
pyridine-d₅), Assignments of the Reb D2 C-13 glycoside.

| | Reb D2 | |
|---|---|---|
| Position | ¹³C | ¹H |
| Glc_{II}-1 | 98.7 | 5.07 (~8)* |
| Glc_{II}-2 | 81.3 | 4.37 m |
| Glc_{II}-3 | 88.4 | 4.18 m |
| Glc_{II}-4 | 71.1 | 3.88 m |
| Glc_{II}-5 | 77.9 | 3.79 m |
| Glc_{II}-6 | 63.2 | 4.08 m |
| | | 4.47 m |
| Glc_{III}-1 | 105.3 | 5.57 d (7.6) |
| Glc_{III}-2 | 76.8 | 4.19 m |
| Glc_{III}-3 | 78.9 | 4.24 m |
| Glc_{III}-4 | 72.4 | 4.27 m |
| Glc_{III}-5 | 78.8 | 3.94 m |
| Glc_{III}-6 | 63.5 | 4.41 m |
| | | 4.50 m |
| Glc_{IV}-1 | 105.3 | 5.34 d (7.9) |
| Glc_{IV}-2 | 75.8 | 4.06 m |
| Glc_{IV}-3 | 78.9^{#} | 4.22 m^{#} |
| Glc_{IV}-4 | 72.0 | 4.18 m |
| Glc_{IV}-5 | 79.3 | 4.10 m |
| Glc_{IV}-6 | 62.9 | 4.32 m |
| | | 4.58 m |

*Anomeric proton was obscured by solvent (HDO) resonance, coupling constant value obtained from 1D-TOCSY data.
^{#1}H and ¹³C values can be exchangeable between Glc₁-3, Glc_{V}-3 and Glc_{IV}-3.

A summary of the key HMBC, COSY, and 1D-TOCSY correlations used to assign the C-13 glycoside region are provided below:

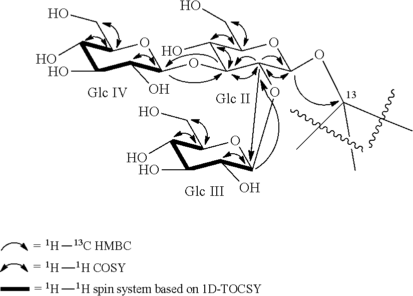

NMR and MS analyses allowed a full assignment of structure, shown below. The chemical name of the compound is 13-[(2-O-β-D-glucopyranosyl-3-O-β-D-glucopyranosyl-β-D-glucopyranosyl)oxy]ent-kaur-16-en-19-oic acid-[(6-O-β-D-glucopyranosyl-β-D-glucopyranosyl) ester](rebaudioside D2 or reb D2). The compound is an isomer of rebaudioside D.

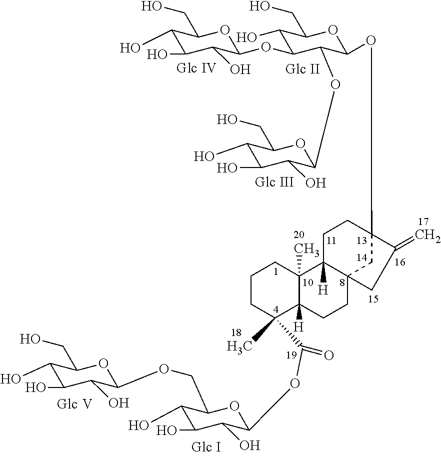

### Example 40

Isolation and Characterization of Reb M2 Crude Reaction Sample. The sample, Lot CB-2977-106, used for isolation was prepared

according to Example 22 with UGTSL (GI #460409128).

HPLC Analysis. Preliminary HPLC analyses was performed using a Waters 2695 Alliance System with the following method: Phenomenex Synergi Hydro-RP, 4.6×250 mm, 4 μm (p/n 00G-4375-E0); Column Temp: 55° C.; Mobile Phase A: 0.0284% NH₄OAc and 0.0116% HOAc in water; Mobile Phase B: Acetonitrile (MeCN); Flow Rate: 1.0 mL/min; Injection volume: 10 μL. Detection was by UV (210 nm) and CAD.

Gradient:

| Time (min) | % A | % B |
|---|---|---|
| 0.0-5.0 | 100 | 0 |
| 20 | 20 | 80 |
| 25 | 20 | 80 |
| 30 | 100 | 0 |

Analyses of semi-preparative purification fractions were performed with the following method: Waters Atlantis dC18, 4.6×100 mm, 5 μm (p/n 186001340); Mobile Phase A: 25% MeCN in water; Mobile Phase B: 30% MeCN in water; Flow Rate: 1.0 mL/min; Injection volume: 10 μL. Detection was by CAD.

Gradient:

| Time (min) | % A | % B |
|---|---|---|
| 0.0-8.5 | 75 | 25 |
| 10.0 | 71 | 29 |
| 16.5 | 70 | 30 |
| 18.5-24.5 | 66 | 34 |
| 26.5-29.0 | 48 | 52 |
| 31-37 | 30 | 70 |
| 38 | 75 | 25 |

LC-MS. Preliminary analysis of the semi-synthetic steviol glycoside mixture was carried out on a Waters AutoPurification HPLC/MS System with a Waters 3100 Mass Detector operating in negative ion mode. Analysis of the sample was performed using the following method: Phenomenex Synergi Hydro-RP, 4.6×250 mm, 4 μm (p/n 00G-4375-E0); Column Temp: 55° C.; Mobile Phase A: 0.0284% NH₄OAc and 0.0116% HOAc in water; Mobile Phase B: MeCN; Flow Rate: 1.0 mL/min; Injection volume: 10 μL. Detection was by UV (210 nm), and MSD (−ESI m/z 500-2000). Gradient conditions were as listed above.

Isolation by HPLC. The purification was performed in two steps. The first method used for the semi-preparative purification is summarized below. Column: Waters Atlantis dC18, 30×100 mm, 5 μm (p/n 186001375); Mobile Phase A: 25% MeCN in water; Mobile Phase B: 30% MeCN in water; Flow Rate: 45 mL/min; Injection load: 160 mg dissolved in 20 mL of water. Detection was by UV (205 nm).

Gradient:

| Time (min) | % A | % B |
|---|---|---|
| 0.0-5.0 | 100 | 0 |
| 20 | 20 | 80 |
| 25 | 20 | 80 |
| 30 | 100 | 0 |

The secondary purification used the same column and conditions, but isocratic mobile phase: 20% MeCN in water.

MS and MS/MS. MS and MS/MS data were generated with a Waters QTof Premier mass spectrometer equipped with an electrospray ionization source. Samples were analyzed by negative ESI. Samples were diluted with H₂O:MeCN (1:1) by 50 fold and introduced via infusion using the onboard syringe pump. The samples were diluted to yield good s/n which occurred at an approximate concentration of 0.01 mg/mL. NMR. The sample was prepared by dissolving −1.0 mg in 150 μL of D20 and NMR data were acquired on a Bruker Avance 500 MHz instrument with a 2.5 mm inverse detection probe. The ¹H NMR and ¹³C NMR spectra were referenced to the residual solvent signal HDO (δ_{H} 4.79 ppm) and TSP (6c 0.00 ppm), respectively.

Results and Discussion

Isolation and Purification. Isolation was performed using on a steviol glycoside mixture, Lot number CB-2977-106, prepared according to Example 22 with UGTSL (GI #460409128). The material was analyzed by LC-MS using the method described above (FIG. 11). The targeted peak of interest was that at 4.1 min in the TIC chromatogram. The mass spectrum of this peak provided a [M-H]⁻ ion at m z 1289.7. The provided sample was preliminarily processed in a single injection (160 mg) using the first method condition provided above. This method fractionated the material into ‘polar’ and ‘non-polar’ mixtures of glycosides. The ‘polar’ mixture was then reprocessed using the second-step conditions provided above. The semi-preparative HPLC trace is shown in FIG. 12. From this semi-preparative collection, the peak was isolated with a purity >99% (CAD, AUC). The fraction analysis is provided in FIG. 13. Following the purification, the fractions were concentrated by rotary evaporation at 35° C. and lyophilized. Approximately 1 mg was obtained.

Mass Spectrometry. The ESI- TOF mass spectrum acquired by infusing a sample of CC-00300 showed a [M-H]⁻ ion at m z 1289.5266. The mass of the [M-H]⁻ ion was in good agreement with the molecular formula C₅₆H₉₀O₃₃ (calcd for C₅₆H₈₉O₃₃: 1289.5286, error: −1.6 ppm) expected for reb M2. The MS data confirmed that CC-00300 has a nominal mass of 1290 Daltons with the molecular formula, C₅₆H₉₀O₃₃.

The MS/MS spectrum (selecting the [M-H]⁻ ion at m z 1289.5 for fragmentation) indicated the loss of three glucose units at m/z 803.3688 and sequential loss of three glucose moieties at m z 641.3165, 479.2633 and 317.2082.

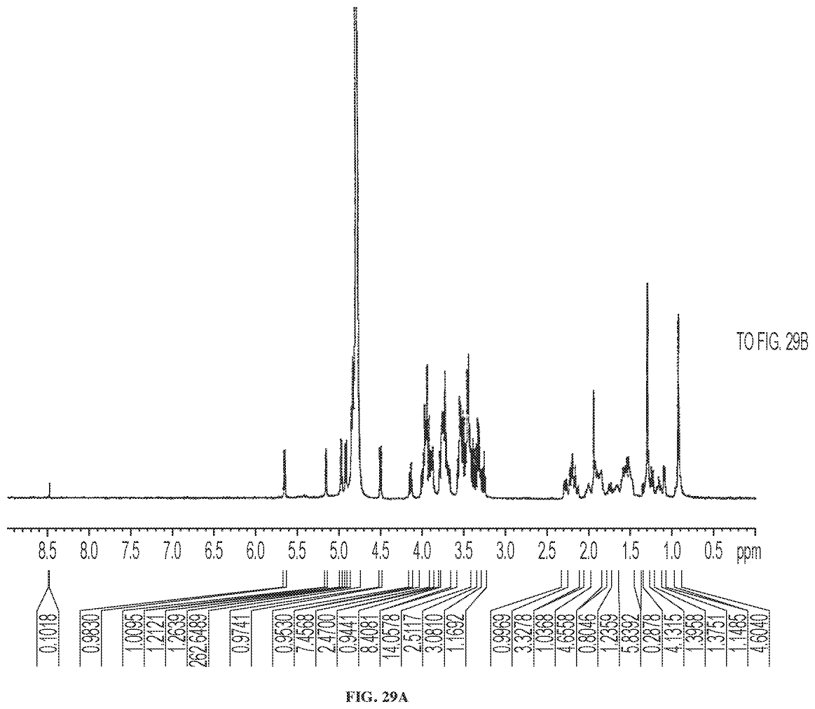

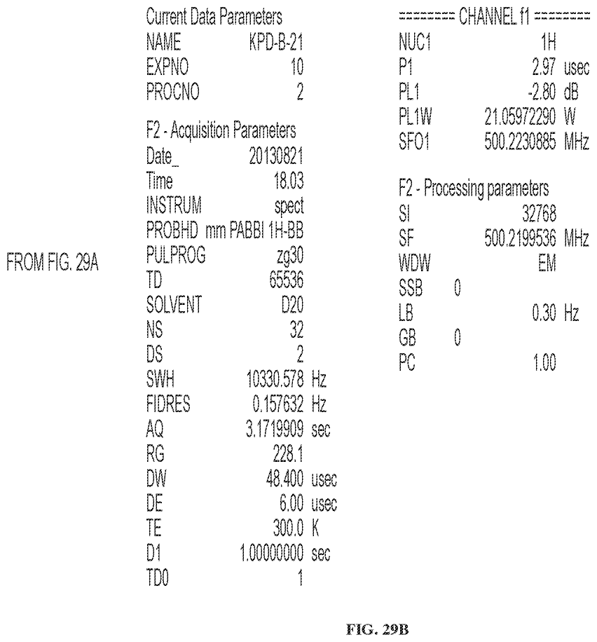

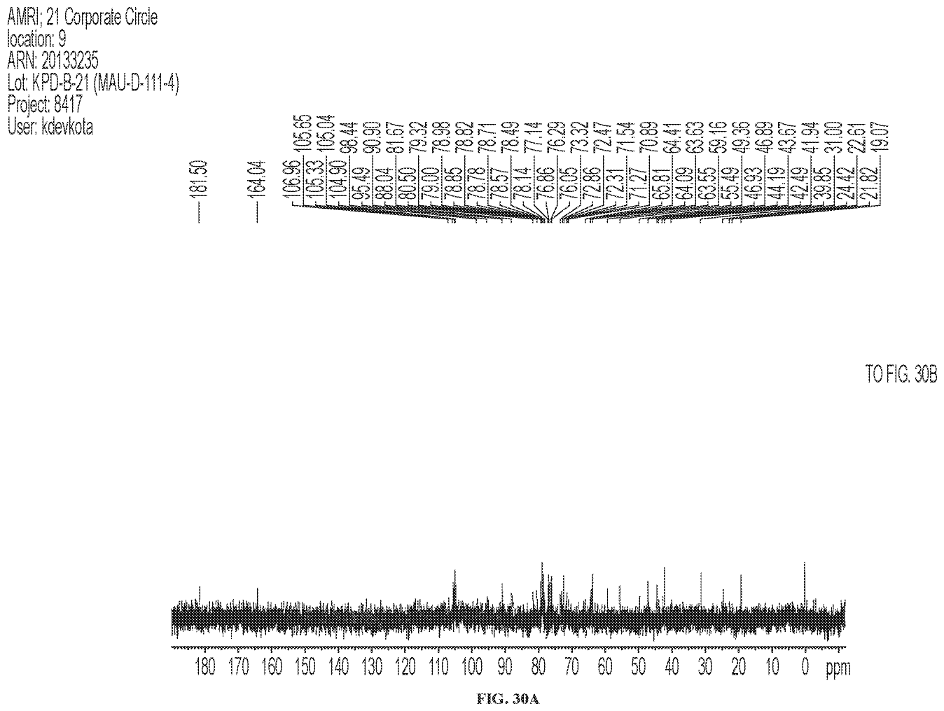

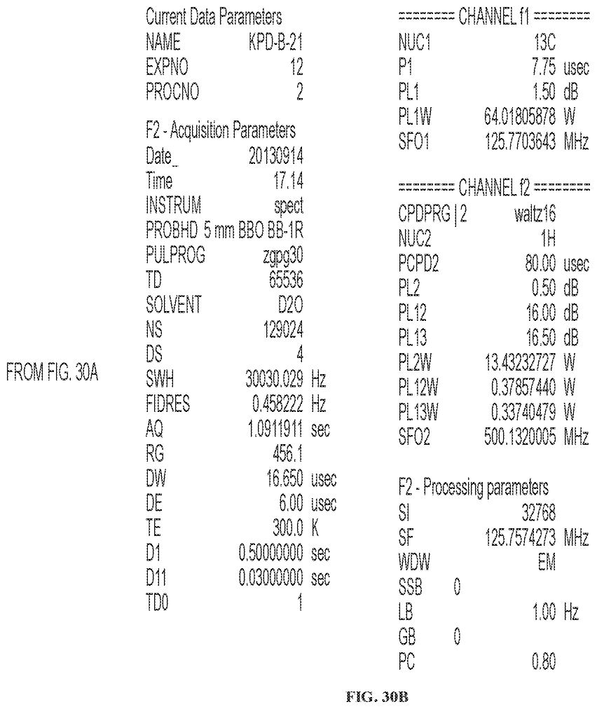

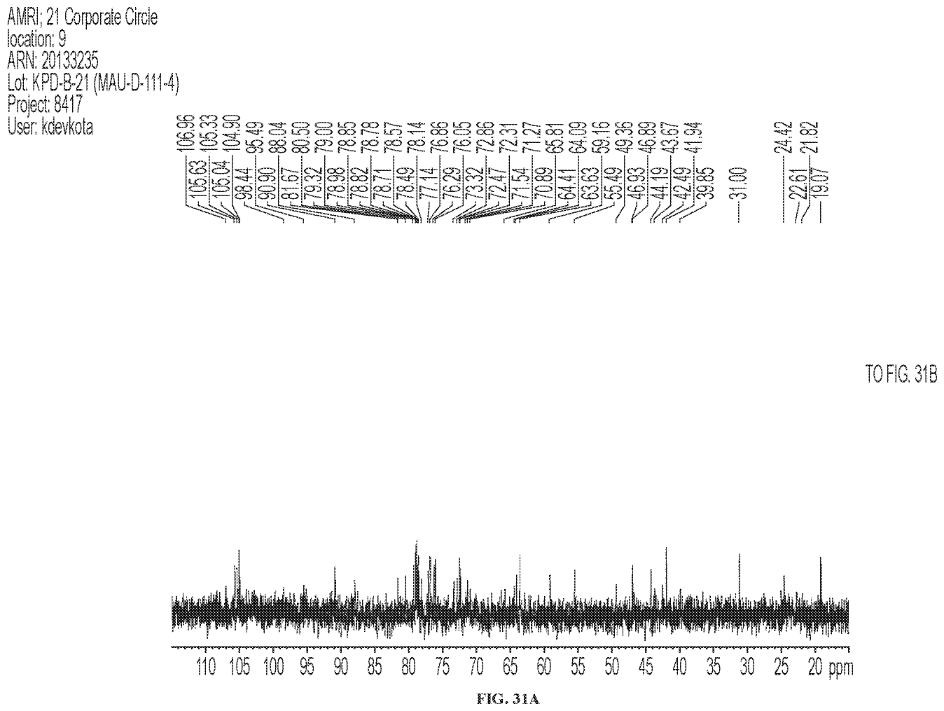

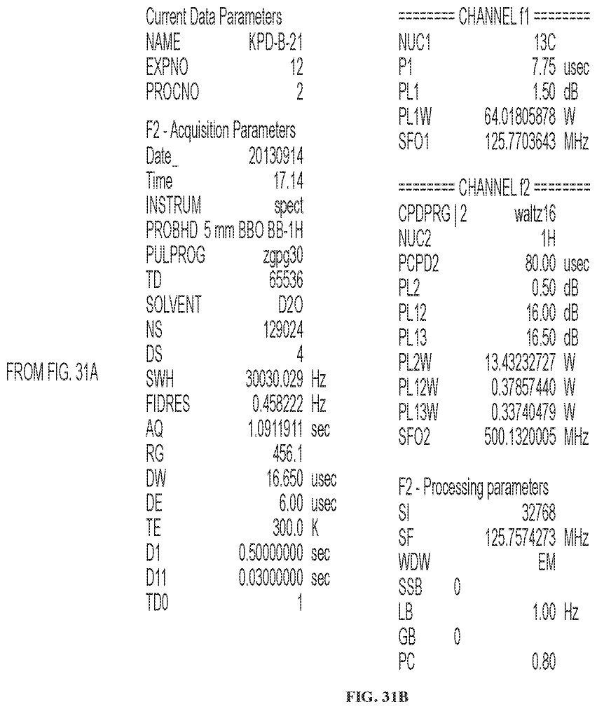

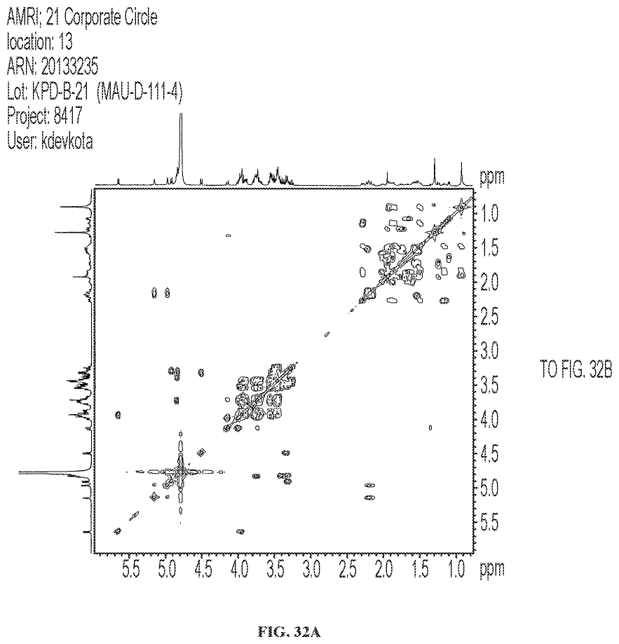

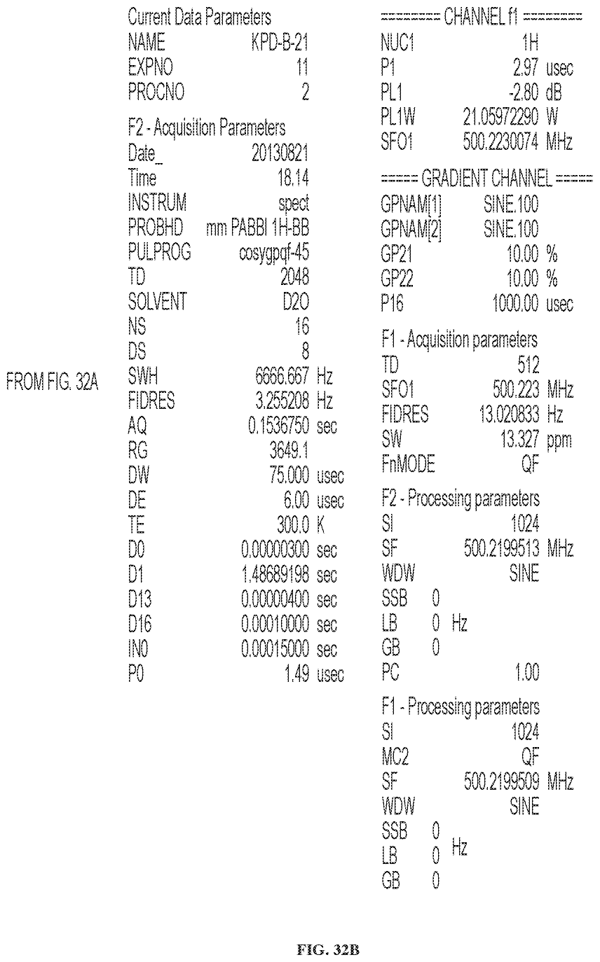

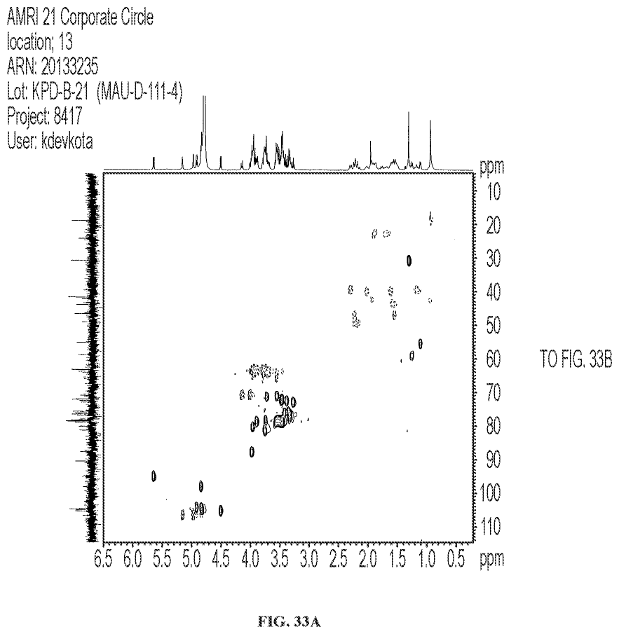

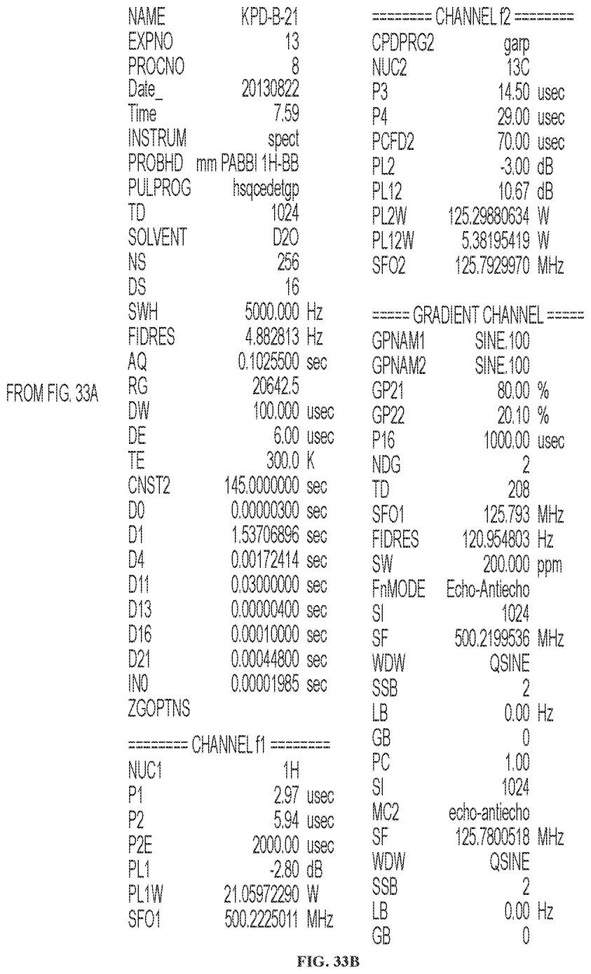

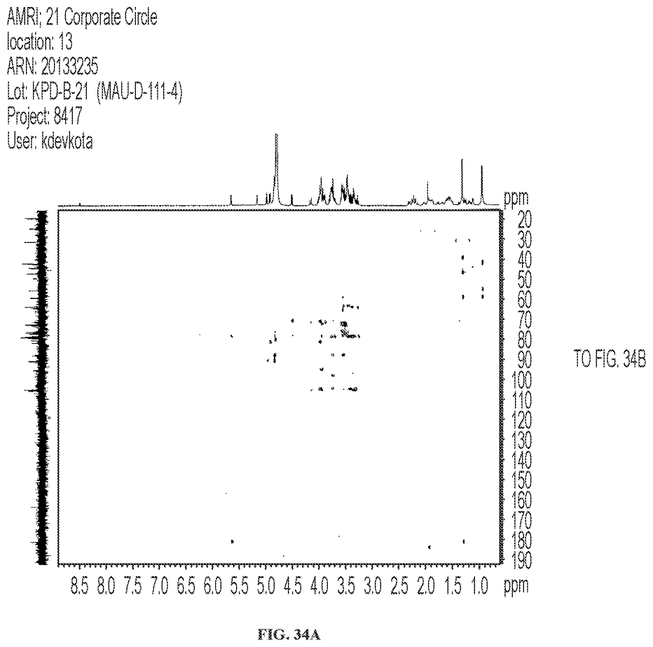

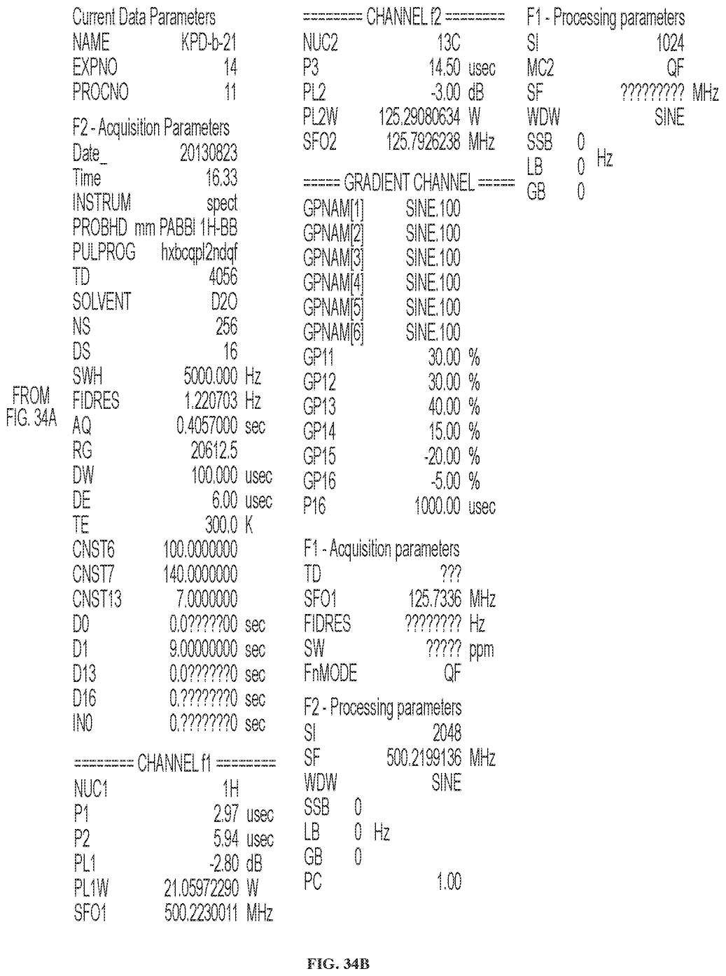

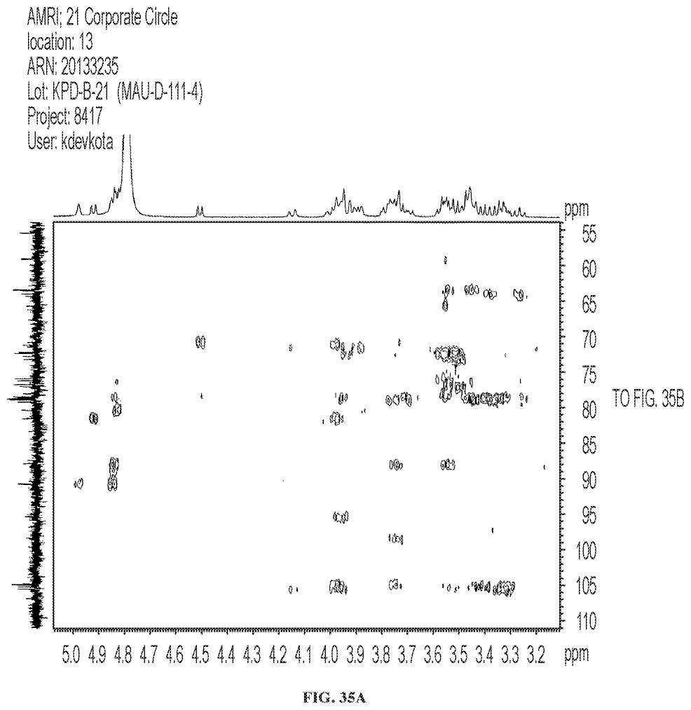

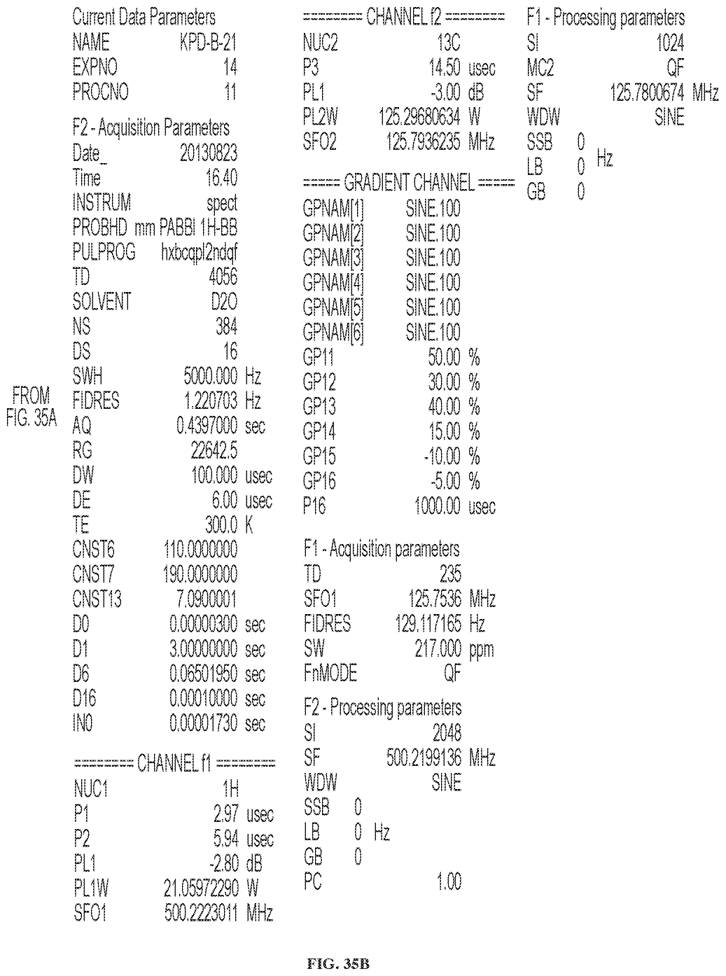

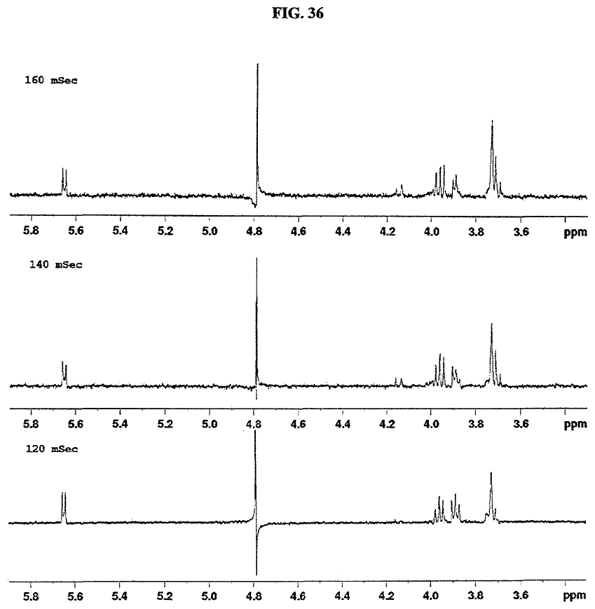

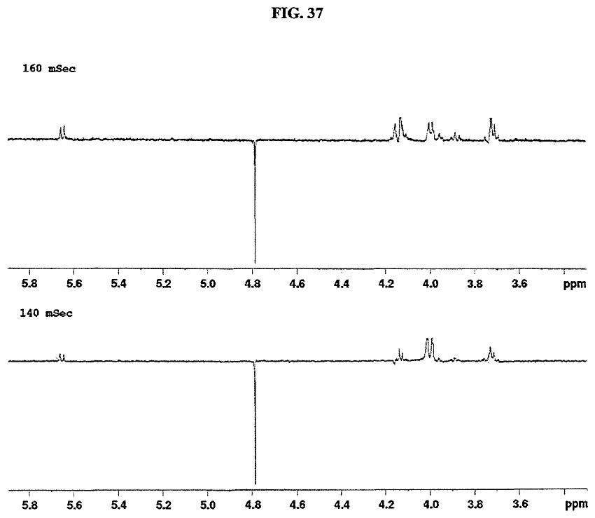

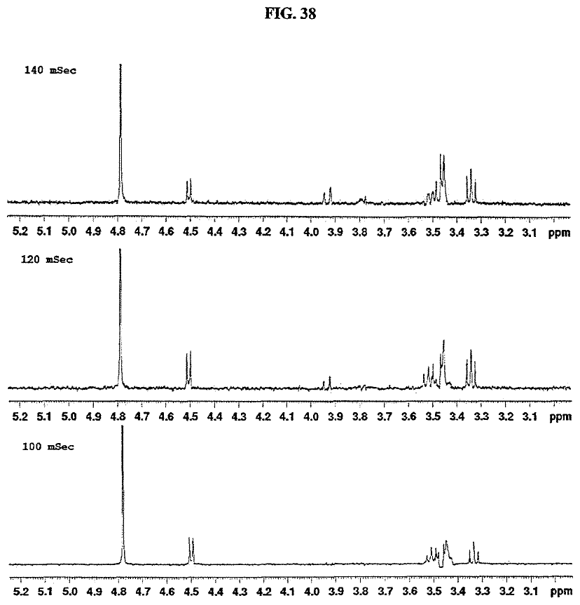

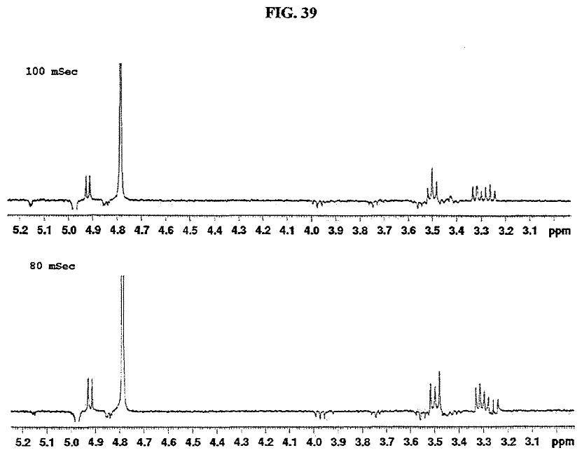

NMR Spectroscopy. A series of NMR experiments including ¹H NMR (FIG. 29), ¹³C NMR (FIGS. 30 and 31), ¹H-¹H COSY (FIG. 32), HSQC-DEPT (FIG. 33), HMBC (FIGS. 34 and 35), and 1D-TOCSY (FIG. 36-39) were performed to allow assignment of reb M2.

The ¹H, ¹H-¹H COSY, ¹H-¹³C HSQC-DEPT and ¹H-¹³C HMBC NMR data indicated that the central core of the glycoside is a diterpene. The presence of six anomeric protons observed in the ¹H and ¹H-¹³C HSQC-DEPT spectra confirm six sugar units in the structure. The methylene ¹³C resonance at δ_{C} 70.9 in the ¹H-¹³C HSQC-DEPT spectrum indicated the presence of a 1→6 sugar linkage in the structure. The linkages of sugar units were assigned using ¹H-¹³C HMBC and 1D-TOCSY correlations.

A HMBC correlation from the methyl protons at δ_{H} 1.29 to the carbonyl at δ_{C} 181.5 allowed assignment of one of the tertiary methyl groups (C-18) as well as C-19 and provided a starting point for the assignment of the rest of the aglycone. Additional HMBC correlations from the methyl protons (H-18) to carbons at δ_{C} 39.8, 43.7, and 59.2 allowed assignment of C3, C4, and C5. Analysis of the ¹H-¹³C HSQC-DEPT data indicated that the carbon at δ_{C} 39.8 was a methylene group and the carbon at δ_{C} 59.2 was a methine which were assigned as C-3 and C-5, respectively. This left the carbon at δ_{C} 43.7, which did not show a correlation in the HSQC-DEPT spectrum, to be assigned as the quaternary carbon, C-4. The ¹H chemical shifts for C-3 (δ_{H} 1.16 and 2.28) and C-5 (δ_{H} 1.24) were assigned using the HSQC-DEPT data. A COSY correlation between one of the H-3 protons (δ_{H} 1.16) and a proton at δ_{H} 1.49 allowed assignment of one of the H-2 protons which in turn showed a correlation with a proton at δ_{H} 0.92 which was assigned to C-1. The remaining ¹H and ¹³C chemical shifts for C-1 and C-2 were then assigned on the basis of additional COSY and HSQC-DEPT correlations and are summarized in the table below.

¹H NMR (500 MHz, D₂O) and ¹³C NMR (125 MHz,
D₂O/TSP) Assignments of the Reb M2 aglycone.

| Position | ¹³C | ¹H |
|---|---|---|
| 1 | 41.9 | 0.92 m |
| | | 1.93 m |
| 2 | 21.8 | 1.49 m |
| | | 1.86 m |
| 3 | 39.8 | 1.16 m |
| | | 2.28 d (13.4) |
| 4 | 43.7 | — |
| 5 | 59.2 | 1.24 d (12.1) |
| 6 | 24.4 | 1.73 m |
| | | 1.94 m |
| 7 | 44.2 | 1.49 m |
| | | 1.56 m |
| 8 | 46.9 | — |
| 9 | 55.5 | 1.09 d (7.7) |
| 10 | 42.4 | — |
| 11 | 22.6 | 1.66 m |
| | | 1.70 m |
| 12 | 39.9 | 1.60 m |
| | | 2.00 m |
| 13 | 90.9 | — |
| 14 | 46.9 | 1.53 d (12.6) |
| | | 2.21 d (13.6) |
| 15 | 49.4 | 2.15 d (17.2) |
| | | 2.18 d (18.1) |
| 16 | 164.0 | — |
| 17 | 107.0 | 4.98 s |
| | | 5.16 s |
| 18 | 31.0 | 1.29 s |
| 19 | 181.5 | — |
| 20 | 19.1 | 0.92 s |

The other tertiary methyl singlet, observed at δ_{H} 0.92 showed HMBC correlations to C-1 and C-5 and was assigned as C-20. The methyl protons showed additional HMBC correlations to a quaternary carbon (δ_{C} 42.4) and a methine (δ_{C} 55.5) which were assigned as C-10 and C-9, respectively. COSY correlations between H-5 (δ_{H} 1.24) and protons at δ_{H} 1.73 and 1.94 then allowed assignment of the H-6 protons which in turn showed correlations to protons at δ_{H} 1.49 and 1.56 which were assigned to C-7. The ¹³C chemical shifts for C-6 (δ_{C} 24.4) and C-7 (δ_{C} 44.2) were then determined from the HSQC-DEPT data. COSY correlations between H-9 (δ_{H} 1.09) and protons at δ_{H} 1.66 and 1.70 allowed assignment of the H-11 protons which in turn showed COSY correlations to protons at δ_{H} 1.60 and 2.00 which were assigned as the H-12 protons. The HSQC-DEPT data was then used to assign C-11 (δ_{C} 22.6) and C-12 (δ_{C} 39.9). The olefinic protons observed at δ_{H} 4.98 and 5.16 showed HMBC correlations to C-13 (δ_{C} 90.9) and were assigned to C-17 (δ_{C} 107.0 via HSQC-DEPT). The olefinic protons H-17 showed HMBC correlations to a carbon at δ_{C} 49.4 which was assigned as C-15. An additional HMBC correlation from H-9 to a methylene carbon at δ_{C} 46.9 then allowed assignment of C-14. The ¹H chemical shifts at C-14 (δ_{H} 1.53 and 2.21) and C-15 (δ_{H} 2.15 and 2.18) were assigned using the HSQC-DEPT data.

A summary of the key HMBC and COSY correlations used to assign the aglycone region are provided below:

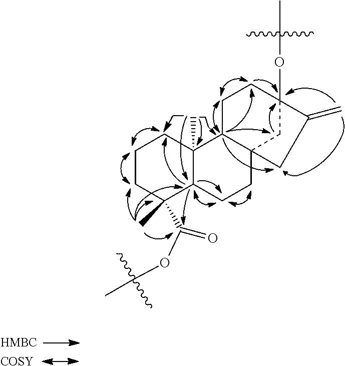

Analysis of the ¹H-¹³C HSQC-DEPT data confirmed the presence of six anomeric protons. Three of the anomeric protons were well resolved at δ_{H} 5.65 (δ_{C} 95.5), 4.92 (δ_{C} 104.9), and 4.50 (δ_{C} 105.7) in the ¹H NMR spectrum. The remaining three anomeric protons observed at δ_{H} 4.85 (6c 98.4), 4.84 (6c 105.0), and 4.83 (6c 105.3) were overlapped by the residual solvent resonance in the ¹H spectrum. The anomeric proton observed at δ_{H} 5.65 showed a HMBC correlation to C-19 which indicated that it corresponds to the anomeric proton of Gl_{CI}. Similarly, the anomeric proton observed at δ_{H} 4.85 showed a HMBC correlation to C-13 allowing it to be assigned as the anomeric proton of Gl_{CII}.

The Gl_{CI} anomeric proton (δ_{H} 5.65) showed a COSY correlation to a proton at δ_{H} 3.96 which was assigned as Gl_{CI} H-2 which in turn showed a COSY correlation to a proton at δ_{H} 3.89 (Gl_{CI} H-3) which showed a COSY correlation with a proton at δ_{H} 3.71 (Gl_{CI} H-4). Due to data overlap, the COSY spectrum did not allow assignment of the H-5 or H-6 protons. Therefore, a series of 1D-TOCSY experiments were performed using selective irradiation of the Gl_{CI} anomeric proton with several different mixing times. In addition to confirming the assignments for Gl_{CI} H-2 through H-4, the 1D-TOCSY data showed a proton at δ_{H} 3.73 assigned as Gl_{CI} H-5 and a proton at δ_{H} 4.15 assigned as one of the Gl_{CI} H-6 protons. The latter proton was also used for 1D-TOCSY experiments. The selective irradiation of H-6 with several different mixing times also confirmed the assignment of Gl_{CI} H-1 to H-5 as well as the remaining methylene proton of H-6 (δ_{H} 4.00). Assignment of the ¹³C chemical shifts for Gl_{CI} C-2 (δ_{C} 80.5), C-3 (δ_{C} 79.0), C-4 (δ_{C} 71.5), C-5 (δ_{C} 79.0), and C-6 (δ_{C} 70.9) was determined using the ¹H-¹³C HSQC-DEPT data to complete the assignment of Gl_{CI}. Furthermore, the presence of a methylene ¹³C resonance at δ_{C} 70.9 in the ¹H-¹³C HSQC-DEPT spectrum indicated a 1→6 sugar linkage of Gl_{CI} in the structure.

Two of the unassigned glucose moieties were assigned as substituents at C-2 and C-6 of Gl_{CI} on the basis of HMBC correlations. The anomeric proton observed at δ_{H} 4.83 showed an HMBC correlation to Gl_{CI} C-2 and was assigned as the anomeric proton of Gl_{CV}. The anomeric proton observed at δ_{H} 4.50 showed a HMBC correlation to Gl_{CI} C-6 and was assigned as the anomeric proton of Gl_{CVI}. The reciprocal HMBC correlations from Gl_{CI} H-2 to the anomeric carbon of Gl_{CV} and from Gl_{CI} H-6 to the anomeric carbon of Gl_{CVI} were also observed.

The anomeric proton of Gl_{CV} (δ_{H} 4.83) showed a COSY correlation with a proton at δ_{H} 3.32 which was assigned as Gl_{CV} H-2. The Gl_{CV} H-2 in turn showed a COSY correlation to a proton at δ_{H} 3.51 (Gl_{CV} H-3). This latter proton showed an additional correlation with a proton at δ_{H} 3.38 (Gl_{CV} H-4). H-4 also showed a COSY correlation to a proton at δ_{H} 3.55 (Gl_{CV} H-5) and Gl_{CV} H-5 in turn showed a COSY correlation to Gl_{CV} H-6 protons (δ_{H} 3.76 and 3.97). Assignment of the ¹³C chemical shifts for Gl_{CV} C-2 (δ_{C} 78.5), C-3 (6c 78.7), C-4 (6c 72.9), C-5 (6c 78.8), and C-6 (6c 63.6) was determined using the HSQC-DEPT data. HMBC correlations from Gl_{CV} H-3 to C-2 and C-4 and also from Gl_{CV} H-4 to C-3 and C-6 confirmed the assignments made above to complete the assignment of Gl_{CV}.

Another glucose moiety was assigned as a substituent at C-6 of Gl_{CI} on the basis of ¹H-¹³C HSQC-DEPT and HMBC correlations. The relatively downfield shift of a methylene ¹³C resonance of Gl_{CI} at δ_{C} 70.9 in the HSQC-DEPT spectrum indicated a 1->6 sugar linkage of Gl_{CI}. The anomeric proton observed at δ_{H} 4.50 showed a HMBC correlation to Gl_{CI} C-6 and was assigned as the anomeric proton of Gl_{CV}1. Similarly, methylene protons of Gl_{CI} showed HMBC correlations to the anomeric carbon of Gl_{CVI} and this confirmed the presence of a 1→6 sugar linkage between Gl_{CI} and Gl_{CVI}. The Gl_{CVI} anomeric proton showed a COSY correlation to a proton at δ_{H} 3.33 which was assigned as Gl_{CVI} H-2 which in turn showed a COSY correlation to a proton at δ_{H} 3.49 (Gl_{CVI} H-3). Due to data overlap, the COSY spectrum did not allow assignment of Gl_{CV} H-4 to H-6 based on the COSY correlations. Therefore, a series of 1D-TOCSY experiments were performed using selective irradiation of the Gl_{CVI} anomeric proton with different mixing times. In addition to confirming the assignments for Gl_{CVI} H-2 through H-3, the 1D-TOCSY data showed protons at δ_{H} 3.45 (Gl_{CVI} H-4) and δ_{H} 3.48 (Gl_{CVI} H-5) and protons at δ_{H} 3.92 and 3.94 assigned for Gl_{CVI} H-6 protons. Assignment of the ¹³C chemical shifts for Gl_{CVI} C-2 (δ_{C} 78.1), C-3 (δ_{C} 78.6), C-4 (δ_{C} 72.3), C-5 (δ_{C} 78.8), and C-6 (δ_{C} 64.1) was determined using the ¹H-¹³C HSQC-DEPT data to complete the assignment of Gl_{CVI}.

A summary of the ¹H and ¹³C chemical shifts for the glycoside at C-19 are found in the table below:

H NMR (500 MHz, D₂O) and ¹³C NMR (125 MHz,
D₂O/TSP) Assignments of the Reb M2 glycoside.

| Position | ¹³C | ¹H |
|---|---|---|
| Glc_{I}-1 | 95.5 | 5.65 d (7.6) |
| Glc_{I}-2 | 80.5 | 3.96 m |
| Glc_{I}-3 | 79.0 | 3.89 m |
| Glc_{I}-4 | 71.5 | 3.71 m |
| Glc_{I}-5 | 79.0 | 3.73 m |
| Glc_{I}-6 | 70.9 | 4.00 m |
| | | 4.15 d (11.7) |
| Glc_{V}-1 | 105.3* | 4.83* d (8.0) |
| Glc_{V}-2 | 78.5 | 3.32 m |
| Glc_{V}-3 | 78.7 | 3.51 m |
| Glc_{V}-4 | 72.9 | 3.38 m |
| Glc_{V}-5 | 78.8 | 3.55 m |
| Glc_{V}-6 | 63.6 | 3.76 m |
| | | 3.97 m |
| Glc_{VI}-1 | 105.7 | 4.50 d (7.9) |
| Glc_{VI}-2 | 78.1 | 3.33 m |
| Glc_{VI}-3 | 78.6 | 3.49 m |
| Glc_{VI}-4 | 72.3 | 3.45 m |
| Glc_{VI}-5 | 78.8 | 3.48 m |
| Glc_{VI}-6 | 64.1 | 3.92 m |
| | | 3.94 m |

*¹H and ¹³C values can be exchangeable with Glc_{IV}-1 of the following table.

A summary of the key HMBC, COSY, and 1D-TOCSY correlations used to assign the C-19 glycoside region are provided below:

¹H NMR (500 MHz, D₂O) and ¹³C NMR (125 MHz,
D₂O/TSP) Assignments of the Reb M2 glycoside.

| Position | ¹³C^{#} | ¹H |
|---|---|---|
| Glc_{II}-1 | 98.4 | 4.85 d (7.8) |
| Glc_{II}-2 | 81.7 | 3.75 m |
| Glc_{II}-3 | 88.0 | 3.98 m |
| Glc_{II}-4 | 71.3 | 3.54 m |
| Glc_{II}-5 | 80.5 | 3.96 m |
| Glc_{II}-6 | 63.6 | 3.45 m |
| | | 3.77 m |
| Glc_{III}-1 | 104.9 | 4.92 d (7.9) |
| Glc_{III}-2 | 76.3 | 3.32 m |
| Glc_{III}-3 | 78.8 | 3.51 m |
| Glc_{III}-4 | 73.3 | 3.26 t (9.5) |
| Glc_{III}-5 | 78.8 | 3.44 m |
| Glc_{III}-6 | 64.4 | 3.75 m |
| | | 3.94 m |
| Glc_{IV}-1 | 105.0 | 4.84 d (7.8) |
| Glc_{IV}-2 | 76.1 | 3.41 m |
| Glc_{IV}-3 | 78.8 | 3.46 m |
| Glc_{IV}-4 | 72.5 | 3.45 m |
| Glc_{IV}-5 | 81.7 | 3.75 m |
| Glc_{IV}-6 | 65.8 | 3.55 m |
| | | 3.78 m |

Assignment of Gl_{CII} was carried out in a similar manner. The Gl_{CII} anomeric proton (δ_{H} 4.85) showed a COSY correlation to a proton at δ_{H} 3.75 which was assigned as Gl_{CII} H-2 which in turn showed a COSY correlation to a proton at δ_{H} 3.98 (Gl_{CII} H-3). This latter proton showed an additional correlation with a proton at δ_{H} 3.54 (Gl_{CII} H-4). H-4 also showed a COSY correlation to a proton at δ_{H} 3.96 (Gl_{CII} H-5). Gl_{CII} H-5 also showed a COSY correlation to Gl_{CII} H-6 protons (δ_{H} 3.77 and 3.45). Assignment of the ¹³C chemical shifts for Gl_{CII} C-2 (δ_{C} 81.7), C-3 (δ_{C} 88.0), C-4 (δ_{C} 71.3), C-5 (δ_{C} 80.5), and C-6 (δ_{C} 63.6) was determined using the HSQC-DEPT data. HMBC correlations from Gl_{CII} H-3 to C-2 and C-4 and also from Gl_{CII} H-4 to C-3 and C-6 confirmed the assignments made above to complete the assignment of Gl_{CII}.

Two of the remaining unassigned glucose moieties were assigned as substituents at C-2 and C-3 of Gl_{CII} on the basis of HMBC correlations. The anomeric proton observed at δ_{H} 4.92 showed a HMBC correlation to Gl_{CII} C-2 and was assigned as the anomeric proton of Gl_{CII}. The anomeric proton observed at δ_{H} 4.84 showed a HMBC correlation to Gl_{CII} C-3 and was assigned as the anomeric proton of Gl_{CIV}. The reciprocal HMBC correlations between Gl_{CII} H-2 and the anomeric carbon of Gl_{CII} and between Gl_{CII} H-3 and the anomeric carbon of Gl_{CIV} were also observed.

The anomeric proton of Gl_{CII} (δ_{H} 4.92) showed a COSY correlation with a proton at δ_{H} 3.32 which was assigned as Gl_{CII} H-2. Due to data overlap, the COSY spectrum did not allow assignment of H-3 to H-6 protons. Therefore, a series of 1D-TOCSY experiments were performed using selective irradiation of the Gl_{CII} anomeric proton with different mixing times. In addition to confirming the assignments for Gl_{CII} H-2, the 1D-TOCSY data showed protons at δ_{H} 3.51 (Gl_{CII} H-3), δ_{H} 3.26 (Gl_{CII} H-4), and δ_{H} 3.44 (Gl_{CII} H-5). Once H-4 was assigned using 1D-TOCSY data, COSY correlations from H-4 to H-5 and in turn to H-6 were used to assign H-6. In the COSY spectrum, Gl_{CII} H-4 showed a correlation to Gl_{CII} H-5, which in turn showed COSY correlations to δ_{H} 3.94 and 3.75 of Gl_{CII} H-6a and H-6b, respectively. The ¹³C chemical shifts for Gl_{CII} C-2 (δ_{C} 76.3), C-3 (δ_{C} 78.8), C-4 (δ_{C} 73.3), C-5 (δ_{C} 78.8), and C-6 (δ_{C} 64.4) were then determined using the ¹H-¹³C HSQC-DEPT correlations to complete the assignment of Gl_{CII}.

The anomeric proton of Gl_{CIV} (δ_{H} 4.84) which showed a COSY correlation to a proton at δ_{H} 3.41 was assigned as Gl_{CIV} H-2 which in turn showed a COSY correlation to a proton at δ_{H} 3.46 (Gl_{CIV} H-3). This latter proton showed an additional correlation with a proton at δ_{H} 3.45 (Gl_{CIV} H-4) which also showed a COSY correlation to a proton at δ_{H} 3.75 (Gl_{CIV} H-5). Gl_{CIV} H-5 also showed a COSY correlation to Gl_{CIV} H-6 protons (δ_{H} 3.55 and 3.78). Assignment of the ¹³C chemical shifts for Gl_{CIV} C-2 (δ_{C} 76.1), C-3 (δ_{C} 78.8), C-4 (δ_{C} 72.5), C-5 (δ_{C} 81.7), and C-6 (δ_{C} 65.8) was determined using the HSQC-DEPT data. HMBC correlations from Gl_{CIV} H-3 to C-4 and C-5 and also from Gl_{CIV} H-4 to C-3 and C-6 confirmed the assignments made above to complete the assignment of Gl_{CIV}.

A summary of the ¹H and ¹³C chemical shifts for the glycoside at C-13 are found in the following table:

¹H NMR (500 MHz, D₂O) and ¹³C NMR (125 MHz,
D₂O/TSP) Assignments of the Reb M2 glycoside.

| Position | ¹³C^{#} | ¹H |
|---|---|---|
| Glc_{II}-1 | 98.4 | 4.85 d (7.8) |
| Glc_{II}-2 | 81.7 | 3.75 m |
| Glc_{II}-3 | 88.0 | 3.98 m |
| Glc_{II}-4 | 71.3 | 3.54 m |
| Glc_{II}-5 | 80.5 | 3.96 m |
| Glc_{II}-6 | 63.6 | 3.45 m |
| | | 3.77 m |
| Glc_{III}-1 | 104.9 | 4.92 d (7.9) |
| Glc_{III}-2 | 76.3 | 3.32 m |
| Glc_{III}-3 | 78.8 | 3.51 m |
| Glc_{III}-4 | 73.3 | 3.26 t (9.5) |
| Glc_{III}-5 | 78.8 | 3.44 m |
| Glc_{III}-6 | 64.4 | 3.75 m |
| | | 3.94 m |
| Glc_{IV}-1 | 105.0 | 4.84 d (7.8) |
| Glc_{IV}-2 | 76.1 | 3.41 m |
| Glc_{IV}-3 | 78.8 | 3.46 m |
| Glc_{IV}-4 | 72.5 | 3.45 m |
| Glc_{IV}-5 | 81.7 | 3.75 m |
| Glc_{IV}-6 | 65.8 | 3.55 m |
| | | 3.78 m |

A summary of the key HMBC, COSY, and 1D-TOCSY correlations used to assign the C-13 glycoside region are provided below:

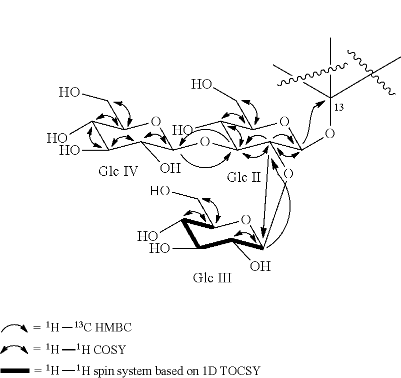

NMR and MS analyses allowed a full assignment of its structure, shown below. The chemical name of the compound is 13-[(2-O-β-D-glucopyranosyl-3-O-β-D-glucopyranosyl-β-D-glucopyranosyl)oxy] ent-kaur-16-en-19-oic acid-[(2-O-β-D-glucopyranosyl-6-O-β-D-glucopyranosyl-β-D-glucopyranosyl) ester] (rebaudioside M2 or reb M2). The compound is an isomer of rebaudioside M.

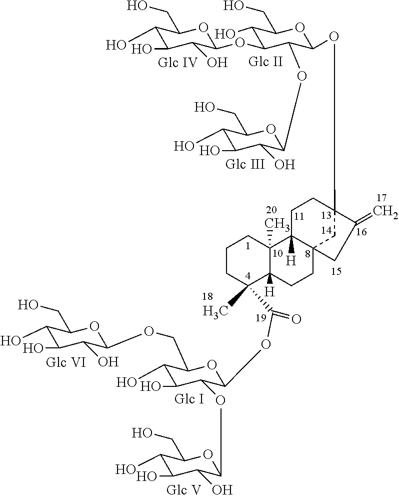

### Example 41

### Directed Evolution of UGT76G1 for the Conversion of Rebaudioside D to Rebaudioside M(Round 2)

The most active clone from the first round of directed evolution of UGT76G1 (see EXAMPLE 26 UGT76Glvar94 containing mutations: Q266E_P272A_R334K_G348P_L379G) was chosen as baseline clone for round 2. A list of 53 mutations was established containing different identified positive mutations from the first round and new mutations obtained by DNA2.0 ProteinGPStm strategy. This list of mutations was subsequently used to design 92 variant genes that contained each 3 different mutations. After codon-optimized for expression in E. coli the genes were synthesized, subcloned in the pET30a+ plasmid and used for transformation of E. coli BL21 (DE3) chemically competent cells. The obtained cells were grown in Petri-dishes on solid LB medium in the presence of Kanamycin. Suitable colonies were selected and allowed to grow in liquid LB medium in tubes. Glycerol was added to the suspension as cryoprotectant and 400 μL aliquots were stored at −20° C. and at −80° C.

These storage aliquots of E. coli BL21(DE3) containing the pET30a+_UGT76Glvar plasmids were thawed and added to LBGKP medium (20 g/L Luria Broth Lennox; 50 mM PIPES buffer pH 7.00; 50 mM Phosphate buffer pH 7.00; 2.5 g/L glucose and 50 mg/L of Kanamycine). This culture was allowed to shake in a 96 microtiter plate at 30° C. for 8 h.

3.95 mL of production medium containing 60 g/L of Overnight Express™ Instant TB medium (Novagen®), 10 g/L of glycerol and 50 mg/L of Kanamycin was inoculated with 50 μL of above described culture. In a 48 deepwell plate the resulting culture was allowed to stir at 20° C. The cultures gave significant growth and a good OD (600 nm) was obtained. After 44 h, the cells were harvested by centrifugation and frozen.

Lysis was performed by addition of Bugbuster® Master mix (Novagen®) to the thawed cells and the lysate was recovered by centrifugation. Activity tests were performed with 100 μL of fresh lysate that was added to a solution of Rebaudioside D (final concentration 0.5 mM), MgCl₂ (final concentration 3 mM) and UDP-Glucose (final concentration 2.5 mM) in 50 mM phosphate buffer pH 7.2.

The reaction was allowed to run at 30° C. and samples were taken after 2, 4, 7 and 24 h. to determine conversion and initial rate by HPLC (CAD detection) using the analytical method that was described above for the transformation of Rebaudioside D to Rebaudioside M. In parallel the experiments were performed with baseline clone, Round1-Var94. The conversion after 22 h. and initial rate for this baseline clone was defined as 10000 and the normalized conversions and initial rates for the round 2 clones are depicted in the following table:

| | | Normalized conversion | Normalized initial |
|---|---|---|---|
| Clone | Mutations* | Reb D to Reb Mafter 22 h. | rate (0-4 h) |
| Round1-Var94 | UGT76G1 | 100% | 100% |
| | (Q266E_P272A_R334K_G348P_L379G) | | |
| | baseline clone | | |
| Round2-Var1 | Round1-Var94 (A213N_P348G_I411V) | 70% | 86% |
| Round2-Var2 | Round1-Var94 (K303G_I423M_Q425E) | 120% | 134% |
| Round2-Var3 | Round1-Var94 (V20L_N138K_S147G) | 14% | 15% |
| Round2-Var4 | Round1-Var94 (I16V_V133A_L299I) | 37% | 43% |
| Round2-Var5 | Round1-Var94 (S241V_S274G_Q432E) | 75% | 72% |
| Round2-Var6 | Round1-Var94 (I16V_L139V_I218V) | 62% | 68% |
| Round2-Var7 | Round1-Var94 (K334R_N409K_Q432E) | 104% | 92% |
| Round2-Var8 | Round1-Var94 (I15L_R141T_I407V) | 17% | 26% |
| Round2-Var9 | Round1-Var94 (R141T_K303G_G379L) | 31% | 42% |
| Round2-Var10 | Round1-Var94 (I190L_K303G_P348G) | 131% | 149% |
| Round2-Var11 | Round1-Var94 (E266Q_F314S_N409R) | 106% | 132% |
| Round2-Var12 | Round1-Var94 (V133A_I295V_K303G) | 43% | 49% |
| Round2-Var13 | Round1-Var94 (I16V_S241V_N409R) | 80% | 79% |
| Round2-Var14 | Round1-Var94 (A239V_K334R_G379L) | 58% | 55% |
| Round2-Var15 | Round1-Var94 (I190L_K393R_V396L) | 118% | 126% |
| Round2-Var16 | Round1-Var94 (L101F_I295M_K393R) | 84% | 89% |
| Round2-Var17 | Round1-Var94 (A239V_E266Q_Q425E) | 96% | 101% |
| Round2-Var18 | Round1-Var94 (V20L_I190L_I423M) | 98% | 98% |
| Round2-Var19 | Round1-Var94 (V20L_G379L_S456L) | 84% | 81% |
| Round2-Var20 | Round1-Var94 (K334R_P348G_N409R) | 73% | 73% |
| Round2-Var21 | Round1-Var94 (E231A_S241V_E449D) | 53% | 50% |
| Round2-Var22 | Round1-Var94 (K188R_L299I_V394I) | 56% | 59% |
| Round2-Var23 | Round1-Var94 (E231A_S274G_V394I) | 110% | 124% |
| Round2-Var24 | Round1-Var94 (S42A_I295V_Q432E) | 71% | 78% |
| Round2-Var25 | Round1-Var94 (A213N_A272P_K334R) | 95% | 80% |
| Round2-Var26 | Round1-Var94 (L158Y_S274K_N409K) | 80% | 50% |
| Round2-Var27 | Round1-Var94 (K188R_I295M_Q425E) | 132% | 116% |
| Round2-Var28 | Round1-Var94 (I15L_I295M_V394I) | 53% | 36% |
| Round2-Var29 | Round1-Var94 (V133A_A239V_V394I) | 47% | 30% |
| Round2-Var30 | Round1-Var94 (L158Y_F314S_K316R) | 107% | 72% |
| Round2-Var31 | Round1-Var94 (L158Y_A239V_A272P) | 54% | 30% |
| Round2-Var32 | Round1-Var94 (F46I_D301N_V396L) | 109% | 101% |
| Round2-Var33 | Round1-Var94 (L101F_I218V_Q432E) | 78% | 54% |
| Round2-Var34 | Round1-Var94 (I16V_F46I_I295M) | 110% | 95% |
| Round2-Var35 | Round1-Var94 (A213N_E266S_I407V) | 98% | 79% |
| Round2-Var36 | Round1-Var94 (A239V_S274K_I295M) | 102% | 89% |
| Round2-Var37 | Round1-Var94 (A239V_F314S_S450K) | 105% | 99% |
| Round2-Var38 | Round1-Var94 (L139V_K188R_D301N) | 66% | 51% |
| Round2-Var39 | Round1-Var94 (I45V_I218V_S274K) | 87% | 58% |
| Round2-Var40 | Round1-Var94 (S241V_K303G_V394I) | 78% | 57% |
| Round2-Var41 | Round1-Var94 (R141T_S274G_K334R) | 41% | 28% |
| Round2-Var42 | Round1-Var94 (V217L_S274G_L299I) | 47% | 34% |
| Round2-Var43 | Round1-Var94 (S274G_D301N_P348G) | 98% | 91% |
| Round2-Var44 | Round1-Var94 (E231A_N409R_S450K) | 87% | 65% |
| Round2-Var45 | Round1-Var94 (R64H_E231A_K316R) | 88% | 64% |
| Round2-Var46 | Round1-Var94 (V394I_N409K_I411V) | 110% | 100% |
| Round2-Var47 | Round1-Var94 (I45V_I295M_K303G) | 113% | 88% |
| Round2-Var48 | Round1-Var94 (L101F_V396L_L398V) | 46% | 43% |
| Round2-Var49 | Round1-Var94 (N27S_L101F_S447A) | 54% | 37% |
| Round2-Var50 | Round1-Var94 (S274G_F314S_L398V) | 129% | 156% |
| Round2-Var51 | Round1-Var94 (E266Q_L299I_K393R) | 70% | 51% |
| Round2-Var52 | Round1-Var94 (V217L_E266S_V394I) | 62% | 48% |
| Round2-Var53 | Round1-Var94 (N138K_A272P_N409R) | 118% | 102% |
| Round2-Var54 | Round1-Var94 (E266S_F314S_Q432E) | 124% | 146% |
| Round2-Var55 | Round1-Var94 (D301N_G379L_L398V) | 56% | 45% |
| Round2-Var56 | Round1-Var94 (F46I_E266S_K334R) | 123% | 142% |
| Round2-Var57 | Round1-Var94 (A272P_V394I_Q432E) | 133% | 142% |
| Round2-Var58 | Round1-Var94 (V394I_I407V_S456L) | 118% | 114% |
| Round2-Var59 | Round1-Var94 (I218V_E266Q_I423M) | 106% | 98% |
| Round2-Var60 | Round1-Var94 (A272P_G379L_I407V) | 80% | 63% |
| Round2-Var61 | Round1-Var94 (E231A_K303G_S456L) | 113% | 110% |
| Round2-Var62 | Round1-Var94 (I190L_E266Q_I407V) | 150% | 167% |
| Round2-Var63 | Round1-Var94 (N27S_L139V_I295V) | 43% | 25% |
| Round2-Var64 | Round1-Var94 (V217L_I423M_S447A) | 67% | 51% |
| Round2-Var65 | Round1-Var94 (L158Y_E266S_E449D) | 68% | 43% |
| Round2-Var66 | Round1-Var94 (S42A_F46I_I407V) | 160% | 203% |
| Round2-Var67 | Round1-Var94 (N138K_E231A_D301N) | 118% | 93% |
| Round2-Var68 | Round1-Var94 (K188R_G379L_N409R) | 52% | 35% |
| Round2-Var69 | Round1-Var94 (I15L_E231A_V396L) | 38% | 22% |
| Round2-Var70 | Round1-Var94 (E231A_Q425E_Q432E) | 115% | 119% |
| Round2-Var71 | Round1-Var94 (D301N_K316R_Q425E) | 126% | 121% |
| Round2-Var72 | Round1-Var94 (L139V_I295M_F314S) | 76% | 91% |
| Round2-Var73 | Round1-Var94 (S147G_E266S_D301N) | 30% | 18% |
| Round2-Var74 | Round1-Var94 (R64H_S147G_S447A) | 23% | 12% |
| Round2-Var75 | Round1-Var94 (S42A_K303G_L398V) | 95% | 110% |
| Round2-Var76 | Round1-Var94 (I45V_D301N_E449D) | 62% | 60% |
| Round2-Var77 | Round1-Var94 (V133A_E266S_I411V) | 37% | 28% |
| Round2-Var78 | Round1-Var94 (I45V_N409R_Q425E) | 63% | 59% |
| Round2-Var79 | Round1-Var94 (R141T_A272P_F314S) | 23% | 10% |
| Round2-Var80 | Round1-Var94 (E266S_S274G_N409R) | 81% | 91% |
| Round2-Var81 | Round1-Var94 (N409K_Q425E_S450K) | 81% | 84% |
| Round2-Var82 | Round1-Var94 (N27S_R64H_K393R) | 47% | 37% |
| Round2-Var83 | Round1-Var94 (S42A_A213N_V217L) | 62% | 46% |
| Round2-Var84 | Round1-Var94 (N27S_S274K_I407V) | 49% | 44% |
| Round2-Var85 | Round1-Var94 (I411V_Q425E_S456L) | 75% | 81% |
| Round2-Var86 | Round1-Var94 (A239V_K316R_E449D) | 83% | 72% |
| Round2-Var87 | Round1-Var94 (S147G_A239V_P348G) | 18% | 7% |
| Round2-Var88 | Round1-Var94 (V20L_S274G_S450K) | 71% | 68% |
| Round2-Var89 | Round1-Var94 (F314S_V394I_S447A) | 88% | 123% |
| Round2-Var90 | Round1-Var94 (R64H_E266Q_I295M) | 45% | 47% |
| Round2-Var91 | Round1-Var94 (N138K_I295V_I407V) | 50% | 51% |
| Round2-Var92 | Round1-Var94 (I15L_P348G_Q432E) | 18% | 13% |

*Mutations are noted as follows: reference gene-original amino acid-position-new amino acid: For example the mutation of an alanine at position 33 to a glycine for variant 94 from the first round of directed evolution of UGT76G1 is noted as Round1-Var94 (A33G)

Modeling of these results allowed to obtain a ranking of the effect of each mutation. The following mutations were determined as being beneficial for activity: S42A, F461, 1190L, S274G, 1295M, K303G, F314S, K1316R, K1393R, V3941, 1407V, N409K, N409R, Q425E, Q432E, S447A, S456L.

### Example 42

### In Vivo Production of AtSUS

AtSUS
>gi|79328294|ref|NP_001031915.1| sucrose
synthase 1 [Arabidopsis thaliana]
SEQ ID 13
MANAERMITRVHSQRERLNETLVSERNEVLALLSRVEAKGKGILQQNQII
AEFEALPEQTRKKLEGGPFFDLLKSTQEAIVLPPWVALAVRPRPGVWEYL
RVNLHALVVEELQPAEFLHFKEELVDGVKNGNFTLELDFEPFNASIPRPT
LHKYIGNGVDFLNRHLSAKLFHDKESLLPLLKFLRLHSHQGKNLMLSEKI
QNLNTLQHTLRKAEEYLAELKSETLYEEFEAKFEEIGLERGWGDNAERVL
DMIRLLLDLLEAPDPCTLETFLGRVPMVFNVVILSPHGYFAQDNVLGYPD
TGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVEL
SKELNGKPDLIIGNYSDGNLVASLLAHKLGVTQCTIAHALEKTKYPDSDI
YWKKLDDKYHFSCQFTADIFAMNHTDFIITSTFQEIAGSKETVGQYESHT
AFTLPGLYRVVHGIDVFDPKFNIVSPGADMSIYFPYTEEKRRLTKFHSEI
EELLYSDVENKEHLCVLKDKKKPILFTMARLDRVKNLSGLVEWYGKNTRL
RELANLVVVGGDRRKESKDNEEKAEMKKMYDLIEEYKLNGQFRWISSQMD
RVRNGELYRYICDTKGAFVQPALYEAFGLTVVEAMTCGLPTFATCKGGPA
EIIVHGKSGFHIDPYHGDQAADTLADFFTKCKEDPSHWDEISKGGLQRIE
EKYTWQIYSQRLLTLTGVYGFWKHVSNLDRLEARRYLEMFYALKYRPLAQ
AVPLAQDD

The synthetic gene of AtSuS that was codon optimized for expression in E. coli and subcloned in the pET30a+ plasmid using the NdeI and XhoI restriction sites. The pET30A+ vector containing the AtSUS gene was used to transform electrocompetent E. coli B121(DE3) cells. The obtained cells were grown in petri-dishes in the presence of Kanamycin and suitable colonies were selected and allowed to grow in liquid LB medium (erlenmeyer flasks). Glycerol was added to the suspension as cryoprotectant and 400 μL aliquots were stored at −20° C. and at −80° C.

The storage aliquots of E. coli BL21(DE3) containing the pET30A+_AtSUS plasmids were thawed and added to 30 mL of LBGKP medium (20 g/L Luria Broth Lennox; 50 mM PIPES buffer pH 7.00; 50 mM Phosphate buffer pH 7.00; 2.5 g/L glucose and 50 mg/L of Kanamycine). This culture was allowed to shake at 135 rpm at 30° C. for 8 h.

The production medium contained 60 g/L of overnight express instant TB medium (Novagen), 10 g/L of glycerol and 50 mg/L of Kanamycine. The preculture was added to 800 mL of this medium and the solution was allowed to stir at 20° C. while taking samples to measure the OD and pH. The culture gave significant growth and a good OD was obtained. After 40 h, the cells were harvested by centrifugation and frozen to obtain 30.1 g of cell wet weight.

Lysis was performed by Fastprep (MP Biomedicals, Lysing matrix B, speed 6.0, 3×40 sec) with a cell suspension of 200 mg of cells in 1.0 mL of 50 mM Tris buffer pH 7.5. The lysate was recovered by centrifugation and used fresh.

### Example 43

Conversion of Rebaudioside a to Rebaudioside M with In Situ Prepared UDP-Glucose Using UGTSL2, UGT76G1-R1-F12 and AtSUS

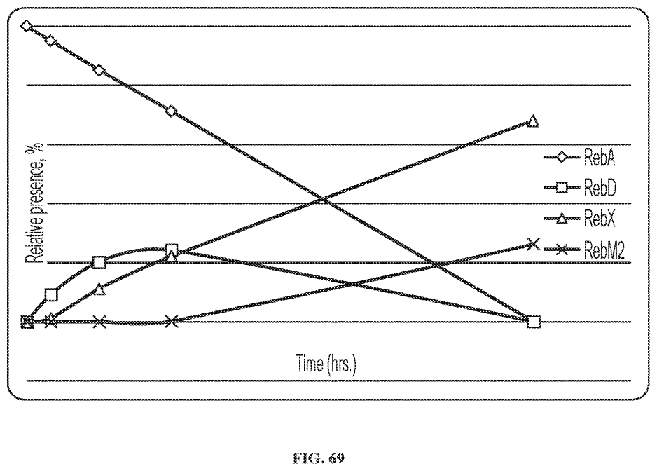

The reaction was performed at 1 mL scale using 100 mM of sucrose, 3 mM of MgCl₂, 0.25 mM of UDP and 0.5 mM of Rebaudioside A in potassium phosphate buffer (50 mM final concentration, pH 7.5). The reaction was started by adding 15 μL of UGTSL2 (see EXAMPLE 27) lysate (2 U/mL), 150 μL of UGT76Glvar94 (see EXAMPLE 26) (2.5 U/mL) and 15 μL of AtSUS (see EXAMPLE 42) (400 U/mL). The reaction was followed by HPLC after quenching 125 μL samples with 10 μL of 2 N H₂SO₄ and 115 μL of 60% methanol. 68% of Rebaudioside M and 26% of Rebaudioside M2 was obtained after 21 h of reaction time. The results are presented in FIG. 69.

Although various embodiments of the present invention have been disclosed in the foregoing description for purposes of illustration, it should be understood that a variety of changes, modifications and substitutions may be incorporated without departing from either the spirit of scope of the present invention.

1.-15. (canceled)

16. A consumable product comprising a target steviol glycoside selected from the group consisting of stevioside, reb A, reb E, reb D, reb D2, reb M, and reb M2, wherein the product is selected from the group consisting of a food, a beverage, a pharmaceutical composition, a tobacco product, a nutraceutical composition, an oral hygiene composition, and a cosmetic composition.

17. The consumable product of claim 16, wherein the target steviol glycoside is reb D.

18. The consumable product claim 16, wherein the target steviol glycoside is reb M.

19. (canceled)

20. (canceled)

21. The consumable product of claim 16, wherein the target steviol glycoside is reb D2.

22. The consumable product of claim 16, wherein the composition is selected from the group consisting of beverages; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco and medicinal products.

23.-27. (canceled)

28. The consumable product of claim 16, wherein the target steviol glycoside is reb M2.

29.-32. (canceled)

33. The consumable product of claim 16, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

34. The consumable product of claim 16, further comprising at least one functional ingredient selected from the group consisting of saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

35. The consumable product of claim 16, further comprising a compound selected from the group consisting of reb A, reb B, reb D, NSF-02, Mogroside V, Luo Han Guo, allulose, allose, D-tagatose, erythritol and combinations thereof.

36.-43. (canceled)


SEQUENCE LISTING (13 sequences)

SEQ ID NO: 1 - DNA, 1397 bp, Escherichia coli
  source 1..1397: mol_type = genomic DNA
ccatggcccatatggaaaacaaaaccgaaaccaccgttcgtcgtcgtcgccgtattattctgtttccggttccgtttcagggtcatattaatccgattctgcagctggcaaatgtgctgtatagcaaaggttttagcattaccatttttcataccaattttaacaaaccgaaaaccagcaattatccgcattttacctttcgctttattctggataatgatccgcaggatgaacgcattagcaatctgccgacacatggtccgctggcaggtatgcgtattccgattattaacgaacatggtgcagatgaactgcgtcgtgaactggaactgctgatgctggcaagcgaagaagatgaagaagttagctgtctgattaccgatgcactgtggtattttgcacagagcgttgcagatagcctgaatctgcgtcgtctggttctgatgaccagcagcctgtttaactttcatgcacatgttagcctgccgcagtttgatgaactgggttatctggatccggatgataaaacccgtctggaagaacaggcaagcggttttccgatgctgaaagtgaaagatatcaaaagcgcctatagcaattggcagattctgaaagaaattctgggcaaaatgattaaacagaccaaagcaagcagcggtgttatttggaatagctttaaagaactggaagaaagcgaactggaaaccgtgattcgtgaaattccggcaccgagctttctgattccgctgccgaaacatctgaccgcaagcagcagcagcctgctggatcatgatcgtaccgtttttcagtggctggatcagcagcctccgagcagcgttctgtatgttagctttggtagcaccagcgaagttgatgaaaaagattttctggaaattgcccgtggtctggttgatagcaaacagagctttctgtgggttgttcgtccgggttttgttaaaggtagcacctgggttgaaccgctgccggatggttttctgggtgaacgtggtcgtattgttaaatgggttccgcagcaagaagttctggcacacggcgcaattggtgcattttggacccatagcggttggaatagcaccctggaaagcgtttgtgaaggtgttccgatgatttttagcgattttggtctggatcagccgctgaatgcacgttatatgagtgatgttctgaaagtgggtgtgtatctggaaaatggttgggaacgtggtgaaattgcaaatgcaattcgtcgtgttatggtggatgaagaaggtgaatatattcgtcagaatgcccgtgttctgaaacagaaagcagatgttagcctgatgaaaggtggtagcagctatgaaagcctggaaagtctggttagctatattagcagcctgtaataactcgag

SEQ ID NO: 2 - DNA, 1442 bp, Escherichia coli
  source 1..1442: mol_type = genomic DNA
ccatggcacatatggcaaccagcgatagcattgttgatgatcgtaaacagctgcatgttgcaacctttccgtggctggcatttggtcatattctgccgtatctgcagctgagcaaactgattgcagaaaaaggtcataaagtgagctttctgagcaccacccgtaatattcagcgtctgagcagccatattagtccgctgattaatgttgttcagctgaccctgcctcgtgttcaagaactgccggaagatgccgaagcaaccaccgatgttcatccggaagatattccgtatctgaaaaaagcaagtgatggtctgcagccggaagttacccgttttctggaacagcatagtccggattggatcatctatgattatacccattattggctgccgagcattgcagcaagcctgggtattagccgtgcacattttagcgttaccaccccgtgggcaattgcatatatgggtccgagcgcagatgcaatgattaatggtagtgatggtcgtaccaccgttgaagatctgaccacccctccgaaatggtttccgtttccgaccaaagtttgttggcgtaaacatgatctggcacgtctggttccgtataaagcaccgggtattagtgatggttatcgtatgggtctggttctgaaaggtagcgattgtctgctgagcaaatgctatcatgaatttggcacccagtggctgccgctgctggaaaccctgcatcaggttccggttgttccggtgggtctgctgcctccggaagttccgggtgatgaaaaagatgaaacctgggttagcatcaaaaaatggctggatggtaaacagaaaggtagcgtggtttatgttgcactgggtagcgaagttctggttagccagaccgaagttgttgaactggcactgggtctggaactgagcggtctgccgtttgtttgggcatatcgtaaaccgaaaggtccggcaaaaagcgatagcgttgaactgccggatggttttgttgaacgtacccgtgatcgtggtctggtttggaccagctgggcacctcagctgcgtattctgagccatgaaagcgtttgtggttttctgacccattgtggtagcggtagcattgtggaaggtctgatgtttggtcatccgctgattatgctgccgatttttggtgatcagccgctgaatgcacgtctgctggaagataaacaggttggtattgaaattccgcgtaatgaagaagatggttgcctgaccaaagaaagcgttgcacgtagcctgcgtagcgttgttgttgaaaaagaaggcgaaatctataaagccaatgcacgtgaactgagcaaaatctataatgataccaaagtggaaaaagaatatgtgagccagttcgtggattatctggaaaaaaacacccgtgcagttgccattgatcacgaaagctaatgactcgag

SEQ ID NO: 3 - protein, 472 aa, Oryza sativa
MDDAHSSQSPLHVVIFPWLAFGHLLPCLDLAERLAARGHRVSFVSTPRNLARLPPVRPELAELVDLVALPLPRVDGLPDGAEATSDVPFDKFELHRKAFDGLAAPFSAFLDTACAGGKRPDWVLADLMHHWVALASQERGVPCAMILPCSAAVVASSAPPTESSADQREAIVRSMGTAAPSFEAKRATEEFATEGASGVSIMTRYSLTLQRSKLVAMRSCPELEPGAFTILTRFYGKPVVPFGLLPPRPDGARGVSKNGKHDAIMQWLDAQPAKSVVYVALGSEAPMSADLLRELAHGLDLAGTRFLWAMRKPAGVDADSVLPAGFLGRTGERGLVTTRWAPQVSILAHAAVCAFLTHCGWGSVVEGLQFGHPLIMLPILGDQGPNARILEGRKLGVAVPRNDEDGSFDRGGVAGAVRAVVVEEEGKTFFANARKLQEIVADREREERCIDEFVQHLTSWNELKNNSDGQYP

SEQ ID NO: 4 - protein, 502 aa, Avena strigosa
MAVKDEQQSPLHILLFPFLAPGHLIPIADMAALFASRGVRCTILTTPVNAAIIRSAVDRANDAFRGSDCPAIDISVVPFPDVGLPPGVENGNALTSPADRLKFFQAVAELREPFDRFLADNHPDAVVSDSFFHWSTDAAAEHGVPRLGFLGSSMFAGSCNESTLHNNPLETAADDPDALVSLPGLPHRVELRRSQMMDPKKRPDHWALLESVNAADQKSFGEVFNSFHELEPDYVEHYQTTLGRRTWLVGPVALASKDMAGRGSTSARSPDADSCLRWLDTKQPGSVVYVSFGTLIRFSPAELHELARGLDLSGKNFVWVLGRAGPDSSEWMPQGFADLITPRGDRGFIIRGWAPQMLILNHRALGGFVTHCGWNSTLESVSAGVPMVTWPRFADQFQNEKLIVEVLKVGVSIGAKDYGSGIENHDVIRGEVIAESIGKLMGSSEESDAIQRKAKDLGAEARSAVENGGSSYNDVGRLMDELMARRSSVKVGEDIIPTNDGL

SEQ ID NO: 5 - protein, 470 aa, Solanum lycopersicum
MSPKLHKELFFHSLYKKTRSNHTMATLKVLMFPFLAYGHISPYLNVAKKLADRGFLIYFCSTPINLKSTIEKIPEKYADSIHLIELHLPELPQLPPHYHTTNGLPPNLNQVLQKALKMSKPNFSKILQNLKPDLVIYDILQRWAKHVANEQNIPAVKLLTSGAAVFSYFFNVLKKPGVEFPFPGIYLRKIEQVRLSEMMSKSDKEKELEDDDDDDDLLVDGNMQIMLMSTSRTIEAKYIDFCTALTNWKVVPVGPPVQDLITNDVDDMELIDWLGTKDENSTVFVSFGSEYFLSKEDMEEVAFALELSNVNFIWVARFPKGEERNLEDALPKGFLERIGERGRVLDKFAPQPRILNHPSTGGFISHCGWNSAMESIDFGVPIIAMPMHLDQPMNARLIVELGVAVEIVRDDDGKIHRGEIAETLKGVITGKTGEKLRAKVRDISKNLKTIRDEEMDAAAEELIQLCRNGN

SEQ ID NO: 6 - protein, 464 aa, Oryza sativa
MHVVMLPWLAFGHILPFAEFAKRVARQGHRVTLFSTPRNTRRLIDVPPSLAGRIRVVDIPLPRVEHLPEHAEATIDLPSNDLRPYLRRAYDEAFSRELSRLLQETGPSRPDWVLADYAAYWAPAAASRHGVPCAFLSLFGAAALCFFGPAETLQGRGPYAKTEPAHLTAVPEYVPFPTTVAFRGNEARELFKPSLIPDESGVSESYRFSQSIEGCQLVAVRSNQEFEPEWLELLGELYQKPVIPIGMFPPPPPQDVAGHEETLRWLDRQEPNSVVYAAFGSEVKLTAEQLQRIALGLEASELPFIWAFRAPPDAGDGDGLPGGFKERVNGRGVVCRGWVPQVKFLAHASVGGFLTHAGWNSIAEGLANGVRLVLLPLMFEQGLNARQLAEKKVAVEVARDEDDGSFAANDIVDALRRVMVGEEGDEFGVKVKELAKVFGDDEVNDRYVRDFLKCLSEYKMQRQG

SEQ ID NO: 7 - protein, 515 aa, Arabidopsis lyrata
MDDKKEEVMHIAMFPWLAMGHLLPFLRLSKLLAQKGHKISFISTPRNILRLPKLPSNLSSSITFVSFPLPSISGLPPSSESSMDVPYNKQQSLKAAFDLLQPPLTEFLRLSSPDWIIYDYASHWLPSIAKELGISKAFFSLFNAATLCFMGPSSSLIEESRSTPEDFTVVPPWVPFKSTIVFRYHEVSRYVEKTDEDVTGVSDSVRFGYTIDGSDAVFVRSCPEFEPEWFSLLQDLYRKPVFPIGFLPPVIEDDDDDTTWVRIKEWLDKQRVNSVVYVSLGTEASLRREELTELALGLEKSETPFFWVLRNEPQIPDGFEERVKGRGMVHVGWVPQVKILSHESVGGFLTHCGWNSVVEGIGFGKVPIFLPVLNEQGLNTRLLQGKGLGVEVLRDERDGSFGSDSVADSVRLVMIDDAGEEIREKVKLMKGLFGNMDENIRYVDELVGFMRNDESSQLKEEEEEDDCSDDQSSEVSSETDEKELNLDLKEEKRRISVYKSLSSEFDDYVANEKMG

SEQ ID NO: 8 - protein, 772 aa, Oryza sativa
MHVVICPLLAFGHLLPCLDLAQRLACGHRVSFVSTPRNISRLPPVRPSLAPLVSFVALPLPRVEGLPNGAESTHNVPHDRPDMVELHLRAFDGLAAPFSEFLGTACADWVMPTSSAPRQTLSSNIHRNSSRPGTPAPSGRLLCPITPHSNTLERAAEKLVRSSRQNARARSLLAFTSPPLPYRDVFRSLLGLQMGRKQLNIAHETNGRRTGTLPLNLCRWMWKQRRCGKLRPSDVEFNTSRSNEAISPIGASLVNLQSIQSPNPRAVLPIASSGVRAVFIGRARTSTPTPPHAKPARSAAPRAHRPPSSVMDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 9 - protein, 442 aa, Escherichia coli
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQNIPAGKLLTSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKHENSTVFVSFGSEYFLSKEDMEEVAFALELSNVNFIWVARFPKGEERNLEDALPKGFLERIGERGRVLDKFAPQPRILNHPSTGGFISHCGWNSAMESIDFGVPIIAMPIHNDQPINAKLMVELGVAVEIVRDDDGKIHRGEIAETLKSVVTGETGEILRAKVREISKNLKSIRDEEMDAVAEELIQLCRNSNKSK

SEQ ID NO: 10 - protein, 454 aa, Escherichia coli
MGTEVTVHKNTLRVLMFPWLAYGHISPFLNVAKKLVDRGFLIYLCSTAINLKSTIKKIPEKYSDSIQLIELHLPELPELPPHYHTTNGLPPHLNHTLQKALKMSKPNFSKILQNLKPDLVIYDLLQQWAEGVANEQNIPAVKLLTSGAAVLSYFFNLVKKPGVEFPFPAIYLRKNELEKMSELLAQSAKDKEPDGVDPFADGNMQVMLMSTSRIIEAKYIDYFSGLSNWKVVPVGPPVQDPIADDADEMELIDWLGKKDENSTVFVSFGSEYFLSKEDREEIAFGLELSNVNFIWVARFPKGEEQNLEDALPKGFLERIGDRGRVLDKFAPQPRILNHPSTGGFISHCGWNSVMESVDFGVPIIAMPIHLDQPMNARLIVELGVAVEIVRDDYGKIHREEIAEILKDVIAGKSGENLKAKMRDISKNLKSIRDEEMDTAAEELIQLCKNSPKLK

SEQ ID NO: 11 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 12 - protein, 470 aa, Solanum lycopersicum; identical to SEQ ID NO: 5

SEQ ID NO: 13 - protein, 808 aa, Escherichia coli
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD